(19) United States
(12) Patent Application Publication
(10) Pub. No.: US 2012/0083471 A1
(43) Pub. Date: Apr. 5, 2012

(54) Novel Compounds, Pharmaceutical Compositions Containing Same, Methods of Use for Same, and Methods for Preparing Same

(76) Inventors: Craig A. Townsend, Baltimore, MD (US); Edward Wydysh, Somerville, MA (US); Francis Kuhajda, Baltimore, MD (US); Gabriele Valeria Ronnett, Lutherville, MD (US)

(21) Appl. No.: 13/273,740

(22) Filed: Oct. 14, 2011

Related U.S. Application Data
(63) Continuation of application No. 13/002,967.
(60) Provisional application No. 61/129,578, filed on Jul. 7, 2008.

(51) Int. Cl. A61K 31/662; C07D 333/24; C07D 213/55; C07C 311/06; A61K 31/4418; A61K 31/505; A61K 31/381; A61K 31/196; C07C 311/08; C07F 9/38; C07C 311/21; C07C 311/13; A61K 31/195; A61P 3/04; A61P 3/00; C07D 239/26

(52) U.S. Cl. 514/117; 544/335; 549/77; 546/335; 562/430; 514/357; 514/256; 514/438; 514/562; 562/11

(57) ABSTRACT

The present invention relates to novel pharmaceutical compositions containing the same, and methods of use for a variety of therapeutically valuable uses including, but not limited to, treating obesity by inhibiting the activity of Glycerol 3-phosphate acyltransferase (GPAT).

### PRIORITY FILING

This application claims priority from U.S. Provisional Application No. 61/129,578, which was filed on Jul. 7, 2008 and is incorporated herein by reference.

### FIELD OF THE INVENTION

The present invention relates to novel compounds, pharmaceutical compositions containing the same, and methods of use for a variety of therapeutically valuable uses including, but not limited to, treating obesity by inhibiting the activity of Glycerol 3-phosphate acyltransferase (GPAT).

### BACKGROUND OF THE INVENTION

The incidence of obesity and other diseases associated with an increased triacylglycerol mass is increasingly recognized as a significant public health issue. Obesity is currently estimated by the World Health Organization to affect at least 400 million adults worldwide. In the U.S. alone, there are estimates that approximately two-thirds of adults are overweight or obese. Various diseases are associated with obesity, including type-2 diabetes, hypertension, cardiovascular diseases, nonalcoholic fatty liver disease, and certain types of cancer.

Even though there is a clear need for effective and widely available anti-obesity therapeutics, only two such drugs approved for long-term use in the U.S.: Orlistat functions by blocking the absorption of dietary fat, and sibutramine affects the central nervous system, reducing energy intake and increasing energy use. Although not completely ineffective, each of these drugs displays limited efficacy and produces undesirable side effects.

Anti-obesity drugs currently in development utilize a wide variety of mechanisms, involving both central and peripheral targets. Alteration of lipid metabolism, by decreasing the de novo synthesis of triglycerides while increasing oxidation of stored fats, is a peripheral mechanism. This approach, based on weight loss effects observed with the compounds C75, cerulenin, and hGH₍₁₇₇₋₁₉₁₎, may be highly valuable in developing anti-obesity drugs.

Glycerol 3-phosphate acyltransferase (GPAT) catalyzes the rate-limiting step of glycerolipid biosynthesis, the acylation of glycerol 3-phosphate with saturated long chain acyl-CoAs. At present, there are four identified GPAT family members: GPAT1, a mitochondrial isoform catalyzing the bulk of hepatic triglyceride synthesis; GPAT2, a second mitochondrial isoform that synthesizes triglycerides but is less responsive to dietary control; GPAT3, localized to the endoplasmic reticulum, is responsible for the bulk of triglyceride synthesis in adipocytes, small intestine, kidney, and heart; and GPAT4, a microsomal isoform whose function is not completely elucidated. The mitochondrial isoform of glycerol-3-phosphate acyltransferase-1 (mtGPAT) catalyzes the esterification of long chain acyl-CoAs with sn-glycerol-3-phosphate to produce lysophosphatidic acid (LPA). This reaction is thought to constitute the first committed and rate-limiting step of glycerolipid biosynthesis. The purported mechanism of this reaction is similar to that of a serine protease, with the primary hydroxyl group of glycerol-3-phosphate taking the place of serine in the catalytic triad. Next, LPA is esterified further to produce phosphatidic acid, a precursor of various phospholipids including triacylglycerol (TAG), the main component of animal fat. In addition to obesity, high TAG levels in the bloodstream have been linked to several diseases, notably atherosclerosis and pancreatitis.

It has been shown that mtGPAT1 displays a strong preference for incorporating palmitoyl-CoA (16:0), thereby primarily producing saturated phospholipids, whereas the other two enzymes are not selective. Of the three isoforms of GPAT, only mtGPAT1 is affected by changes in diet or exercise. When excess calories are available from a high-carbohydrate diet, mtGPAT1 mRNA expression increases, resulting in greater mtGPAT1 activity. It has been shown that mice that remain stationary for ten hours following a prolonged exercise regimen experience an increase in mtGPAT1 activity compared to mice that did not exercise at all, resulting in a significant overshoot of triacylglycerol (TAG) synthesis. MtGPAT1-deficient mice exhibit lower hepatic TAG levels and secrete less very low density lipoprotein (VLDL) than control mice. In contrast, rat hepatocytes with 2.7-fold increased mtGPAT1 activity demonstrated a significant increase in de novo synthesis of diacylglycerol. Overexpression of mtGPAT1 in vivo, as expected from the previous result, causes the levels of accumulated TAG and diacylglycerol (DAG) in mouse liver to rise dramatically to 12-fold and 7-fold that of normal levels. In addition to producing a certain amount of TAG dependent on the amount of active enzyme present, mtGPAT1 activity is essential for controlling the partitioning of fatty-acyl CoAs to β-oxidation or glycerolipid synthesis.

Both mtGPAT1 and carnitine palmitoyltransferase-1 (CPT-1), the enzyme that catalyzes the rate-limiting step of β-oxidation, are located on the outer mitochondrial membrane. This suggests that there is a competition between these enzymes for fatty acyl-CoA substrates. AMP-activated protein kinase (AMPK), which inactivates acetyl-CoA carboxylase (ACC) by phosphorylation, appears to acutely regulate both of these enzymes. Inactivation of ACC by AMPK prevents the buildup of malonyl-CoA, an allosteric suppressor of CPT-1, resulting in an increase in β-oxidation. AMPK inhibits mtGPAT1 as well, thereby decreasing the amount of TAG produced. The relationship between these two processes has been demonstrated in vivo. Feeding mtGPAT1-knockout mice a high-fat, high-sugar diet to induce obesity resulted in an increase in oxidation as the long-chain acyl-CoA substrates were partitioned away from the TAG synthetic pathway toward CPT-1 and β-oxidation. MtGPAT1 overexpression in rat hepatocytes produced an 80% reduction in fatty acid oxidation coupled to an increase in phospholipid biosynthesis. Overexpression in vivo resulted in a decrease in β-oxidation as well.

The evidence suggesting that a drop in mtGPAT activity leads to a decrease in TAG levels as well as an increase in the amount of β-oxidation suggests that inhibition of this enzyme with a small molecule could be an effective treatment for obesity, diabetes, and other health problems associated with increased TAG synthesis. There is a need, therefore, for small molecules which can inhibit mtGPAT and other GPAT isoforms. Such compounds might be used for treating obesity or inducing weight loss.

### SUMMARY OF THE INVENTION

The present invention relates to a novel class of compounds comprising formula I:

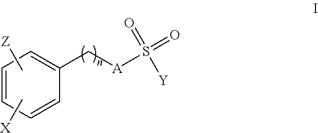

wherein n is either 0 or 1. A is selected from the group consisting of NR¹, O, and S, wherein R¹ is either a H, hydroxyl, C₁-C₁₀ alkyl, C₁-C₁₀ alkoxy, alkenyl, aryl, alkylaryl or arylalkyl. X is selected from the group consisting of a carboxylate residue, a phosphonate residue, a phosphate residue, or a C₁-C₁₀ alkyl residue which is optionally substituted with one or more carboxylate, phosphonate or phosphate residues. Y is selected from the group consisting of C₁-C₂₀ alkyl, alkenyl, halide, hydroxyl, C₁-C₂₀ alkoxy, aryl, alkylaryl, arylalkyl, cycloalkyl, cycloalkenyl, or a heterocyclic ring and may optionally be substituted at one or more positions with a halide. Z is selected from the group consisting of a H, a hydroxyl group, a halide, an aryl group, an alkylaryl group, an arylalkyl group, a cycloalkyl group, a cycloalkenyl group or a heterocyclic ring. In embodiments, where Z is an aryl group, an alkylaryl group, an arylalkyl group, a cycloalkyl group, a cycloalkenyl group or a heterocyclic ring, the ring moiety may be substituted with one or more substituent groups selected from a C₁-C₁₀ alkyl group, C₁-C₁₀ alkoxy group, a hydroxyl group, a cyano group, a carboxylate group, a halide, an aryl group, an alkylaryl group, an arylalkyl group, a cycloalkyl group, a cycloalkenyl group or a heterocyclic ring.

Based on the foregoing, one or more compounds of the present invention, either alone or in combination with another active ingredient, may be synthesized and administered as a therapeutic composition using dosage forms and routes of administration contemplated herein or otherwise known in the art. Dosaging and duration will further depend upon the factors provided herein and those ordinarily considered by one of skill in the art. To this end, determination of a therapeutically effective amounts are well within the capabilities of those skilled in the art, especially in light of the detailed disclosure and examples provided herein.

### DESCRIPTION OF THE FIGURES

FIG. 1 illustrates a first reaction scheme for manufacturing compounds of the instant invention, particularly compounds 5a-5d disclosed herein.

FIG. 2 illustrates a second reaction scheme for manufacturing compounds of the instant invention, particularly compounds 5e-5f disclosed herein.

FIG. 3 illustrates a third reaction scheme for manufacturing compounds of the instant invention, particularly compounds 13a-13f disclosed herein.

FIG. 4 illustrates a fourth reaction scheme for manufacturing compounds of the instant invention, particularly compounds 15a-15i disclosed herein.

FIG. 5 illustrates a fifth reaction scheme for manufacturing compounds of the instant invention, particularly compounds 17a-17f disclosed herein.

FIG. 6 illustrates a sixth reaction scheme for manufacturing compounds of the instant invention, particularly compounds 21a-21c disclosed herein.

FIG. 7 illustrates a first reaction scheme for manufacturing compounds of the instant invention, particularly compounds 24a-24f disclosed herein.

FIG. 8 illustrates a reaction scheme for manufacturing compounds 4a-t, disclosed herein.

FIG. 9 illustrates a reaction scheme for manufacturing compounds 7a-t.

FIG. 10 illustrates FSG67 inhibition of acylglyceride synthesis in 3T3-L1 adipocytes. The concentration dependent reduction of triglyceride synthesis is reflected in phase-contrast photomicrographs of cultured cells showing a corresponding reduction in lipid droplet accumulation (×400).

FIG. 11 illustrates acute FSG67 treatment of lean and DIO mice reduced body weight and decreased food consumption without conditioned taste aversion. Body weight and food intake were measured following a single 20 mg/kg ip dose of FSG67 in lean or DIO mice, 8 per group. (a) FSG67 treated lean mice (grey bar) lost 3.7±0.9% (1.0±0.2 g); fasted mice lost 15.5±0.7% (3.9±0.2 g) (black bar). The reduction in body mass of both treated and fasted mice was significant compared to the vehicle control mice (white bar) that gained 2.5±0.5% (0.6±0.1 g) (p<0.0001 2-tailed t-test). (b) FSG67 treatment reduced food consumption to 33% of vehicle control (1.4±0.2 g, grey bar, versus 4.2±0.2 g white bar, p<0.0001, 2-tailed t-test). (c) FSG67 treated DIO mice (grey bar) lost 4.3±0.5% (1.7±0.2 g) of body mass, fasted mice (black bar) lost 5.3±0.4% (2.1±0.2 g) and vehicle controls (white bar) lost a 2.5±0.6% (1.0±0.2 g). Compared to the vehicle controls, the weight loss was significant in both the FSG67 treated (p=0.026, 2-tailed t-test) and fasted (p=0.002, 2-tailed t-test) mice. (d) FSG67 reduced food consumption to 41.6% of vehicle control (0.5±0.1 g, grey bar versus 1.2±0.3 g, white bar, p=0.043, 2-tailed t-test). (e) FSG67 did not induce conditioned tasted aversion in mice. CTA testing using a two bottle choice paradigm in groups of 8 lean mice did not produce a significant reduction in saccharine intake at 5 mg/kg (p=0.12) or 20 mg/kg (p=0.10) (2-tailed t-tests). Thus, the FSG67 effect on food intake was likely a specific effect on appetite rather than an induction of sickness behavior. All data are expressed as means±SEM. (*, p<0.05; **, p<0.01; ***, p<0.001).

FIG. 12 illustrates chronic FSG67 treatment of DIO mice reversibly reduces body weight and food intake while enhancing fatty acid oxidation. (a) DIO mice, 4 per group, were treated with daily FSG67 5 mg/kg ip (red) or vehicle control (black) for 20 d (black arrow indicates termination of treatment) and were then allowed to regain their weight. The FSG67 treated mice lost 10.3±0.6% of body mass during treatment (days 0-19) compared to an increase of 4.0±0.5% for vehicle controls (p>0.0001, 2-way ANOVA analysis). The FSG67 weight loss was reversible with treated animals returning to original weight at day 32. (b) Food consumption was significantly reduced during FSG67 treatment (2.6±0.1 g/d) compared to vehicle controls (3.1±0.1 g/d) (p=0.0008, 2-way ANOVA). Following cessation of treatment at day 20, food consumption increased in the FSG67 treatment group to 3.5±0.1 g/d representing a significant increase in food intake compared to vehicle controls 3.2±0.1 g/d (p=0.006, 2-way ANOVA). (c) Following 3 days of acclimatization in the calorimeter, 8 DIO mice per group were treated with FSG67 5 mg/kg ip (red) or vehicle control (black) daily for 16 days, along with a group pair-fed to the FSG67 treated animals (blue). The FSG67-treated animals lost 9.5±0.6% and pair-fed lost 5.5±0.9% of body mass while the vehicle controls increased by 3.5±1.3%. The weight loss in the FSG67 treated animals was significant compared to both vehicle controls and pair-fed animals (p<0.0001, 2-way ANOVA). (d) FSG67 treatment (red) reduced average daily food consumption by 33% (2.0±0.1 g/d) compared to vehicle controls (black) 3.1±0.1 g/d (p<0.0001, 2-way ANOVA). (e) FSG67 treatment increased the average VO2 to 106.5±1.1% of the pre-treatment value (red line) compared to a reduction of 89.9±1.1% for the pair-fed group (blue line) (p<0.0001 2-way ANOVA) consistent with increased energy utilization. (f) In contrast, the average RER was lower for the FSG67 treated DIO mice (0.732±0.002) (red line) compared to (0.782±0.006) (blue line) for the pair-fed group (p<0.0001, 2-way ANOVA) indicating increased reliance on fatty acids for fuel.

FIG. 13 illustrates pharmacological GPAT inhibition reduced adiposity and down-regulated lipogenic gene expression in DIO mice. (a) Q-NMR analysis of FSG67 treated or vehicle control animals 10 per group. FSG67 treated animals (checkered bars) exhibited a significant reduction in fat mass (4.0 g) compared to vehicle controls (white bars) while lean and water mass were unaffected (p<0.0001, 2-tailed t-test). At the conclusion of the experiment, the vehicle control mice weighed 4.4 g more than the FSG67 controls (p=0.0014, 2-tailed t-test). (b) Real-time RT-PCR analysis of lipogenic gene expression in FSG67 treated (checkered bars), vehicle control (white bars), and pair-fed (black bars) DIO mice (from experiment shown in FIG. 4c). FSG67 reduced the expression of ACC1 (p=0.0005 vs. control, p=0.0004 vs. pair-fed), FAS (p=0.0001 vs. control, p=0.0007 vs. pair-fed), PPARγ (p=0.032 vs. control, p=0.0019 vs. pair-fed), and GPAT (p=0.0034 vs. control, p=0.0002 vs. pair-fed) were all down regulated in white adipose tissue. Data are analyzed with 2-tailed t-tests. (*, p<0.05; **, p<0.01; ***, p<0.001).

FIG. 14 illustrates FSG67 treatment reduced hepatic steatosis and serum triglyceride and glucose levels. Oil red-stained histological sections of liver from (a) vehicle control, (b) pair-fed, and (c) FSG67-treated DIO mice from the 16-day treatment experiment in FIG. 4. Note intracytoplasmic large and small droplet fat accumulation most prominent in the vehicle control (a). Pair feeding reduced steatosis, whereas FSG67-treated animals showed almost complete amelioration of fat accumulation. (d) Average serum triglyceride, cholesterol, and glucose measurements from vehicle control, FSG67-treated, and pair-fed mice from the same animals. FSG67-treated animals had significantly reduced serum glucose levels (153.3±10.5 mg/dL) compared to pair-fed mice (189.0±20.3 mg/dL, p=0.047) and vehicle controls (200.6±22.2 mg/dL, p=0.031 2-way ANOVA). The reduction in triglyceride levels were not statistically significant; cholesterol levels were unaffected. Data are expressed as means±SEM. (*, p<0.05; **, p<0.01; ***, p<0.001).

FIG. 15 illustrates Intracerebroventricular (icy) FSG67 treatment reduced food consumption and body weight. (a) FSG67 or vehicle was administered icy to groups of 6 lean mice. One day following treatment, mouse weight was significantly reduced by both 100 (vertical bars) and 320 nmole (checkered bar) doses (p=0.016, p=0.0003, 2-tailed t-tests). (b) A significant reduction in food intake occurred only in the 320 nmole group (checkered bar) (p=0.005, 2-tailed t-test). (*, p<0.05; **, p<0.01; ***, p<0.001).

FIG. 16 illustrates acute and chronic FSG67 treatment altered hypothalamic neuropeptide expression. (a) Real-time RT-PCR analysis of hypothalamic neuropeptides were conducted in lean mice treated with a single 20 mg/kg dose of FSG67 (from FIG. 3a). NPY was significantly reduced in the FSG67 treated group (grey bar) compared to fasted mice (black bar) (p=0.016), while AGRP expression was diminished compared to both vehicle control (white bar) (p=0.02) and fasted mice (p=0.0009). Expression of POMC and CART were unaffected. (b) Similar analysis from 16 d treated DIO mice (from FIG. 4c) showed a reduction of NPY expression in both FSG67 treated (p=0.0074) and pair-fed controls (p=0.0057). The expression of AGRP, POMC, and CART were unaffected. Data are analyzed with 2-tailed t-tests. (*, p<0.05; **, p<0.01; ***, p<0.001).

FIG. 17 illustrates dose response of FSG67 in DIO mice. Groups of DIO mice were treated daily with FSG67 ip at doses indicated or vehicle. Over the 5 day course, 5 mg/kg was the minimum dose that led to a significant weight loss of 3.9% compared to vehicle controls (p=0.008, 2-way ANOVA). (*p<0.05; **, p<0.01; ***, p<0.001).

FIG. 18 illustrates FSG67 treatment of DIO mice for Q-NMR analysis. DIO mice (10 per group) treated daily for 10 d with FSG67 (5 mg/kg) lost significant body mass (6.1 g, 13.1%) compared to vehicle controls (1.1 g, 2.4%) (p<0.0001, 2-way ANOVA).

FIG. 19 illustrates FSG67 treatment increases UCP2 expression in liver and WAT. Real-time RT-PCR expression analysis of LCPT-1 and UCP2 expression in liver and white adipose tissue. UCP2 expression was increased in the (a) liver (p=0.043 vs. control) and (b) WAT (p=0.013, vs. pair-fed) of DIO mice treated with FSG67 for 16 d (see FIG. 4C). L-CPT-1 expression was not affected by FSG67 treatment or pair-feeding. Data were analyzed with two-tailed t-tests, p<0.05; **, p<0.01; ***, p<0.001.

FIG. 20 illustrates FSG67 treatment down-regulated hepatic lipogenic genes. Real-time RT-PCR expression analysis of lipogenic gene expression in the liver of DIO mice treated with FSG67 for 16 d (see FIG. 12C). FAS expression was reduced compared to both vehicle and pair-fed animals (p=0.0016 vs. control, p=0.018 vs. pair-fed) while ACC1 was reduced compared to pair-fed animals (p=0.037). GPAT expression was unaffected. Data were analyzed with two-tailed t-tests, p<0.05; **, p<0.01; ***, p<0.001.

### DETAILED DESCRIPTION OF THE INVENTION

Definitions

As used herein, “an alkyl group” denotes both straight and branched carbon chains with one or more carbon atoms, but reference to an individual radical such as “propyl” embraces only the straight chain radical, a branched chain isomer such as “isopropyl” specifically referring to only the branched chain radical. A “substituted alkyl” is an alkyl group wherein one or more hydrogens of the alkyl group are substituted with one or more substituent groups as otherwise defined herein.

As used herein, “an alkoxy group” refers to a group of the formula alkyl-O—, where alkyl is as defined herein. A “substituted alkoxy” is an alkoxy group wherein one or more hydrogens are substituted with one or more of the substitutent groups otherwise defined herein.

As used herein, “alkenyl” refers to a partially unsaturated alkyl radical derived by the removal of one or more hydrogen atoms from a alkyl chain such that it contains at least one carbon-carbon double bond.

As used herein, “an aryl group” denotes a structure derived from an aromatic ring containing six carbon atoms. Examples include, but are not limited to a phenyl or benzyl radical and derivatives thereof.

As used herein, “arylalkyl” denotes an aryl group having one or more alkyl groups not at the point of attachment of the aryl group.

As used herein, “alkylaryl” denotes an aryl group having an alkyl group at the point of attachment.

A used herein, “carboxylate” denotes salt or ester of an organic acid, containing the radical —COOR, wherein R may be, but is not limited to, a H, an alkyl group, an alkenyl group, or any other residue otherwise known in the art.

As used herein, “carboxylic acid” denotes an organic functional group comprising the following structure: —COOH or —CO₂H.

As used herein, “cyano” denotes an organic functional group comprising the following structure: —C≡N.

As used herein, “cycloalkyl” refers to a monovalent or polycyclic saturated or partially unsaturated cyclic non-aromatic group containing all carbon atoms in the ring structure, which may be substituted with one or more substituent groups defined herein. In certain non-limiting embodiments the number of carbons comprising the cycloalkyl group may be between 3 and 7.

As used herein, “cycloalkenyl” refers to a partially unsaturated cycloalkyl radical derived by the removal of one or more hydrogen atoms from a cycloalkyl ring system such that it contains at least one carbon-carbon double bond.

As used herein, “halogen” or “halide” denotes any one or more of a fluorine, chlorine, bromine, or iodine atoms.

As used herein, “heterocyclic” refers to a monovalent saturated or partially unsaturated cyclic aromatic or non-aromatic carbon ring group which contains at least one heteroatom, in certain embodiments between 1 to 4 heteroatoms, which may be but is not limited to one or more of the following: nitrogen, oxygen, sulfur, phosphorus, boron, chlorine, bromine, or iodine. In further non-limiting embodiments, the hetercyclic ring may be comprised of between 1 and 10 carbon atoms.

As used herein, “hydroxyl” denotes an organic functional group comprising the following structure: —OH.

As used herein, “phosphonate” denotes an organic functional group comprising the following structure: —PO₃H₂ or —PO(OH)₂.

As used herein, “phosphate” denotes an organic functional group comprising the following structure: —OPO₃H₂ or —OPO(OH)₂.

The present invention relates to novel compounds, pharmaceutical compositions containing the same, and methods of use by inhibiting the enzymatic activity of Glycerol 3-phosphate acyltransferase (GPAT). Such compounds, compositions, and methods have a variety of therapeutically valuable uses including, but not limited to, treating obesity. The class of compounds of the present invention are comprised of formula I:

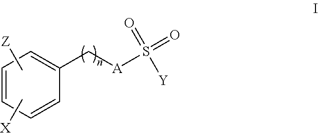

wherein n is either 0 or 1. A is selected from the group consisting of NR¹, O, and S, wherein R¹ is either a H, hydroxyl, C₁-C₁₀ alkyl, C₁-C₁₀ alkoxy, alkenyl, aryl, alkylaryl or arylalkyl. X is selected from the group consisting of a carboxylate residue, a phosphonate residue, a phosphate residue, or a C₁-C₁₀ alkyl residue which is optionally substituted with one or more carboxylate, phosphonate or phosphate residues. Y is selected from the group consisting of C₁-C₂₀ alkyl, alkenyl, halide, hydroxyl, C₁-C₂₀ alkoxy, aryl, alkylaryl, arylalkyl, cycloalkyl, cycloalkenyl, or a heterocyclic ring. In embodiments where Y is a C₁-C₂₀ alkyl, alkenyl, C₁-C₂₀ alkoxy, aryl, alkylaryl, arylalkyl, cycloalkyl, cycloalkenyl, or a heterocyclic ring, it is optionally substituted at one or more positions with a halide. Z is selected from the group consisting of a H, a hydroxyl group, a halide, an aryl group, an alkylaryl group, an arylalkyl group, a cycloalkyl group, a cycloalkenyl group or a heterocyclic ring. In embodiments, where Z is an aryl group, an alkylaryl group, an arylalkyl group, a cycloalkyl group, a cycloalkenyl group or a heterocyclic ring, the ring moiety may be substituted with one or more substituent groups selected from a C₁-C₁₀ alkyl group, C₁-C₁₀ alkoxy group, a hydroxyl group, a cyano group, a carboxylate group, a halide, an aryl group, an alkylaryl group, an arylalkyl group, a cycloalkyl group, a cycloalkenyl group or a heterocyclic ring.

In certain embodiments, X is comprised of either a carboxylic acid residue or a phosphonate residue. In alternative embodiments, X may include a C₁-C₁₀ alkyl group, which is substituted at one or more positions with either a phosphonate residue or carboxylate. In further embodiments, the alkyl group may comprise between 1 and 3 carbons. In any of the foregoing, X may be positioned on the phenyl ring in either the ortho, meta, or para position with respect to the sulfonyl linker. As shown below, in certain non-limiting embodiments X occupies either the ortho or meta position.

In further non-limiting embodiments, Y is comprised of a C₁-C₂₀ alkyl group, which may be either a CH₃, C₅H₁₁, C₈H₁₇, C₉H₁₉, C₁₄H₂₉, an Alternatively, Y may be comprised of an aryl ring system, which is optionally substituted with one or more halogen atoms. In even further alternative embodiments, Y is comprised of an alkylaryl residue, wherein the alkyl moiety connects the aryl ring to the Y position. The alkyl chain may have between 1 to 3 carbon atoms, with certain embodiments having 1 or 2 carbon atoms. The aryl residue in this latter embodiment may be substituted with one or more halogen atoms.

In even further non-limiting embodiments, Z is either a hydrogen atom, a hydroxyl group, a halogen atom, an optionally substituted aryl group or an optionally substituted heterocyclic ring. In any of the foregoing, Z may be position on the phenyl ring in either the ortho, meta, or para position with respect to the sulfonyl linker. As shown below, in certain non-limiting embodiments Z occupies either the meta or para position with respect to the sulfonyl linker of the phenyl ring. In even further embodiments, Z occupies either the meta or para position with respect to both the sulfonyl linker and X positions.

Based on the foregoing, one compound of the instant invention is C-67 or FSG67 and is comprised of the following structure:

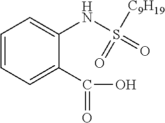

In another embodiment, the compounds of the instant invention may be comprised of the following structures:

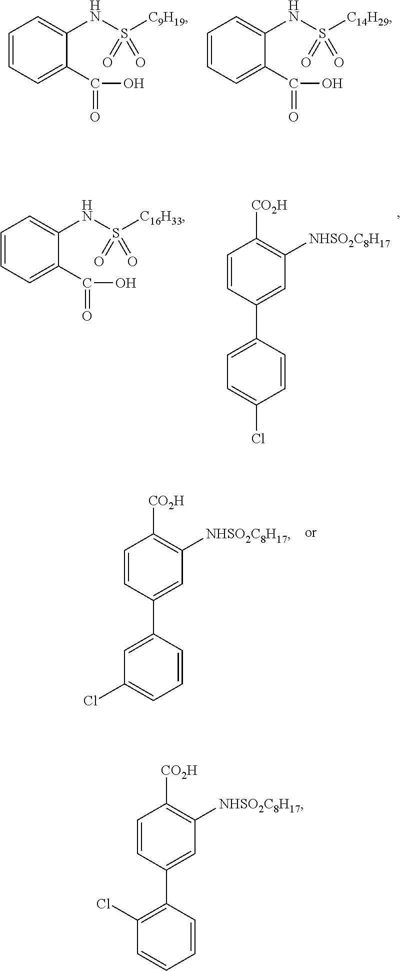

In an even further embodiment, the compounds of the instant invention may be comprised of one or more of the following:

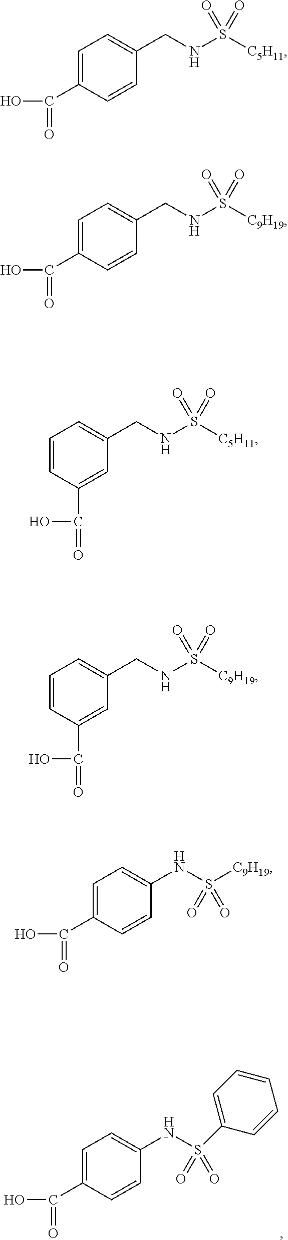

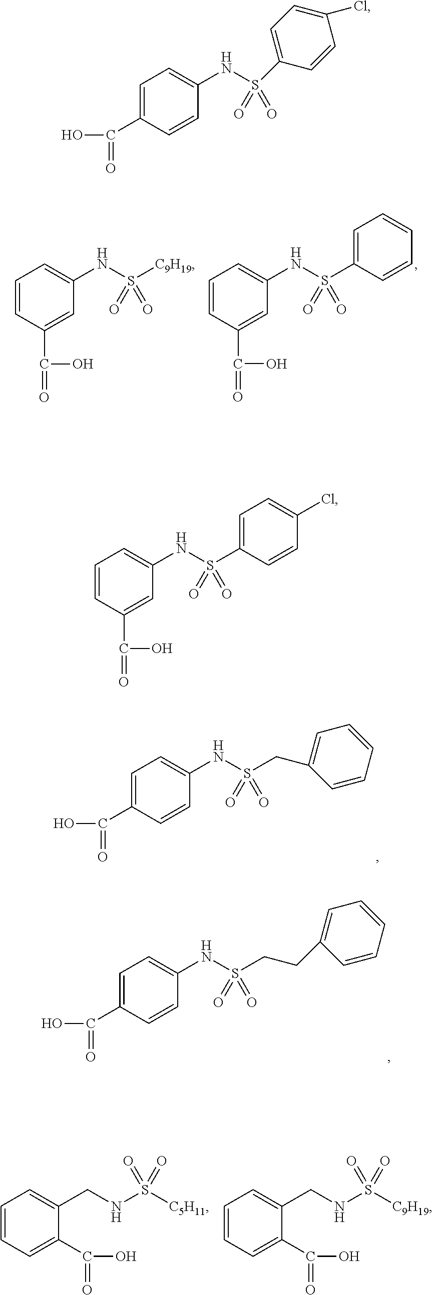

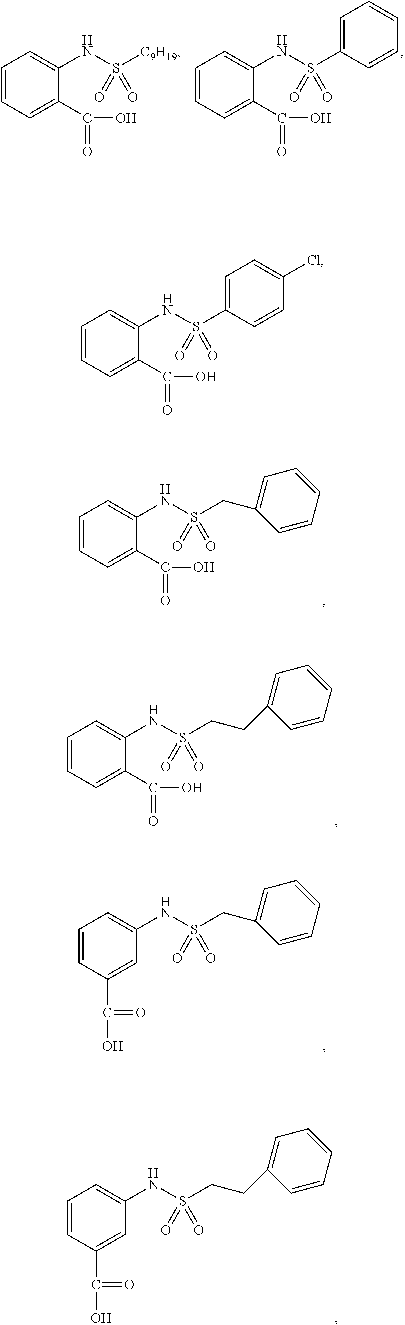

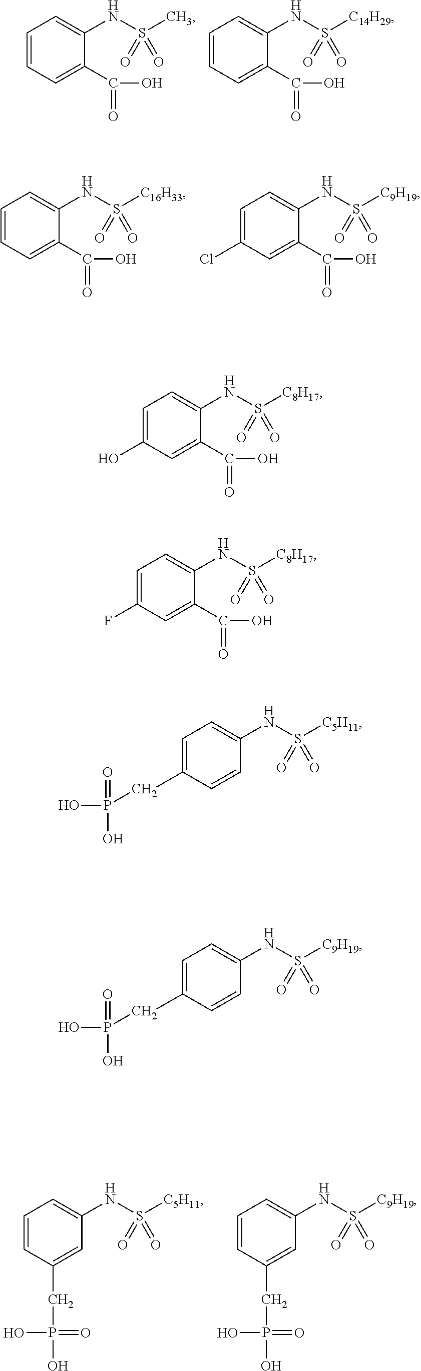

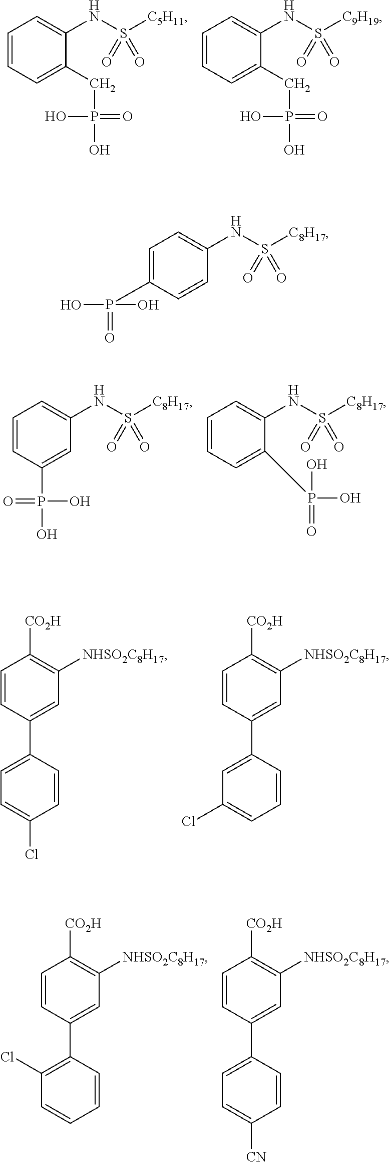

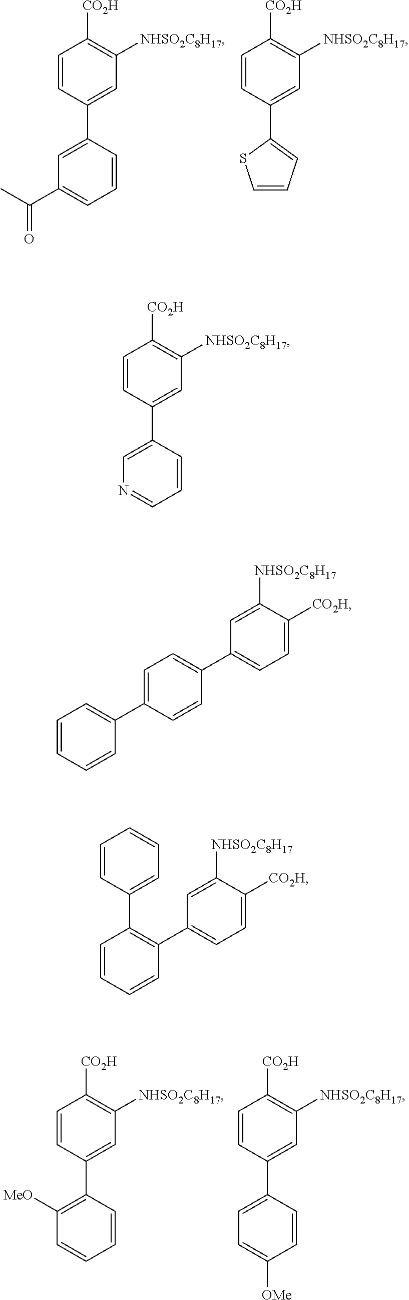

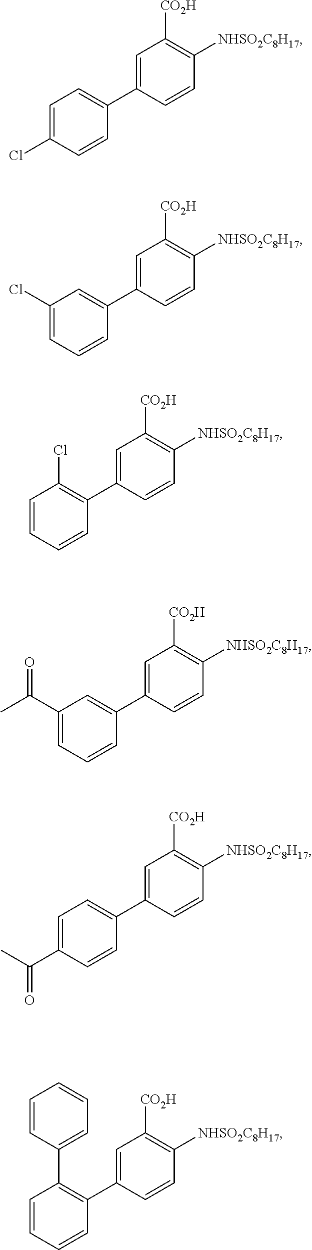

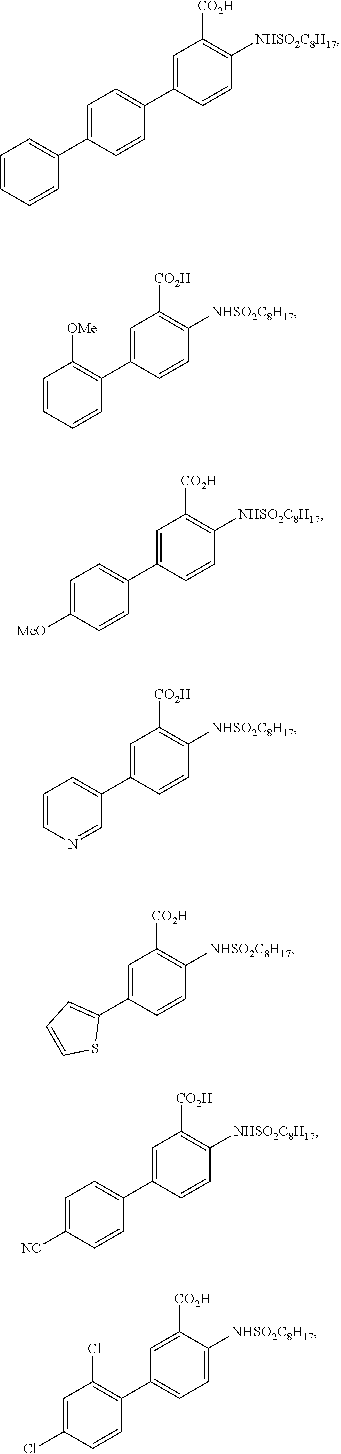

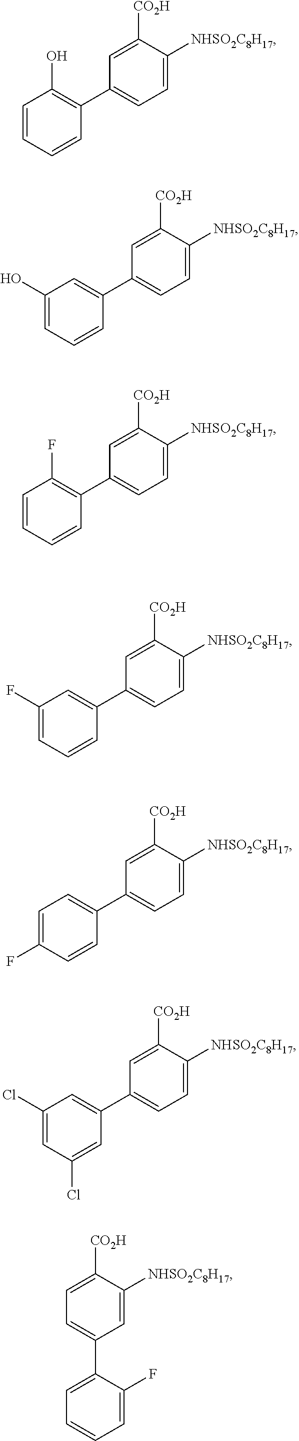

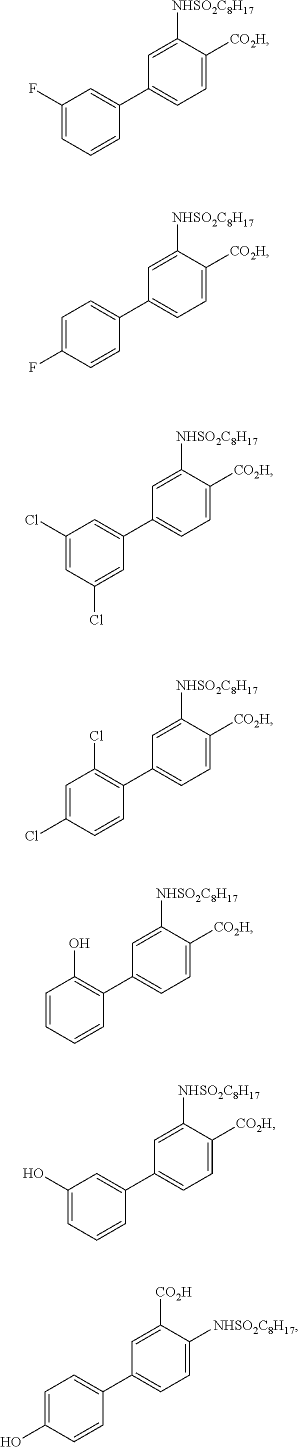

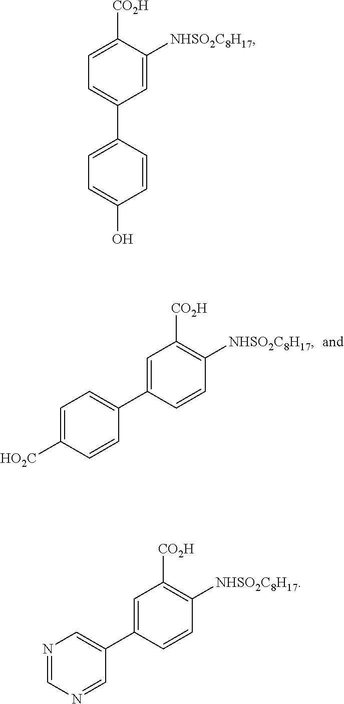

Based on the foregoing, in certain non-limiting embodiments of formula I, A is comprised of NR¹ wherein R¹ is any of the embodiments defined above. In further embodiments R¹ is a hydrogen atom. To this end, certain embodiments of the compounds of the instant invention may be represented by formula II:

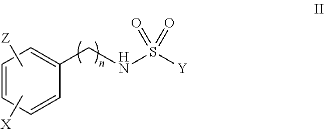

wherein each of n, X, Y, and Z are any of the embodiments defined above.

In alternative embodiments of formula I, n is comprised of 0. To this end, certain compounds of the instant invention may be represented by formula III:

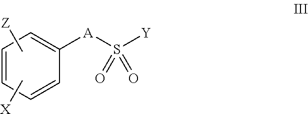

wherein each of A, X, Y, and Z are any of the embodiments defined above.

In even further embodiments of formula I, X is comprised of a carboxylic acid residue at either the ortho, meta or para positions with respect to the sulfonyl linker of the phenyl ring. Accordingly, certain compounds of the instant invention may be represented by formula IVa:

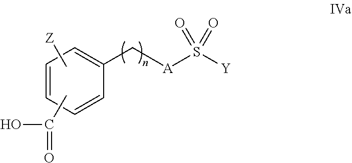

wherein each of n, A, Y, and Z are any of the embodiments defined above.

While the carboxylic acid residue may occupy either the ortho, meta, or para positions, in certain embodiments it occupies the ortho position with respect to the sulfonyl linker. To this end, certain compounds of the instant invention may be represented by formula IVb:

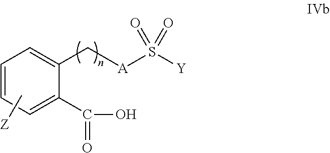

wherein each of n, A, Y, and Z are any of the embodiments defined above.

Similarly, although it may occupy either the ortho, meta, or para positions, in certain compounds of the instant invention Z occupies either the meta or the para postions with respect to both the sulfonyl linker and X, as set forth below in formulas IVc and IVd:

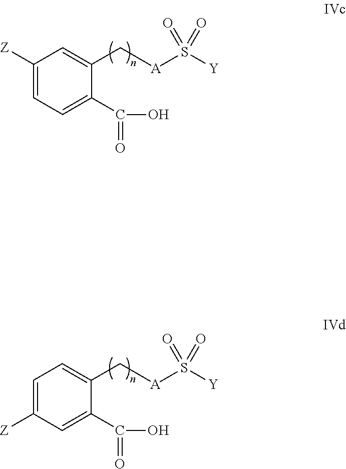

wherein each of n, A, Y, and Z are any of the embodiments defined above.

Based on the foregoing structures of formulas IVc-d compounds of the instant invention may be comprised of one or more of the following:

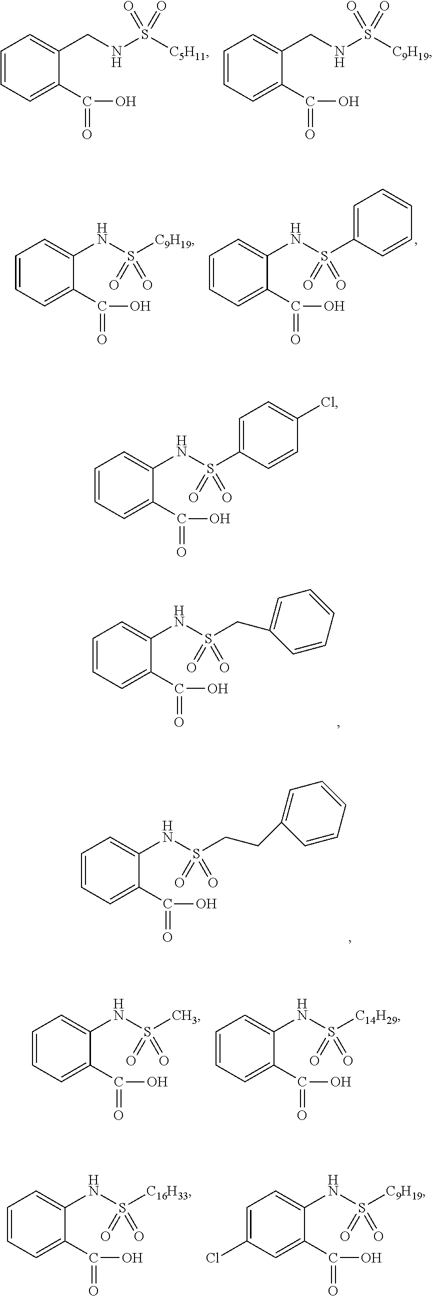

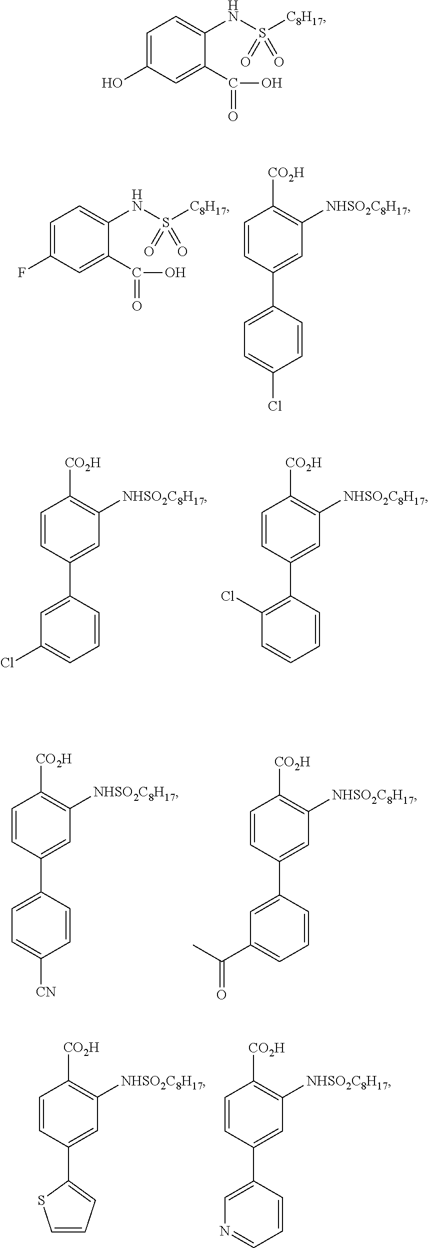

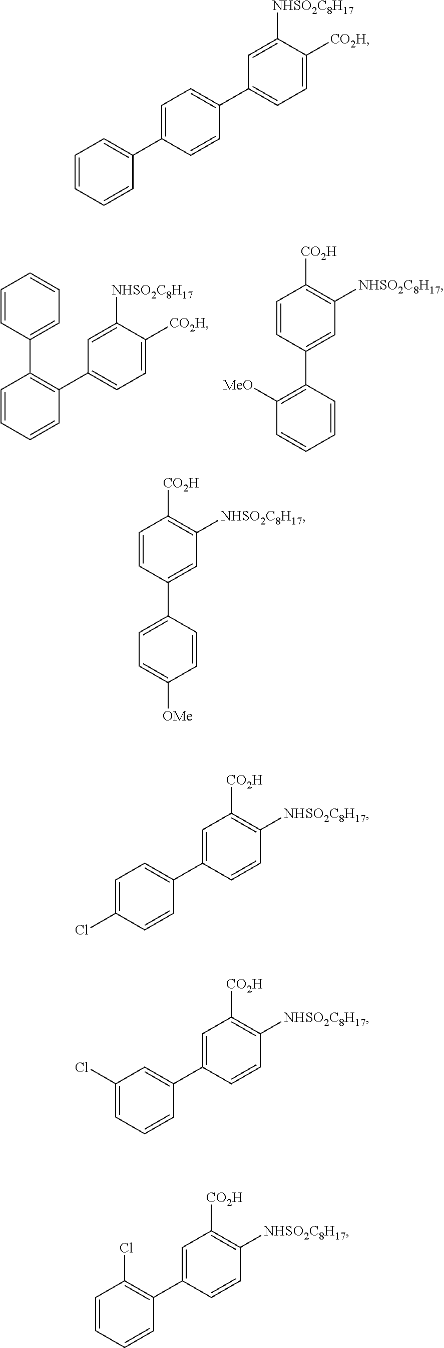

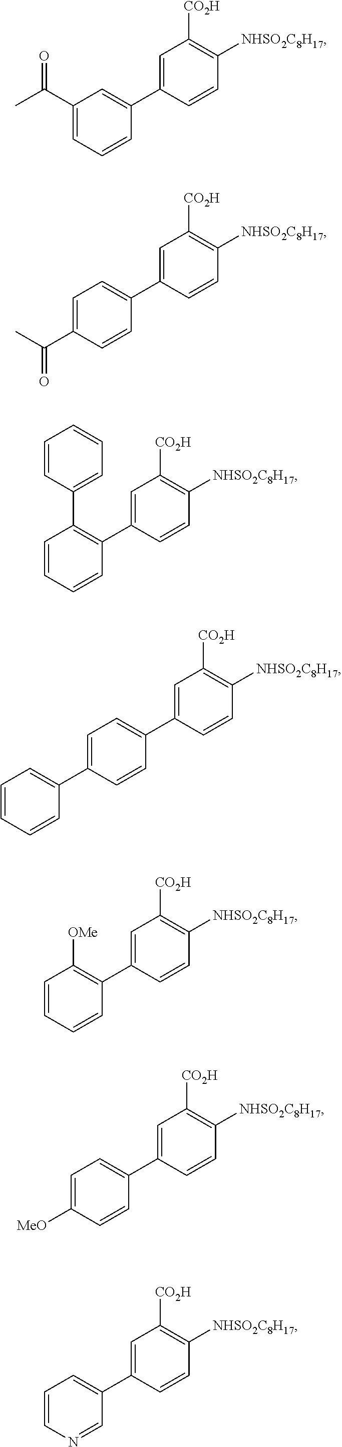

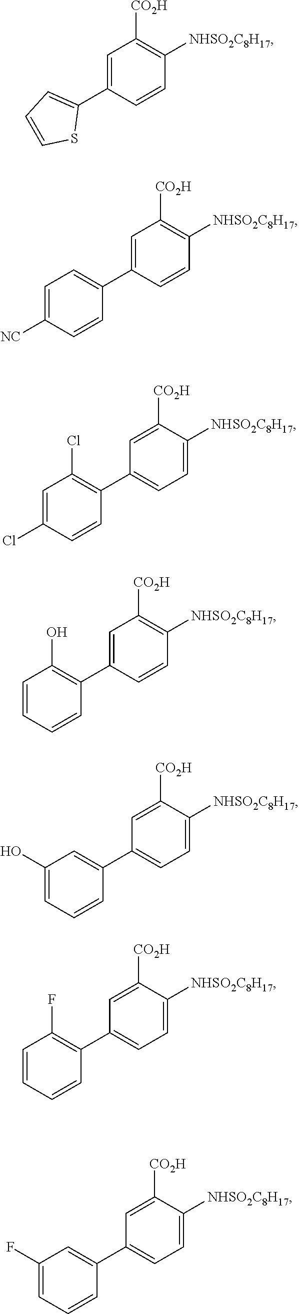

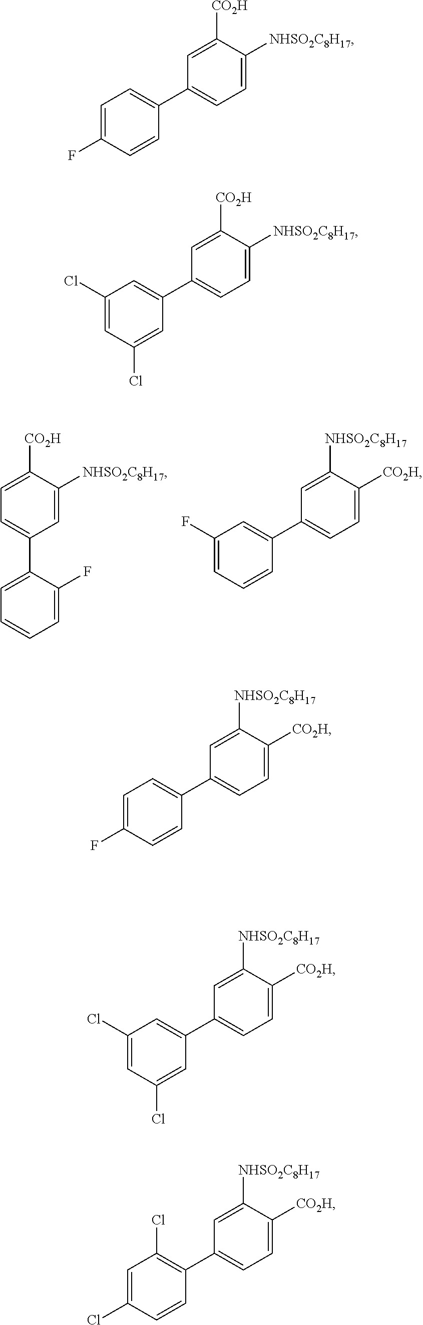

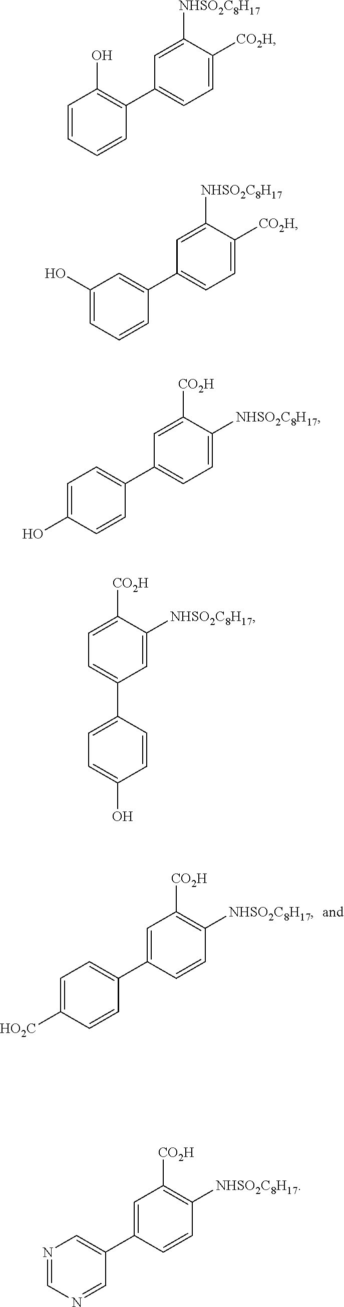

In further embodiments of formula I, X is comprised of either a phosphate group or an alkyl residue having 1 to 3 carbon atoms, which is substituted with a phosphonate group. Such compounds of the instant invention may be represented by formula V:

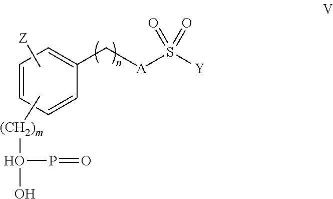

wherein m is comprised of either 0, 1, 2, or 3 and each of n, A, Y and Z are any of the embodiments defined above.

Accordingly, compounds of the instant invention may be comprised of one or more of the following:

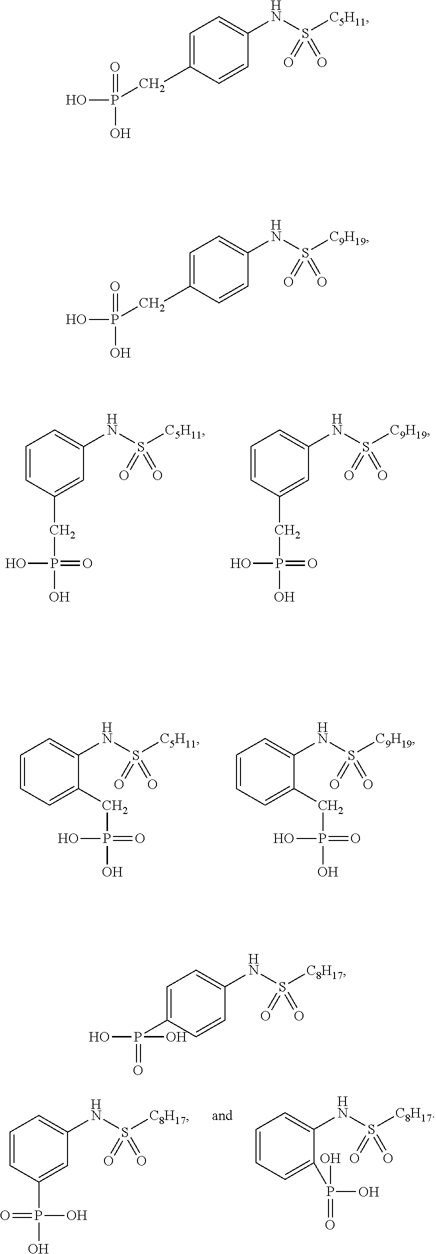

Without seeking to limit the possible scope of use of the foregoing compounds, the clinical therapeutic indications envisioned include, but are not limited to, treatment of obesity or the induction of weight loss. One or more small molecules, or pharmaceutical salts thereof, of the present invention may be synthesized and administered as a composition used to treat and/or prevent obesity by targeted GPAT activity, in particular mtGPAT activity, and/or by stimulating fatty acid oxidation. Compounds of the present invention may be synthesized using methods known in the art or as otherwise specified herein.

Unless otherwise specified, a reference to a particular compound of the present invention includes all isomeric forms of the compound, to include all diastereomers, tautomers, enantiomers, racemic and/or other mixtures thereof. Unless otherwise specified, a reference to a particular compound also includes ionic, salt, solvate (e.g., hydrate), protected forms, and prodrugs thereof. To this end, it may be convenient or desirable to prepare, purify, and/or handle a corresponding salt of the active compound, for example, a pharmaceutically-acceptable salt. Examples of pharmaceutically acceptable salts are discussed in Berge et al., 1977, “Pharmaceutically Acceptable Salts,” J. Pharm. Sci., Vol. 66, pp. 1-19, the contents of which are incorporated herein by reference.

Based on the foregoing, one or more compounds of the present invention, either alone or in combination with another active ingredient, may be synthesized and administered as a therapeutic composition. The compositions of the present invention can be presented for administration to humans and other animals in unit dosage forms, such as tablets, capsules, pills, powders, granules, sterile parenteral solutions or suspensions, oral solutions or suspensions, oil in water and water in oil emulsions containing suitable quantities of the compound, suppositories and in fluid suspensions or solutions. To this end, the pharmaceutical compositions may be formulated to suit a selected route of administration, and may contain ingredients specific to the route of administration. Routes of administration of such pharmaceutical compositions are usually split into five general groups: inhaled, oral, transdermal, parenteral and suppository. In one embodiment, the pharmaceutical compositions of the present invention may be suited for parenteral administration by way of injection such as intravenous, intradermal, intramuscular, intrathecal, or subcutaneous injection. Alternatively, the composition of the present invention may be formulated for oral administration as provided herein or otherwise known in the art.

As used in this specification, the terms “pharmaceutical diluent” and “pharmaceutical carrier,” have the same meaning. For oral administration, either solid or fluid unit dosage forms can be prepared. For preparing solid compositions such as tablets, the compound can be mixed with conventional ingredients such as talc, magnesium stearate, dicalcium phosphate, magnesium aluminum silicate, calcium sulfate, starch, lactose, acacia, methylcellulose and functionally similar materials as pharmaceutical diluents or carriers. Capsules are prepared by mixing the compound with an inert pharmaceutical diluent and filling the mixture into a hard gelatin capsule of appropriate size. Soft gelatin capsules are prepared by machine encapsulation of a slurry of the compound with an acceptable vegetable oil, light liquid petrolatum or other inert oil.

Fluid unit dosage forms or oral administration such as syrups, elixirs, and suspensions can be prepared. The forms can be dissolved in an aqueous vehicle together with sugar or another sweetener, aromatic flavoring agents and preservatives to form a syrup. Suspensions can be prepared with an aqueous vehicle with the aid of a suspending agent such as acacia, tragacanth, methylcellulose and the like.

For parenteral administration fluid unit dosage forms can be prepared utilizing the compound and a sterile vehicle. In preparing solutions the compound can be dissolved in water for injection and filter sterilized before filling into a suitable vial or ampoule and sealing. Adjuvants such as a local anesthetic, preservative and buffering agents can be dissolved in the vehicle. The composition can be frozen after filling into a vial and the water removed under vacuum. The lyophilized powder can then be scaled in the vial and reconstituted prior to use.

Dose and duration of therapy will depend on a variety of factors, including (1) the patient's age, body weight, and organ function (e.g., liver and kidney function); (2) the nature and extent of the disease process to be treated, as well as any existing significant co-morbidity and concomitant medications being taken, and (3) drug-related parameters such as the route of administration, the frequency and duration of dosing necessary to effect a cure, and the therapeutic index of the drug. In general, the dose will be chosen to achieve serum levels of 1 ng/ml to 100 ng/ml with the goal of attaining effective concentrations at the target site of approximately 1 gg/ml to 10 μg/ml. Using factors such as this, a therapeutically effective amount may be administered so as to ameliorate the targeted symptoms of and/or treat or prevent obesity or diseases related thereto. Determination of a therapeutically effective amount is well within the capabilities of those skilled in the art, especially in light of the detailed disclosure and examples provided herein.

### EXAMPLES

### Example 1

### Chemical Syntheses of compounds 5a-5d

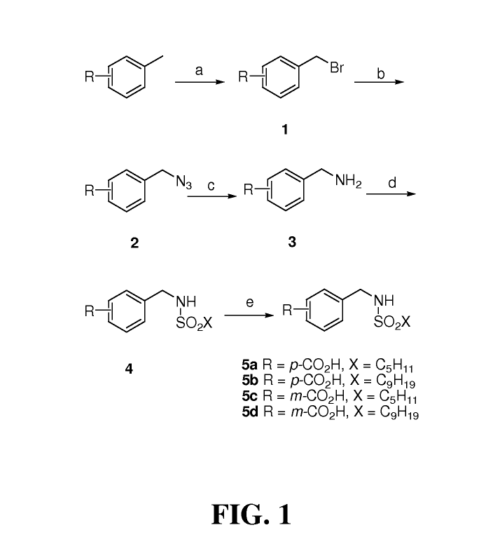

Synthesis of compounds 5a-5d was performed using Scheme 1, as illustrated in FIG. 1 herein.

Reaction conditions: (a) NBS, hv, CH₃CN; (b) NaN₃, EtOH, reflux; (d) C₉H₁₉SO₂Cl or C₅H₁₁SO₂Cl, pyridine, CH₂Cl₂, 0° C. to room temperature; (e) K⁺O⁻t-Bu, Et₂O, H₂O, 0° C. to room temperature.

The first series of compounds was derived from the variously substituted methyl methylbenzoates. The meta- and para-amines were made by following a literature protocol. (Okada, Y. et al., Bromination by means of sodium monobromoisocyanurate (SMBI). Org. Biomolec. Chem. 2003, 1, 2506-2511.) Following radical bromination of the methyl group with NBS in CH₃CN, the bromide was displaced by refluxing with NaN₃ in EtOH. Under Staudinger conditions, the azide was reduced to the free amine 3, which could then be coupled to 1-pentane- or 1-nonanesulfonyl chloride, prepared as described. (Blotny, G., A new, mild preparation of sulfonyl chlorides, Tet. Lett. 2003, 44, 1499-1501.) Finally, the methyl ester 4 was converted to the carboxylate product 5 by reaction with potassium t-butoxide in Et₂O with water present.

General Procedure for 4a-d. To a stirring solution of the appropriate amine 3a-c (1.2 mmol) in CH₂Cl₂ (4 mL) at 0° C., the sulfonyl chloride (1.3 mmol) was added dropwise, followed by Et₃N (1.3 mmol). The reaction mixture was allowed to warm to room temperature, where it was stirred for 2-3 h. Saturated NH₄Cl solution was added to quench the reaction, and the mixture was extracted with 3×10 mL CH₂Cl₂. The combined organic layers were dried over MgSO₄, concentrated in vacuo, and the products were purified by flash chromatography (20% EtOAc in hexanes).

General Procedure for 5a-d. To a stirring suspension of potassium t-butoxide (5.88 mmol) in Et₂O (15mL) cooled to 0° C., was added water (1.4 mmol) via syringe. The slurry was stirred for 5 min, and 4a-d (0.67 mmol) was added. The mixture was stirred at room temperature until starting material disappeared by TLC analysis (20% EtOAc in hexanes). Ice water was added until 2 clear layers formed. The aqueous layer was separated and acidified with 1 M HCl. The product was then extracted with Et₂O (3×20 mL) and evaporated in vacuo to afford 5a-d.

4-(Pentylsulfonamidomethyl)benzoic acid 5a. mp=188-189° C.; ¹H NMR (MeOD) δ 8.02 (d, J=8.4 Hz, 2H), 7.50 (d, J=8.1 Hz, 2H), 4.31 (s, 2H), 2.95 (t, J=8.1 Hz, 2H), 1.73 (m, 2H), 1.33 (m, 4H), 0.91 (t, J=6.9 Hz, 3H); ¹³C NMR (MeOD) δ 169.5, 145.0, 131.1, 131.0, 128.8, 53.6, 47.2, 31.4, 24.3, 23.2, 14.0; HRMS (FAB) calcd for C₁₃H₂₀NO₄S [M+H]⁺, 286.11131; found, 286.1111.

4-(Nonylsulfonamidomethyl)benzoic acid 5b. mp=178-180° C.; ¹H NMR (MeOD) δ 8.03 (d, J=8.4 Hz, 2H), 7.51 (d, J=8.1 Hz, 2H), 4.32 (s, 2H), 2.94 (t, J=7.8 Hz, 2H), 1.71 (m, 2H), 1.30 (m, 12H), 0.92 (t, J=6.9 Hz, 3H); ¹³C NMR (DMSO-d₆) δ 167.0, 143.6, 129.5, 129.3, 127.5, 51.5, 45.4, 31.2, 28.6, 28.5, 28.4, 27.4, 23.0, 22.0, 13.9; HRMS (FAB) calcd for C₁₇H₂₈NO₄S [M+H]⁺, 342.17391; found, 342.17447.

3-(Pentylsulfonamidomethyl)benzoic acid 5c. mp=160-161° C. ; ¹H NMR (MeOD) δ 8.08 (s, 1H), 7.97 (d, J=7.8 Hz, 1H), 7.63 (d, J=7.8 Hz, 1H), 7.48 (t, J=7.8 Hz, 1H), 4.31 (s, 2H), 2.92 (t, J=8.1 Hz, 2H), 1.72 (m, 2H), 1.33 (m, 4H), 0.91 (t, J=6.9 Hz, 3H); ¹³C NMR (MeOD) δ 169.5, 140.2, 133.5, 132.3, 130.1, 129.8, 129.7, 53.6, 47.1, 31.4, 24.3, 23.1, 14.0; HRMS (FAB) calcd for C₁₃H₁₈NO₃S [M-OH]⁺, 268.10074; found, 268.09988.

3-(Nonylsulfonamidomethyl)benzoic acid 5d. mp=150-151° C; ¹H NMR (MeOD) δ 8.08 (s, 1H), 7.97 (d, J=7.6 Hz, 1H), 7.63 (d, J=7.6 Hz, 1H), 7.48 (t, J=7.6 Hz, 1H), 4.31 (s, 2H), 2.91 (t, J=8.0 Hz, 2H), 1.70 (m, 2H), 1.28 (m, 12H), 0.92 (t, J=7.2 Hz); ¹³C NMR (MeOD) δ 169.5, 140.2, 133.5, 132.3, 130.1, 129.9, 129.7, 53.7, 47.2, 32.9, 30.4, 30.3, 30.1, 29.2, 24.6, 23.6, 14.4; HRMS (FAB) calcd for C₁₇H₂₈NO₄S [M+H]⁺, 342.17391; found, 342.17333.

### Example 2

### Synthesis of Compounds 5e and 5f

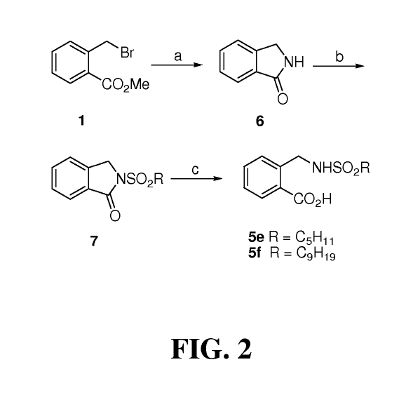

Synthesis of compounds 5e-5f was performed using Scheme 2, as illustrated in FIG. 2 herein.

Reaction conditions: (a) NH₃, MeOH, reflux; (b) NaH, RSO₂Cl, DMF, 0° C. to room temperature; (c) NaOH, THF/H₂O, 0° C. to room temperature.

The ortho-substituted carboxylates required a different approach than the meta- and para-compounds. Indolinone 6, formed in a reaction between the ortho-bromide and ammonia gas in MeOH, (Kovtunenko, V. A., et al.; Condensation of o-(bromomethyl)benzoic acid with amines, Ukrainskii Khimicheskii Zhurnal 1983, 49, 1099-1103) was coupled to the alkane sulfonyl chlorides with NaH in DMF, and the resulting γ-lactam bond was readily cleaved with NaOH in THF/H₂O to produce carboxylic acids 5e and 5f.

General Procedure for 7a-b. 1.5 mmol 6 was added to DMF (8 mL), and the solution was cooled to 0° C. NaH (1.65 mmol) was added, followed by the sulfonyl chloride (1.8 mmol), and the mixture was stirred and allowed to warm to room temperature. Reaction progress was monitored by TLC (25% MeOH in CHCl₃). When complete, saturated ammonium chloride solution was added (80 mL), the product was extracted with EtOAc (3×20 mL), dried over MgSO₄, and evaporated in vacuo. The product was purified by flash chromatography (2% MeOH in CHCl₃).

General Procedure for 5e-f. 7a-b (0.66 mmol) was dissolved in THF (3 mL), and the solution was cooled to 0° C. 1 M NaOH (1 mL, 10 equiv) was then added, and the solution was stirred and warmed to room temperature. Reaction progress was monitored by TLC (1:1 EtOAc:hexanes). When starting material had completely reacted, saturated NaHCO₃ (30 mL) was added, and the solution was washed with EtOAc. The aqueous phase was acidified to pH 3 with 1 M HCl, and product was extracted with EtOAc, dried over MgSO₄, and evaporated in vacuo.

2-(Pentylsulfonamidomethyl)benzoic acid 5e. mp=100° C.; ¹H NMR (DMSO-d₆) δ 13.0 (s, 1H), 7.87 (d, J=8.1 Hz, 1H), 7.60 (m, 2H), 7.39 (m, 2H), 4.51 (d, J=6.3 Hz, 2H), 2.92 (t, J=7.8 Hz, 2H), 1.61 (m, 2H), 1.25 (m, 4H), 0.84 (t, J=6.9 Hz, 3H); ¹³C NMR (MeOD) δ 170.3, 140.8, 133.6, 132.3, 131.3, 130.7, 128.8, 53.6, 46.5, 31.3, 24.3, 23.1, 14.0; HRMS (FAB) calcd for C₁₃H₂₀NO₄S [M+H]⁺, 286.11131; found, 286.11103.

2-(Nonylsulfonamidomethyl)benzoic acid 5f. mp=79-82° C.; ¹H NMR (CDCl₃) δ 8.04 (d, J=7.2 Hz, 1H), 7.60 (m, 2H), 7.43 (t, J=6.8 Hz, 1H), 4.60 (s, 2H), 2.89 (t, J=8.0 Hz, 2H), 1.66 (m, 2H), 1.28 (m, 12H), 0.92 (t, J=7.2 Hz, 3H); ¹³C NMR (DMSO-d₆) δ 168.3, 139.7, 132.1, 130.4, 129.8, 129.0, 127.2, 51.7, 44.1, 31.3, 28.7, 28.6, 28.5, 27.6, 23.1, 22.1, 14.0; HRMS (FAB) calcd for C₁₇H₂₈NO₄S [M+H]⁺, 342.17391; found, 342.17478.

### Example 3

### Synthesis of Compounds 13a-13f

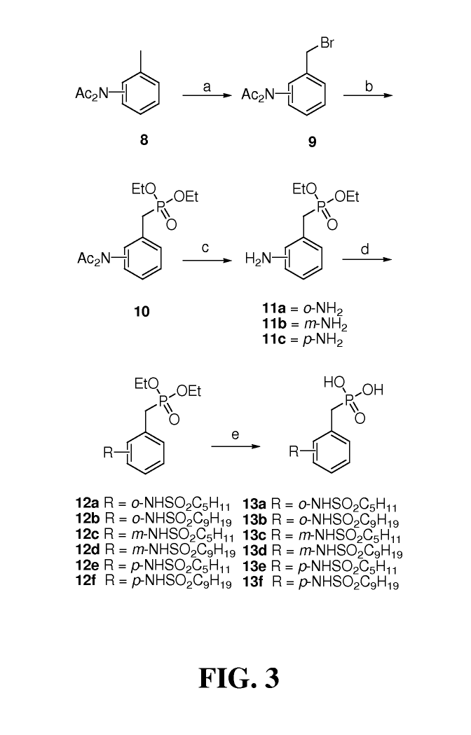

Synthesis of compounds 13a-13f was performed using Scheme 3, as illustrated in FIG. 3 herein.

Reaction conditions: (a) NBS, hv, CH₃CN; (b) P(OEt)₃, reflux; (c) H₂SO₄, EtOH, reflux; (d) C₉H₁₉SO₂Cl or C₅H₁₁SO₂Cl, pyridine, CH₃CN, 0° C. to room temperature; (e) TMSBr, CH₂Cl₂, room temperature.

The synthesis of the alkyl phosphonates 13a-f commenced with the protection of the starting toluidines as the bis-acylated aniline 8 (Brown, J. J.; Brown, R. K. Preparation of o- and p-acetamidobenzaldehydes, Can. J. Chem. 1955, 33, 1819-1823). Free-radical bromination with NBS in CH₃CN afforded benzyl bromide 9, which was converted to phosphonate 10 through Arbuzov reaction with triethyl phosphite. The aniline was unmasked by exposure to a refluxing acidic solution of EtOH. Following coupling of the amine with the alkane sulfonyl chloride to produce sulfonamide 12, the phosphonic acid moiety was revealed by treatment with TMSBr in CH₂Cl₂ followed by methanolysis.

General Procedure for 9a-c. 8a-c (31.3 mmol) was dissolved in CH₃CN (150 mL) and NBS (31.3 mmol) was added. The solution was then heated to reflux with a 275 W Sunlamp. Reaction progress was monitored by TLC (30% EtOAc in hexanes). The solution was then cooled, evaporated in vacuo, and the mixture was purified by flash chromatography (30% EtOAc in hexanes).

General Procedure for 10a-c. 9a-c (22.2 mmol) was dissolved in P(OEt)₃ (25 mL, 6.6 equiv), and the solution was heated to reflux for 18 h with a reflux condenser heated to 50° C. Reaction progress was monitored by TLC (30% EtOAc in hexanes). The reaction mixture was then cooled, and P(OEt)₃ was removed in vacuo. The product was then purified by flash chromatography (2% MeOH in CHCl₃).

General Procedure for 11a-c. Concentrated H₂SO₄ (3 mL) was added to a stirring solution of 10a-c (9.7 mmol) in EtOH (60 mL). The solution was heated to reflux for 18 h. Reaction progress was monitored by TLC (5% MeOH in CHCl₃). The solution was diluted with water (100 mL), washed with EtOAc (30 mL), and the aqueous phase was brought to pH 9 with saturated NaHCO₃ solution. The product was extracted with EtOAc (3×30 mL), the combined organic layers were dried over MgSO₄, and solvent was removed in vacuo.

General Procedure for 12a-b. 11a (1.36 mmol) was dissolved in CH₃CN (3.3 mL), then pyridine (10.8 mmol) was added. The solution was cooled to 0° C., and sulfonyl chloride (1.63 mmol) was added slowly by syringe. The solution was allowed to warm to room temperature. Reaction progress was monitored by TLC (5% MeOH in CHCl₃). When complete, the reaction was quenched by adding saturated NaHCO₃ solution. The product was extracted with EtOAc (3×5 mL), washed with 1 N HCl, and the combined organic extracts were dried over MgSO₄ and concentrated in vacuo. The product was purified by flash chromatography (2% MeOH in CHCl₃).

General Procedure for 12c-f. Sulfonyl chloride (4.9 mmol) was added dropwise to a solution of 11b-c (3.3 mmol) in CH₃CN (13 mL) at 0° C. Et₃N (3.63 mmol) was added dropwise, and the solution was stirred and allowed to warm to room temperature. Reaction progress was monitored by TLC (10% MeOH in CHCl₃). When complete (about 2 h), the reaction was quenched by adding saturated sodium bicarbonate solution. The product was extracted with EtOAc (3×10 mL), and the combined organic extracts were dried over MgSO₄ and concentrated in vacuo. Flash chromatography (2% MeOH in CHCl₃) afforded the product.

General Procedure for 13a-f. TMSBr (8.6 mmol) was added to a solution of 12a-f (0.277 mmol) in CH₂Cl₂ (2 mL), and the solution was stirred at room temperature. After 24 h, the reaction was quenched by adding MeOH (3×1.6 mL). The solution was concentrated in vacuo, and dissolved in saturated NaHCO₃ solution (10 mL). This solution was washed with Et₂O (5 mL), then acidified with 1 N HCl. The product was extracted with Et₂O (3×5 mL), and the combined organic extracts were dried over MgSO₄ and dried in vacuo.

2-(Pentylsulfonamido)benzylphosphonic acid 13a. ¹H NMR (DMSO-d₆) δ 9.73 (s, 1H), 7.38 (d, J=8.0 Hz, 1H), 7.25 (m, 2H), 7.13 (t, J=7.6 Hz, 1H), 3.14 (t, J=8.0 Hz, 2H), 3.10 (d, J=20.8 Hz, 2H), 1.71 (m, 2H), 1.32 (m, 4H), 0.85 (t, J=7.2 Hz, 3H); ¹³C NMR (DMSO-d₆) δ 136.2 (d, J=5.8 Hz), 131.6 (d, J=6.4 Hz), 127.6 (d, J=10.0 Hz), 127.1 (d, J=3.4 Hz), 125.0 (d, J=2.8 Hz), 123.7 (d, J=3.0 Hz), 52.3, 32.5 (d, J=130.3 Hz), 29.4, 22.7, 21.3, 13.3.

2-(Nonylsulfonamido)benzylphosphonic acid 13b. mp=104-106° C.; ¹H NMR (DMSO-d₆) δ 9.80 (s, 1H), 7.37 (d, J=8.0 Hz, 1H), 7.25 (m, 2H), 7.15 (t, J=7.6 Hz, 1H), 3.14 (t, J=7.6 Hz, 2H), 3.09 (d, J=21.2 Hz, 2H), 1.68 (m, 2H), 1.35 (m, 2H), 1.22 (m, 8H), 0.85 (t, J=6.8 Hz); ¹³C NMR (DMSO-d₆) δ 136.3 (d, J=5.7 Hz), 131.8 (d, J=6.5 Hz), 127.7 (d, J=8.7 Hz), 127.3 (d, J=3.3 Hz), 125.2 (d, J=2.6 Hz), 123.8 (d, J=3.0 Hz), 52.3, 32.6 (d, J=130.5 Hz), 31.2, 28.6, 28.5, 28.4, 27.4, 23.2, 22.0, 13.9.

3-(Pentylsulfonamido)benzylphosphonic acid 13c. mp=127-128° C.; ¹H NMR (MeOD) δ 7.27 (t, J=8.0 Hz, 1H), 7.22 (s, 1H), 7.11 (m, 2H), 3.11 (d, J=21.6 Hz, 2H), 3.09 (t, J=7.8 Hz, 2H), 1.78 (m, 2H), 1.38 (m, 4H), 0.91 (t, J=6.4 Hz, 3H); ¹³C NMR (MeOD) δ 139.5 (d, J=3.3 Hz), 136.0 (d, J=9.3 Hz), 130.3 (d, J=3.4 Hz), 126.8 (d, J=5.9 Hz), 122.5 (d, J=6.5 Hz), 119.3 (d, J=3.4 Hz), 51.9, 35.8 (d, J=134.2 Hz), 31.2, 24.2, 23.1, 14.0; HRMS (FAB) calcd for C₁₂H₂₁NO₅PS [M+H]⁺, 322.08781; found, 322.08830.

3-(Nonylsulfonamido)benzylphosphonic acid 13d. mp=149-150° C.; ¹H NMR (MeOD) δ 7.27 (t, J=8.0 Hz, 1H), 7.22 (s, 1H), 7.11 (m, 2H), 3.11 (d, J=21.6 Hz, 2H), 3.08 (t, J=7.6 Hz, 2H), 1.77 (m, 2H), 1.33 (m, 12H), 0.91 (t, J=6.4 Hz, 3H); ¹³C NMR (MeOD) δ 139.5 (d, J=3.0 Hz), 130.3 (d, J=3.0 Hz), 126.8 (d, J=6.1 Hz), 122.5, (d, J=6.3 Hz), 119.3 (d, J=3.5 Hz), 51.9, 35.8 (d, J=134.0 Hz), 32.9, 30.3, 30.2, 30.1, 29.1, 24.5, 23.6, 14.3; HRMS (FAB) calcd for C₁₆H₂₉NO₅PS [M+H]⁺, 378.15041; found, 378.14975.

4-(Pentylsulfonamido)benzylphosphonic acid 13e. mp=198-200° C.; ¹H NMR (MeOD) δ 7.29 (dd, J=8.4, 2.4 Hz, 2H), 7.20 (d, J=8.4 Hz, 2H), 3.10 (d, J=21.6 Hz, 2H), 3.05 (t, J=8.0 Hz, 2H), 1.78 (m, 2H), 1.34 (m, 4H), 0.90 (t, J=7.2 Hz, 3H); ¹³C NMR (MeOD) δ 137.9 (d, J=3.7 Hz), 131.8 (d, J=6.3 Hz), 130.6 (d, J=9.6 Hz), 121.4 (d, J=2.9 Hz), 51.8 35.1 (d, J=134.6 Hz), 31.2, 24.2, 23.1, 14.0; HRMS (FAB) calcd for C₁₂H₂₀NO₅PS [M]⁺, 321.07998; found, 321.07934.

4-(Nonylsulfonamido)benzylphosphonic acid 13f. mp=201-203° C.; ¹H NMR (MeOD) δ 7.29 (dd, J=8.8, 2.4 Hz, 2H), 7.20 (d, J=8.4 Hz, 2H), 3.09 (d, J=21.2, 2H), 3.05 (t, J=8.0 Hz, 2H), 1.77 (m, 2H), 1.29 (m, 12H), 0.91 (t, J=7.2 Hz, 3H); ¹³C NMR (MeOD) δ 137.9 (d, J=3.3 Hz), 131.8 (d, J=6.5 Hz), 130.6 (d, J=9.3 Hz), 121.4 (d, J=3.0 Hz), 51.8 35.1 (d, J=134.5 Hz), 32.9, 30.3, 30.2, 30.1, 29.1, 24.5, 14.4; HRMS (FAB) calcd for C₁₆H₂₉NO₅PS [M+H]⁺, 378.15041; found, 378.14945.

### Example 4

### Synthesis of Compounds 15a-15i

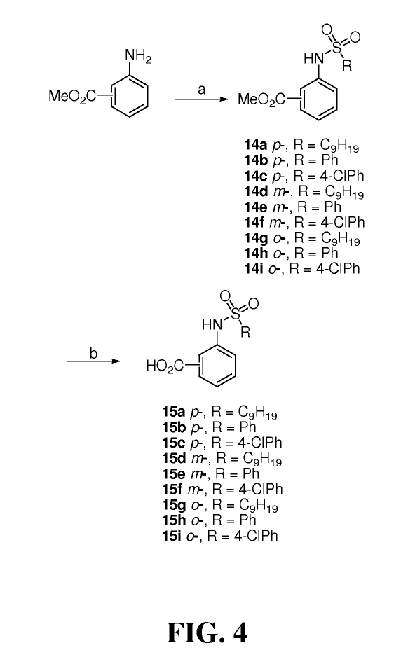

Synthesis of compounds 15a-15i was performed using Scheme 4, as illustrated in FIG. 4 herein.

Reaction conditions: (a) RSO₂Cl, pyridine, CH₂Cl₂, 0° C. to rt; (b) K⁺O⁻t-Bu, Et₂O, H₂O, 0° C. to room temperature.

Compounds 15a-i were synthesized by coupling the commercially available starting aniline with a variety of sulfonyl chlorides. The resulting sulfonamides 14a-i were then converted to the final products by hydrolysis with potassium t-butoxide and water in ether. Aromatic sulfonyl chlorides were used as well as the saturated C₉ chain in an attempt to mimic the CoA portion of the acyl-CoA substrate, as opposed to the alkyl chain.

General Procedure for 14a-i. To a stirring solution of the aniline starting material (3.3 mmol) in CH₂Cl₂ (12 mL) at 0° C. was added pyridine (7.5 equiv) was added. The sulfonyl chloride (1.2 equiv) was then added slowly via syringe. The solution was stirred and allowed to warm to room temperature. Reaction progress was monitored by TLC (20% EtOAc in hexanes). When complete, the reaction was poured into saturated NaHCO₃ solution (45 mL), extracted with CH₂Cl₂ (3×15 mL), and washed with 1 M HCl (50 mL). The combined organic phases were concentrated in vacuo, and recrystallization from EtOAc/hexanes afforded 14a-i.

General Procedure for 15a-i. To a stirring suspension of potassium t-butoxide (5.88 mmol) in Et₂O (15mL) cooled to 0° C., was added water (1.4 mmol) via syringe. The slurry was stirred for 5 min, and 14a-i (0.67 mmol) was added. The mixture was stirred at room temperature until starting material disappeared by TLC analysis (20% EtOAc in hexanes). Ice water was added until 2 clear layers formed. The aqueous layer was separated and acidified with 1 M HCl. The product was then extracted with Et₂O (3×20 mL) and evaporated in vacuo to afford 15a-i.

4-(Nonylsulfonamido)benzoic acid 15a. mp=193-194° C.; ¹H NMR (MeOD) δ 7.99 (d, J=8.0 Hz, 2H), 7.31 (d, J=8.4 Hz, 2H), 3.17 (t, J=8.0 Hz, 2H), 1.78 (m, 2H), 1.40 (m, 2H), 1.28 (m, 10H), 0.89 (t, J=7.2 Hz, 3H); ¹³C NMR (MeOD) δ 169.3, 144.3, 132.3, 126.7, 118.8, 52.4, 32.9, 30.2, 30.2, 30.0, 28.9, 24.4, 23.6, 14.3; HRMS (FAB) calcd for C₁₆H₂₅NO₄S [M]⁺, 327.15043; found, 327.14957.

4-(Phenylsulfonamido)benzoic acid 15b. mp=186-188° C.; ¹H NMR (DMSO-d₆) δ 12.72 (br s, 1H), 10.82 (br s, 1H), 7.80 (m, 4H), 7.61 (t, J=6.8 Hz, 1H), 7.56 (t, J=8.0 Hz, 2H), 7.20 (t, J=7.2 Hz, 2H); ¹³C NMR (DMSO-d₆) δ 166.6, 141.9, 142.0, 133.2, 130.7, 129.4, 126.6, 125.6, 118.2.; HRMS (FAB) calcd for C₁₃H₁₁NO₄S [M]⁺, 277.04088; found, 277.04077.

4-(4-Chlorophenylsulfonamido)benzoic acid 15c. mp=254-256° C.; ¹H NMR (DMSO-d₆) δ 12.76 (br s, 1H), 10.86 (br s, 1H), 7.81 (d, J=6.4 Hz, 4H), 7.65 (d, J=7.2 Hz, 2H), 7.18 (d, J=6.8 Hz, 2H); ¹³C NMR (DMSO-d₆) δ 166.6, 141.5, 138.1, 138.0, 130.7, 129.5, 128.5, 125.9, 118.4; HRMS (FAB) calcd for C₁₃H₁₁ClNO₄S [M+H]⁺, 312.00973; found, 312.00859.

3-(Nonylsulfonamido)benzoic acid 15d. mp=183-184° C.; ¹H NMR (DMSO-d₆) δ 13.03 (br s, 1H), 9.98 (s, 1H), 7.81 (s, 1H), 7.64 (m, 1H), 7.44 (m, 2H), 3.07 (t, J=7.6 Hz, 2H), 1.65 (m, 2H), 1.21 (m, 12H), 0.83 (t, J=7.2 Hz, 3H); ¹³C NMR (DMSO-d₆) δ 166.8, 138.7, 131.8, 129.5, 124.3, 123.2, 119.7, 50.5, 31.1, 28.5, 28.5, 28.3, 27.1, 22.9, 22.0, 13.8; HRMS (FAB) calcd for C₁₆H₂₆NO₄S [M+H]⁺, 328.15826; found, 328.15640.

3-(Phenylsulfonamido)benzoic acid 15e. mp=203-204° C.; ¹H NMR (DMSO-d₆) δ 13.02 (br s, 1H), 10.51 (br s, 1H), 7.75 (d, J=7.2 Hz, 2H), 7.68 (s, 1H), 7.56 (m, 4H), 7.34 (m, 2H); ¹³C NMR (DMSO-d₆) δ 166.6, 139.2, 137.9, 133.0, 131.7, 129.4, 129.3, 126.5, 124.8, 124.0, 120.5; HRMS (FAB) calcd for C₁₃H₁₁NO₄S [M]⁺, 277.04088; found, 277.04054.

3-(4-Chlorophenylsulfonamido)benzoic acid 15f. mp=242-243° C.; ¹H NMR (MeOD) δ 7.76 (d, J=8.8 Hz, 4H), 7.52 (d, J=8.4 Hz, 2H), 7.34 (m, 2H); ¹³C NMR (MeOD) δ 168.9, 140.2, 139.5, 139.0, 133.0, 130.3, 130.3, 129.8, 127.0, 126.4, 123.1; HRMS (FAB) calcd for C₁₃H₁₀ClNO₄S [M]⁺, 311.00191; found, 311.00152.

C67-2-(Nonylsulfonamido)benzoic acid 15g. mp=122-124° C.; ¹H NMR (MeOD) δ 8.11 (d, J=8.0 Hz, 1H), 7.73 (d, J=8.4 Hz, 1H), 7.59 (t, J=7.6 Hz, 1H), 7.16 (t, J=7.6 Hz, 1H), 3.18 (t, J=8.0 Hz, 2H), 1.71 (m, 2H), 1.24 (m, 12H), 0.88 (t, J=7.2 Hz, 3H); ¹³C NMR (MeOD) δ 171.3, 142.4, 135.7, 133.1, 123.8, 118.8, 117.0, 52.3, 32.9, 30.2, 30.1, 29.9, 28.8, 24.4, 23.6, 14.4; HRMS (FAB) calcd for C₁₆H₂₅NO₄S [M]⁺, 327.15043; found, 327.15044.

2-(Phenylsulfonamido)benzoic acid 15h. mp=213-215° C.; ¹H NMR (MeOD) δ 7.95 (d, J=8.0 Hz, 1H), 7.81 (d, J=8.0 Hz, 2H), 7.69 (d, J=8.4 Hz, 1H), 7.56 (t, J=7.6 Hz, 1H), 7.49 (m, 3H), 7.09 (t, J=7.6 Hz, 1H); ¹³C NMR (DMSO-d₆) δ 169.7, 139.7, 138.5, 134.4, 133.5, 131.5, 129.4, 126.8, 123.3, 118.4, 116.7; HRMS (FAB) calcd for C₁₃H₁₁NO₄S [M]⁺, 277.04088; found, 277.04124.

2-(4-Chlorophenylsulfonamido)benzoic acid 15i. mp=202-203° C.; ¹H NMR (DMSO-d₆) δ 13.98 (br s, 1H), 11.12 (br s, 1H), 7.90 (d, J=8.0 Hz, 1H), 7.81 (d, J=8.8 Hz, 2H), 7.63 (d, J=8.8 Hz, 2H), 7.54 (t, J=7.6 Hz, 1H), 7.48 (d, J=8.4 Hz, 1H), 7.14 (t, J=7.2 Hz, 1H); ¹³C NMR (MeOD) δ 171.1, 141.3, 140.6, 139.0, 135.4, 132.8, 130.3, 129.9, 124.7, 120.6, 118.3; HRMS (FAB) calcd for C₁₃H₁₀ClNO₄S [M]⁺, 311.00191; found, 311.00136.

### Example 5

### Synthesis of Compounds 17a-17f

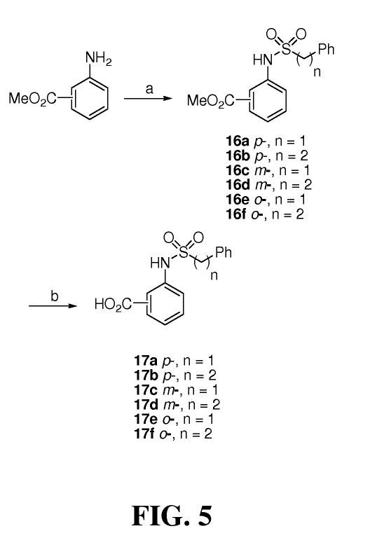

Synthesis of compounds 17a-17f was performed using Scheme 5, as illustrated in FIG. 5 herein.

Reaction conditions: (a) RSO₂Cl, pyridine, CH₂Cl₂, 0° C. to rt; (b) K⁺O⁻t-Bu, Et₂O, H₂O, 0° C. to room temperature.

Compounds 17a-f were designed to probe the effect of linkers of different length in the aryl sulfonamide portion of the molecule. These were produced in the same manner as cmpounds 15a-i, starting with the commercially available aniline and coupling to either benzylsulfonyl chloride or phenylethylsulfonyl chloride with pyridine in methylene chloride to yield sulfonamides 16a-f. The methyl esters were then converted to the carboxylic acids 17a-f with potassium t-butoxide and water in ether.

General Procedure for 16a-f. To a stirring solution of the aniline starting material (3.3 mmol) in CH₂Cl₂ (12 mL) at 0° C. was added pyridine (7.5 equiv). The sulfonyl chloride (1.2 equiv) was then added slowly via syringe. The solution was stirred and allowed to warm to room temperature. Reaction progress was monitored by TLC (20% EtOAc in hexanes). When complete, the reaction was poured into saturated NaHCO₃ (45 mL), extracted with CH₂Cl₂ (3×15 mL), and washed with 1 M HCl (50 mL). The combined organic phases were concentrated in vacuo, and the resulting solid was recrystallized from EtOAc/hexanes to afford 16a-f.

General Procedure for 17a-f. To a stirring suspension of potassium t-butoxide (5.88 mmol) in Et₂O (15 mL) cooled to 0° C., was added water (1.4 mmol) via syringe. The slurry was stirred for 5 min, and 16a-f (0.67 mmol) was added. The mixture was stirred at room temperature until starting material disappeared by TLC analysis (20% EtOAc in hexanes). Ice water was added until 2 clear layers formed. The aqueous layer was separated and acidified with 1 M HCl. The product was then extracted with Et₂O (3×20 mL) and evaporated in vacuo to afford 17a-f.

4-(Phenylmethylsulfonamido)benzoic acid 17a. mp=221-223° C.; ¹H NMR (DMSO-d₆) δ 12.72 (br s, 1H), 10.29 (s, 1H), 7.88 (d, J=8.4 Hz, 2H), 7.33 (m, 3H), 7.24 (m, 4H), 4.56 (s, 2H); ¹³C NMR (DMSO-d₆) δ 166.8, 142.7, 130.9, 130.8, 129.2, 128.3, 128.3, 124.8, 117.2, 57.1; HRMS (FAB) calcd for C₁₄H₁₄NO₄S [M+H]⁺, 292.06435; found, 292.06397.

4-(2-Phenylethylsulfonamido)benzoic acid 17b. mp=222-223° C.; ¹H NMR (DMSO-d₆) δ 12.74 (br s, 1H), 10.38 (s, 1H), 7.90 (d, J=8.0 Hz, 2H), 7.26 (m, 2H), 7.23 (m, 2H), 7.18 (m, 3H), 3.48 (t, J=6.4, 2H), 2.98 (t, J=6.4 Hz, 2H); ¹³C NMR (DMSO-d₆) δ 166.8, 142.4, 137.8, 130.8, 128.4, 128.3, 126.5, 125.2, 117.8, 51.9, 29.0; HRMS (FAB) calcd for C₁₅H₁₆NO₄S [M+H]⁺, 306.08000; found, 306.07892.

3-(Phenylmethylsulfonamido)benzoic acid 17c. mp=205-206° C.; ¹H NMR (DMSO-d₆) δ 13.02 (br s, 1H), 10.06 (s, 1H), 7.79 (s, 1H), 7.64 (d, J=7.2 Hz, 1H), 7.42 (m, 2H), 7.33 (m, 3H), 7.25 (m, 2H), 4.48 (s, 2H); ¹³C NMR (DMSO-d₆) δ 166.9, 138.7, 131.8, 130.9, 129.4, 129.3, 128.3, 128.2, 124.1, 122.9, 119.4, 57.0; HRMS (FAB) calcd for C₁₄H₁₄NO₄S [M+H]⁺, 292.06435; found, 292.06448.

3-(2-Phenylethylsulfonamido)benzoic acid 17d. mp=199-200° C.; ¹H NMR (DMSO-d₆) δ 13.06 (s, 1H), 10.11 (s, 1H), 7.85 (s, 1H), 7.67 (d, J=6.8 Hz, 1H), 7.48 (m, 2H), 7.24 (m, 2H), 7.17 (m, 3H), 3.38 (t, J=8.0 Hz, 2H), 2.99 (t, J=8.0 Hz, 2H); ¹³C NMR (DMSO-d₆) δ 166.8, 138.5, 137.9, 131.9, 129.6, 128.4, 128.3, 126.5, 124.6, 123.8, 120.2, 51.7, 29.0; HRMS (FAB) calcd for C₁₅H₁₆NO₄S [M+H]⁺, 306.08000; found, 306.08051.

2-(Phenylmethylsulfonamido)benzoic acid 17e. mp=216-219° C.; ¹H NMR (DMSO-d₆) δ 13.86 (br s, 1H), 10.68 (s, 1H), 7.99 (d, J=7.6 Hz, 1H), 7.58 (m, 2H), 7.32 (m, 3H), 7.19 (m, 3H), 4.69 (s, 2H); ¹³C NMR (DMSO-d₆) δ 169.6, 140.7, 134.6, 131.5, 130.7, 128.8, 128.4, 128.3, 122.4, 117.2, 115.4, 57.2; HRMS (FAB) calcd for C₁₄H₁₃NO₄S [M]⁺, 291.05653; found, 291.05655.

2-(2-Phenylethylsulfonamido)benzoic acid 17f. mp=157-159° C.; ¹H NMR (DMSO-d₆) δ 13.90 (br s, 1H), 10.74 (br s, 1H), 7.98 (d, J=8.0 Hz, 1H), 7.61 (d, J=4.4 Hz, 2H), 7.20 (m, 2H), 7.16 (m, 4H), 3.61 (t, J=8.0 Hz, 2H), 2.98 (t, J=8.0 Hz, 2H); ¹³C NMR (DMSO-d₆) δ 169.7, 140.3, 137.5, 134.6, 131.6, 128.3, 128.2, 126.5, 122.6, 117.7, 115.9, 52.0, 28.9; HRMS (FAB) calcd for C₁₅H₁₆NO₄S [M+H]⁺, 306.08000; found, 306.07886.

### Example 6

### Synthesis of Compounds 21a-21c

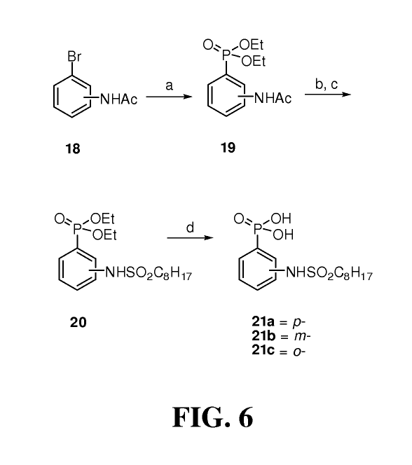

Synthesis of compounds 21a-21c was performed using Scheme 6, as illustrated in FIG. 6 herein.

Reaction conditions: (a) diethyl phosphite, Et₃N, Pd(PPh₃)₄, EtOH, reflux; (b) H₂SO₄, EtOH, reflux; (c) C₈H₁₇SO₂Cl, Et₃N, CH₂Cl₂, 0° C. to room temperature; (d) TMSBr, CH₂Cl₂, room temperature.

The synthesis of aryl phosphonic acids 21a-c is shown in Scheme 6. Aryl bromide 18 underwent palladium-catalyzed aryl halide coupling with diethyl phosphite to install the phosphonate functionality. (Gooβen, L. J., et. al.; Dezfuli, M. K. Practical Protocol for the Palladium-Catalyzed Synthesis of Arylphosphonates from Bromoarenes and Diethyl Phosphite, Synlett 2005, 3, 445). The aniline was then deprotected by refluxing in acidic ethanol, and the free amine was coupled with commercially-available octanesulfonyl chloride to produce 20. The final compound was then obtained by deprotecting the diethyl phosphonate with TMSBr.

General Procedure for 19a-c. The starting bromide 18 (1.96 mmol) was added to a round-bottomed flask containing diethyl phosphite (2.35 mmol), tetrakis(triphenylphosphine)palladium (0) (0.04 mmol), Et₃N (2.94 mmol), and EtOH (8 mL), and the solution was heated to reflux overnight (16 h). The solution was then diluted with 30 mL EtOAc, washed with 50 mL saturated NaHCO₃ solution, 50 mL H₂O, dried over MgSO₄, and concentrated in vacuo. The product was then purified by flash chromatography (EtOAc).

4-(Octylsulfonamido)phenylphosphonic acid 21a. mp=185-187° C.; ¹H NMR (MeOD) δ 7.75 (dd, J=12.8, 8.0 Hz, 2H), 7.33 (dd, J=8.0, 3.2 Hz, 2H), 3.15 (t, J=8.0 Hz, 2H), 1.78 (m, 2H), 1.39 (m, 2H), 1.28 (m, 8H), 0.90 (t, J=7.2 Hz, 3H); ¹³C NMR (MeOD) δ 143.0 (d, J=3.6 Hz), 133.4 (d, J=11.0 Hz), 127.7 (d, J=190 Hz), 119.1 (d, J=15.2 Hz), 52.4, 32.8, 30.0, 29.9, 29.0, 24.5, 23.6, 14.3; HRMS (FAB) calcd for C₁₄H₂₅NO₅PS [M+H]⁺, 350.11911; found, 350.11869.

3-(Octylsulfonamido)phenylphosphonic acid 21b. mp=112-114° C.; ¹H NMR (MeOD) δ 7.72 (d, J=14.8 Hz, 1H), 7.55 (m, 1H), 7.44 (m, 2H), 3.12 (t, J=8.0 Hz, 2H), 1.78 (m, 2H), 1.39 (m, 2H), 1.27 (m, 8H), 0.90 (t, J=7.2 Hz, 3H); ¹³C NMR (MeOD) δ 139.6 (d, J=18.2 Hz), 134.5 (d, J=184 Hz), 130.5 (d, J=16.1 Hz), 127.3 (d, J=9.5 Hz), 123.8 (d, J=3.0 Hz), 122.8 (d, J=11.6 Hz), 52.2, 32.7, 30.0, 29.9, 29.0, 24.4, 23.5, 14.3; HRMS (FAB) calcd for C₁₄H₂₅NO₅PS [M+H]⁺, 350.11911; found, 350.11879.

2-(Octylsulfonamido)phenylphosphonic acid 21c. mp=92-94° C.; ¹H NMR (MeOD) δ 7.70 (m, 2H), 7.54 (t, J=8.4 Hz, 1H), 7.21 (t, J=7.5 Hz, 1H), 3.18 (t, J=7.8 Hz, 2H), 1.77 (m, 2H), 1.23 (m, 10H), 0.89 (t, J=7.5 Hz, 3H); ¹³C NMR (MeOD) δ 141.8 (d, J=7.0 Hz), 134.3 (d, J=2.7 Hz), 134.1 (d, J=6.8 Hz), 120.4 (d, J=178 Hz), 119.6 (d, J=10.8 Hz), 52.6, 32.8, 29.9, 29.9, 29.0, 24.3, 23.5, 14.3; HRMS (FAB) calcd for C₁₄H₂₅NO₅PS [M+H]⁺, 350.11911; found, 350.11826.

### Example 7

### Synthesis of Compounds 24a-24f

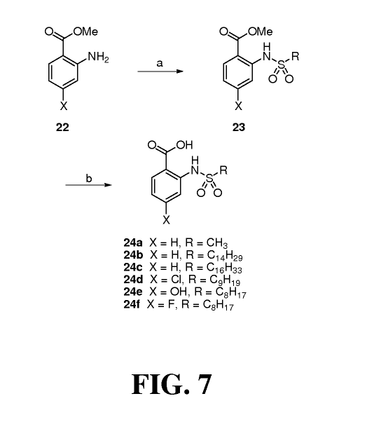

Synthesis of compounds 24a-24f was performed using Scheme 7, as illustrated in FIG. 7 herein.

Reaction conditions: (a) RSO₂Cl, pyridine, CH₂Cl₂, 0° C. to room temperature; (b) K⁺O⁻t-Bu, Et₂O, H₂O, 0° C. to room temperature.

Compounds 24a-c, based on 15g, were designed as probes to examine the effect of installing different length alkylsulfonamides on the ortho-substituted analogs. It was believed that the compound with the saturated C₁₆-chain (24c) would exhibit significantly greater inhibitory activity than 15g, as the enzyme demonstrates a marked preference for palmitoyl-CoA over other long-chain acyl-CoAs.¹³ Compounds 24d-f were designed to examine the role of an electronegative group at the 4-position of the benzene ring, which could possibly mimic the electron density of the secondary alcohol on glycerol-3-phosphate. All of these compounds (24a-f) were produced with the same reaction sequence used to produce 15a-f and 17a-f.

General Procedure for 23a-f. To a stirring solution of the aniline starting material (3.3 mmol) in CH₂Cl₂ (12 mL) at 0° C. was added pyridine (7.5 equiv). The sulfonyl chloride (1.2 equiv) was then added slowly via syringe. The solution was stirred and allowed to warm to room temperature. Reaction progress was monitored by TLC (20% EtOAc in hexanes). When complete, the reaction was poured into saturated NaHCO₃ solution (45 mL), extracted with CH₂Cl₂ (3×15 mL), and washed with 1 M HCl (50 mL). The combined organic phases were concentrated in vacuo, and separated by flash chromatography (20% EtOAc in hexanes) to afford 23a-f.

General Procedure for 24a-f. To a stirring suspension of potassium t-butoxide (5.88 mmol) in Et₂O (15 mL) cooled to 0° C. was added water (1.4 mmol) via syringe. The slurry was stirred for 5 min, and 23a-f (0.67 mmol) was added. The mixture was stirred at room temperature until starting material disappeared by TLC analysis (20% EtOAc in hexanes). Ice water was added until two clear layers formed. The aqueous layer was separated and acidified with 1 M HCl, and the product was extracted with Et₂O (3×20 mL) and evaporated in vacuo. If necessary, the product was then recrystallized (EtOAc/hexanes) to afford pure 24a-f.

2-(Methylsulfonamido)benzoic acid 24a. mp=187-189° C.; ¹H NMR (MeOD) δ 8.11 (d, J=8.0 Hz, 1H), 7.70 (d, J=8.0 Hz, 1H), 7.60 (t, J=7.2 Hz, 1H), 7.17 (t, J=7.6 Hz, 1H), 3.08 (s, 3H); ¹³C NMR (MeOD) δ 171.2, 142.2, 135.7, 133.0, 123.9, 119.2, 117.3, 39.9; HRMS (FAB) calcd for C₈H₉NO₄S [M]⁺, 215.02523; found, 215.02576.

2-(Tetradecylsulfonamido)benzoic acid 24b. mp=120-122° C.; ¹H NMR (MeOD) δ 8.12 (d, J=8.0 Hz, 1H), 7.74 (d, J=8.4 Hz, 1H), 7.59 (t, J=7.6 Hz, 1H), 7.17 (t, J=7.6 Hz, 1H), 3.19 (t, J=8.0 Hz, 2H), 1.70 (m, 2H), 1.29 (m, 22H), 0.91 (t, J=6.8 Hz, 3H); ¹³C NMR (MeOD) δ 171.4, 142.5, 135.7, 133.1, 123.8, 118.8, 117.0, 52.2, 33.0, 30.7, 30.7, 30.7, 30.6, 30.5, 30.4, 30.2, 29.9, 28.8, 24.4, 23.7, 14.4.

2-(Hexadecylsulfonamido)benzoic acid 24c. mp=126-128° C.; ¹H NMR (MeOD) δ 8.12 (d, J=7.6 Hz, 1H), 7.74 (d, J=8.4 Hz, 1H), 7.59 (t, J=8.0 Hz, 1H), 7.17 (t, J=7.6 Hz, 1H), 3.19 (t, J=7.6 Hz, 2H), 1.73 (m, 2H), 1.23 (m, 26H), 0.91 (t, J=7.2 Hz, 3H); ¹³C NMR (MeOD) δ 169.8, 140.7, 134.6, 131.6, 122.4, 117.3, 115.6, 50.9, 31.2, 29.0, 29.0, 29.0, 29.0, 29.0, 28.9, 28.8, 28.7, 28.5, 28.2, 27.0, 22.8, 22.0, 13.8; HRMS (FAB) calcd for C₂₃H₄₀NO₄S [M+H]⁺, 426.26781; found, 426.26825.

5-Chloro-2-(nonylsulfonamido)benzoic acid 24d. mp=101-103° C.; ¹H NMR (MeOD) δ 8.05 (d, J=2.8 Hz, 1H), 7.74 (d, J=9.2 Hz, 1H), 7.59 (dd, J=9.2, 2.8 Hz, 1H), 3.21 (t, J=8.0 Hz, 2H), 1.72 (m, 2H), 1.23 (m, 12H), 0.90 (t, J=7.2 Hz, 3H); ¹³C NMR (MeOD) δ 170.1, 141.2, 135.5, 132.4, 128.9, 120.6, 118.0, 52.5, 32.9, 30.2, 30.1, 29.9, 28.8, 24.4, 23.6, 14.4; HRMS (FAB) calcd for C₁₆H₂₄C1NO₄S [M]⁺, 361.11146; found, 361.11063.

5-Hydroxy-2-(octylsulfonamido)benzoic acid 24e. mp=142-144° C.; ¹H NMR (MeOD) δ 7.56 (d, J=8.8 Hz, 1H), 7.51 (d, J=2.8 Hz, 1H), 7.03 (dd, J=8.8, 2.8 Hz, 1H), 3.07 (t, J=8.0 Hz, 2H), 1.68 (m, 2H), 1.23 (m, 10H), 0.89 (t, J=7.2 Hz, 3H); ¹³C NMR (MeOD) δ 171.0, 154.6, 134.1, 122.8, 121.9, 119.2, 118.5, 53.1, 32.8, 29.9, 29.8, 28.8, 24.3, 23.6, 14.4.

5-Fluoro-2-(octylsulfonamido)benzoic acid 24f. mp=141-143° C.; ¹H NMR (MeOD) δ 7.77 (m, 2H), 7.38 (m, 1H), 3.17 (t, J=8.0 Hz, 2H), 1.71 (m, 2H), 1.23 (m, 10H), 0.88 (t, J=7.2 Hz, 3H); ¹³C NMR (MeOD) δ 170.2 (d, J=1.8 Hz), 159.2 (d, J=241 Hz), 138.6 (d, J=2.7 Hz), 122.7 (d, J=22.7 Hz), 121.5 (d, J=7.6 Hz), 119.0 (d, J=6.9 Hz), 118.8 (d, J=24.1 Hz), 52.4, 32.8, 29.9, 29.9, 28.8, 24.4, 23.6, 14.3; HRMS (FAB) calcd for C₁₅H₂₂FNO₄S [M]⁺, 331.12536; found, 331.12445.

### Example 8

### Synthesis of Compounds 4a-t and 7a-t

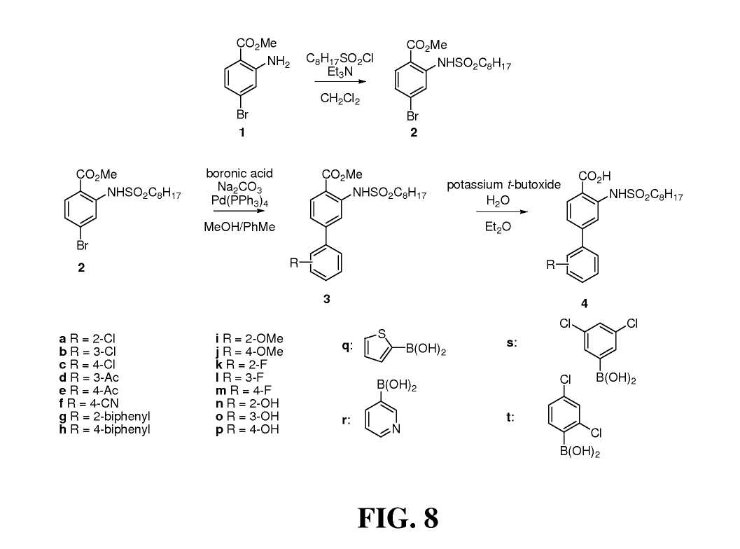

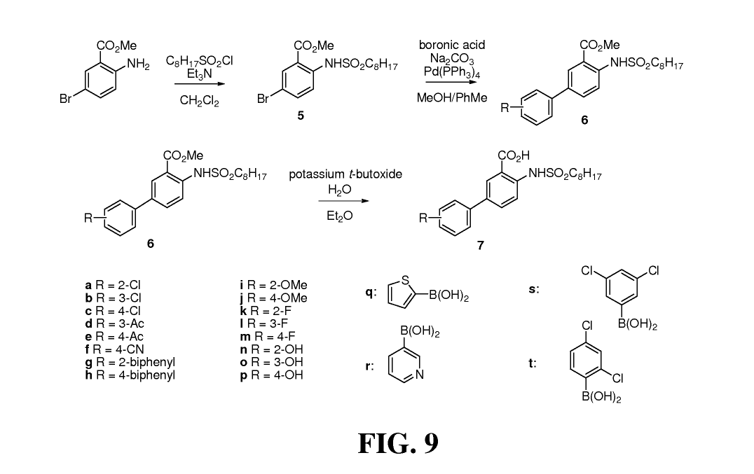

Synthesis of compounds 4a-t and 7-a-t was performed using Schemes illustrated in FIGS. 8 and 9, respectively, herein.

General Suzuki Reaction Experimental—0.247 mmol aryl bromide was placed into a vial flushed with argon, and a solution of 10 mg Pd(PPh₃)₄ in 0.40 mL toluene was added, followed by 0.25 mL 2M Na₂CO₃ solution. The solution was stirred at room temperature for 5 min, and then a solution of the boronic acid (1.25 equiv) in 0.40 mL MeOH was added. The vial was capped and heated to 90° C. for 24 h. The reaction was then cooled to room temperature and diluted with CH₂Cl₂, the organic phase was separated from the aqueous phase, and the organic phase was concentrated in vacuo. The crude product was purified by column chromatography (EtOAc/hexanes) to yield the desired bis-aryl product.

General Procedure for 4a-t and 7a-t. To a stifling suspension of potassium t-butoxide (2.00 mmol) in Et₂O (8 mL) cooled to 0° C., was added water (0.4 mmol) via syringe. The slurry was stirred for 5 min, and 3a-t or 6a-t (0.2 mmol) was added. The mixture was stirred at room temperature until starting material disappeared by TLC analysis (20% EtOAc in hexanes). Ice water was added until 2 clear layers formed. The aqueous layer was separated and acidified with 1 M HCl. The product was then extracted with Et₂O (3×20 mL) and evaporated in vacuo to afford 4a-t and 7a-t. If further purification was necessary, the product was purified by flash chromatograpy (1:1:8 AcOH: EtOAc: hexanes).

(4a) ¹H NMR (DMSO-d₆) δ 8.00 (d, J=8.0 Hz, 1H), 7.57 (m, 1H), 7.48 (s, 1H), 7.41 (m, 3H), 6.96 (d, J=8.0 Hz, 1H), 3.08 (t, J=8.0 Hz, 2H), 1.60 (m, 2H), 1.18 (m, 8H), 0.80 (t, J=7.2 Hz, 3H).

(4b) ¹H NMR (MeOD) δ 8.21 (d, J=8.4 Hz, 1H), 7.97 (s, 1H), 7.68 (s, 1H), 7.62 (d, J=8.4 Hz, 1H), 7.49 (m, 2H), 3.25 (t, J=8.4 Hz, 2H), 1.76 (m, 2H), 1.38 (m, 2H), 1.23 (m, 8H), 0.87 (t, J=7.2 Hz, 3H).

(4c) ¹H NMR (MeOD) δ 8.15 (d, J=8.0 Hz, 1H), 7.89 (d, J=1.6 Hz, 1H), 7.65 (d, J=6.8 Hz, 2H), 7.49 (d, J=6.8 Hz, 2H), 7.35 (dd, J=8.0, 2.0 Hz, 1H), 3.14 (t, J=8.0 Hz, 2H), 1.74 (m, 2H), 1.35 (m, 2H), 1.22 (m, 8H), 0.86 (t, J=6.8 Hz, 3H).

(4d) ¹H NMR (MeOD) δ 8.26 (m, 1H), 8.23 (d, J=8.4 Hz, 1H), 8.07 (m, 1H), 8.01 (d, J=1.5 Hz, 1H), 7.92 (m, 1H), 7.66 (t, J=7.8 Hz, 1H), 7.49 (dd, J=8.4, 1.8 Hz, 1H), 3.23 (t, J=7.8 Hz, 2H), 2.69 (s, 3H), 1.77 (m, 2H), 1.40 (m, 2H), 1.22 (m, 8H), 0.89 (t, J=7.2 Hz, 3H).

(4e) ¹H NMR (DMSO-d₆) δ 8.08 (m, 3H), 7.82 (m, 3H), 7.46 (dd, J=7.6, 1.6 Hz, 1H), 3.27 (t, J=7.6 Hz, 2H), 2.60 (s, 3H), 1.60 (m, 2H), 1.30 (m, 2H), 1.16 (m, 8H), 0.79 (t, J=6.8 Hz, 3H).

(4f) ¹H NMR (MeOD) δ 8.23 (d, J=8.4 Hz, 1H), 8.03 (d, J=1.6 Hz, 1H), 7.87 (m, 4H), 7.50 (dd, J=8.4, 1.6 Hz, 1H), 3.24 (t, J=8.0 Hz, 2H), 1.76 (m, 2H), 1.38 (m, 2H), 1.23 (m, 8H), 0.87 (t, J=7.2 Hz, 3H).

(4g) ¹H NMR (MeOD) δ 8.06 (d, J=8.4 Hz, 1H), 7.45 (m, 5H), 7.23 (m, 5H), 7.12 (dd, J=8.0, 1.2 Hz, 1H), 2.61 (t, J=8.0 Hz, 2H), 1.56 (m, 2H), 1.26 (m, 10H), 0.91 (t, J=7.2 Hz, 3H).

(4h) ¹H NMR (DMSO-d₆) δ 8.08 (d, J=8.4 Hz, 1H), 7.81 (m, 7H), 7.49 (m, 3H), 7.39 (t, J=7.2 Hz, 1H), 3.31 (t, J=8.0 Hz, 2H), 1.64 (m, 2H), 1.31 (m, 2H), 1.18 (m, 8H), 0.79 (t, J=6.8 Hz, 3H).

(4i) ¹H NMR (MeOD) δ 8.12 (d, J=8.4 Hz, 1H), 7.93 (s, 1H), 7.40 (m, 2H), 7.28 (d, J=8.4 Hz, 1H), 7.11 (m, 2H), 3.85 (s, 3H), 3.25 (t, J=7.8 Hz, 2H), 1.71 (m, 2H), 1.22 (m, 10H), 0.87 (t, J=6.9 Hz, 3H).

(4j) ¹H NMR (MeOD) δ 8.12 (d, J=8.4 Hz, 1H), 7.94 (d, J=1.6 Hz, 1H), 7.60 (d, J=6.8 Hz, 2H), 7.37 (dd, J=8.4, 1.6 Hz, 1H), 7.02 (d, J=6.8 Hz, 2H), 3.84 (s, 3H), 3.20 (t, J=7.6 Hz, 2H), 1.73 (m, 2H), 1.36 (m, 2H), 1.24 (m, 8H), 0.86 (t, J=7.2 Hz, 3H).

(4k) ¹H NMR (MeOD) δ 8.17 (m, 1H), 7.93 (m, 1H), 7.52 (m, 1H), 7.44 (m, 1H), 7.32 (m, 2H), 7.22 (m, 1H), 3.22 (t, J=8.0 Hz, 2H), 1.75 (m, 2H), 1.34 (2H), 1.22 (m, 8H), 0.83 (t, J=6.8 Hz, 3H).

(41) ¹H NMR (MeOD) δ 8.19 (d, J=8.4 Hz, 1H), 7.97 (s, 1H), 7.50 (m, 2H), 7.43 (t, J=8.0 Hz, 2H), 7.16 (m, 1H), 3.22 (t, J=7.6 Hz, 2H), 1.76 (m, 2H), 1.37 (m, 2H), 1.23 (m, 8H), 0.87 (t, J=7.2 Hz, 3H).

(4m) ¹H NMR (MeOD) δ 8.17 (d, J=8.0 Hz, 1H), 7.94 (d, J=1.6 Hz, 1H), 7.71 (m, 2H), 7.40 (dd, J=8.4, 2.0 Hz, 1H), 7.22 (t, J=8.8 Hz, 2H), 3.21 (t, J=7.6 Hz, 2H), 1.75 (m, 2H), 1.37 (m, 2H), 1.24 (m, 8H), 0.87 (t, J=7.2 Hz, 3H).

(4n) ¹H NMR (MeOD) δ 8.12 (m, 1H), 8.01 (m, 1H), 7.34 (m, 2H), 7.22 (m, 1H), 6.93 (m, 2H), 3.26 (t, J=8.0 Hz, 2H), 1.73 (m, 2H), 1.35 (m, 2H), 1.18 (m, 8H), 0.85 (t, J=7.2 Hz, 3H).

(4o) ¹H NMR (MeOD) δ 8.15 (d, J=8.4 Hz, 1H), 7.95 (s, 1H), 7.38 (d, J=8.4 Hz, 1H), 7.29 (t, J=7.6 Hz, 1H), 7.11 (m, 2H), 6.85 (m, 1H), 3.20 (t, J=8.0 Hz, 2H), 1.74 (m, 2H), 1.35 (m, 2H), 1.24 (m, 8H), 0.85 (t, J=7.2 Hz, 3H).

(4p) 8.10 (d, J=8.0 Hz, 1H), 7.92 (s, 1H), 7.53 (m, 2H), 7.36 (m, 1H), 6.89 (m, 2H), 3.19 (t, J=7.6 Hz, 2H), 1.72 (m, 2H), 1.34 (m, 2H), 1.19 (m, 8H), 0.84 (t, J=6.8 Hz, 3H).

(4q) ¹H NMR (MeOD) δ 8.10 (t, J=8.4 Hz, 1H), 8.01 (d, J=8.4 Hz, 1H), 7.53 (m, 2H), 7.43 (m, 1H), 7.18 (m, 1H), 3.22 (t, J=8.0 Hz, 2H), 1.74 (m, 2H), 1.19 (m, 8H), 0.82 (t, J=7.2 Hz, 3H).

(4r) ¹H NMR (MeOD) δ 8.84 (s, 1H), 8.57 (s, 1H), 8.21 (d, J=8.4 Hz, 1H), 8.13 (d, J=7.6 Hz, 1H), 7.92 (d, J=2.0 Hz, 1H), 7.56 (m, 1H), 7.40 (dd, J=8.0, 2.0 Hz, 1H), 3.15 (t, J=8.0 Hz, 2H), 1.77 (m, 2H), 1.36 (m, 2H), 1.22 (m, 8H), 0.86 (t, J=6.8 Hz, 3H).

(4s) ¹H NMR (MeOD) δ 8.15 (d, J=8.4 Hz, 1H), 7.88 (s, 1H), 7.52 (s, 2H), 7.42 (s, 1H), 7.33 (d, J=8.0 Hz, 1H), 3.22 (t, J=7.6 Hz, 2H), 1.72 (m, 2H), 1.35 (m, 2H), 1.23 (m, 8H), 0.84 (t, J=7.2 Hz, 3H).

(4t) ¹H NMR (MeOD) δ 8.18 (d, J=8.0 Hz, 1H), 7.79 (s, 1H), 7.60 (d, J=2.0 Hz, 1H), 7.43 (m, 2H), 7.19 (dd, J=8.0, 1.6 Hz, 1H), 3.23 (t, J=7.6 Hz, 2H), 1.74 (m, 2H), 1.36 (m, 2H), 1.21 (m, 8H), 0.87 (t, J=7.2 Hz, 3H).

(7a) ¹H NMR (MeOD) δ 8.17 (d, J=2.4 Hz, 1H), 7.76 (d, J=8.4 Hz, 1H), 7.60 (dd, J=8.4, 2.4 Hz, 1H), 7.49 (d, J=8.0 Hz, 1H), 7.37 (m, 3H), 3.21 (t, J=8.0 Hz, 2H), 1.76 (m, 2H), 1.20 (m, 10H), 0.86 (t, J=7.2 Hz, 3H).

(7b) ¹H NMR (MeOD) δ 8.29 (s, 1H), 7.81 (m, 2H), 7.57 (m, 1H), 7.52 (d, J=7.6 Hz, 1H), 7.42 (t, J=8.0 Hz, 1H), 7.34 (d, J=8.0 Hz, 1H), 3.22 (t, J=8.0 Hz, 2H), 1.72 (m, 2H), 1.23 (m, 10H), 0.83 (t, J=7.2 Hz, 3H).

(7c) ¹H NMR (MeOD) δ 8.32 (d, J=2.0 Hz, 1H), 7.81 (m, 2H), 7.58 (d, J=8.8 Hz, 2H), 7.43 (d, J=8.4 Hz, 2H), 3.22 (t, J=8.0 Hz, 2H), 1.73 (m, 2H), 1.35 (m, 2H), 1.20 (m, 8H), 0.84 (t, J=7.2 Hz, 3H).

(7d) ¹H NMR (MeOD) δ 8.37 (s, 1H), 8.19 (s, 1H), 7.97 (d, J=7.8 Hz, 1H), 7.81 (m, 3H), 7.57 (t, J=7.5 Hz, 1H), 3.21 (t, J=7.8 Hz, 2H), 2.66 (s, 3H), 1.74 (m, 2H), 1.20 (m, 10H), 0.83 (t, J=7.2 Hz, 3H).

(7e) ¹H NMR (MeOD) δ 8.42 (d, J=2.4 Hz, 1H), 8.12 (d, J=8.4 Hz, 2H), 7.99 (d, J=8.4 Hz, 1H), 7.89 (d, J=8.7 Hz, 1H), 7.81 (d, J=8.4 Hz, 2H), 3.29 (t, J=8.0 Hz, 2H), 2.66 (s, 3H), 1.77 (m, 2H), 1.37 (m, 2H), 1.24 (m, 8H), 0.89 (t, J=7.2 Hz, 3H).

(7f) ¹H NMR (MeOD) δ 8.40 (s, 1H), 7.89 (m, 1H), 7.80 (m, 5H), 3.23 (t, J=8.0 Hz, 2H), 1.73 (m, 2H), 1.36 (m, 2H), 1.21 (m, 8H), 0.84 (t, J=6.8 Hz, 3H).

(7g) ¹H NMR (MeOD) δ 7.89 (d, J=2.0 Hz, 1H), 7.51 (d, J=8.8 Hz, 1H), 7.38 (m, 4H), 7.19 (m, 4H), 7.09 (m, 2H), 3.10 (t, J=8.0 Hz, 2H), 1.65 (m, 2H), 1.21 (m, 10H), 0.86 (t, J=6.8 Hz, 3H).

(7h) ¹H NMR (DMSO-d₆) δ 8.31 (d, J=2.4 Hz, 1H), 8.01 (dd, J=8.8, 2.4 Hz, 1H), 7.74 (m, 7H), 7.48 (t, J=8.0 Hz, 2H), 7.38 (t, J=8.0 Hz, 1H), 3.33 (t, J=7.6 Hz, 2H), 1.63 (m, 2H), 1.31 (m, 2H), 1.20 (m, 8H), 0.81 (t, J=6.8 Hz, 3H).

(7i) ¹H NMR (MeOD) δ 8.24 (s, 1H), 7.71 (m, 2H), 7.28 (m, 2H), 6.98 (m, 2H), 3.77 (s, 3H), 3.17 (t, J=8.0 Hz, 2H), 1.70 (m, 2H), 1.19 (m, 10H), 0.83 (t, J=7.2 Hz, 3H).

(7j) ¹H NMR (MeOD) δ 8.26 (s, 1H), 7.71 (m, 2H), 7.46 (d, J=8.8 Hz, 2H), 6.93 (d, J=8.8 Hz, 2H), 3.79 (s, 3H), 3.15 (t, J=8.0 Hz, 2H), 1.70 (m, 2H), 1.20 (m, 10H), 0.81 (t, J=7.2 Hz, 3H).

(7k) ¹H NMR (MeOD) δ 8.27 (s, 1H), 7.78 (d, J=8.4 Hz, 1H), 7.70 (d, J=8.4 Hz, 1H), 7.42 (t, J=8.0 Hz, 1H), 7.32 (m, 1H), 7.19 (m, 2H), 3.19 (t, J=7.6 Hz, 2H), 1.71 (m, 2H), 1.32 (m, 2H), 1.16 (m, 8H), 0.81 (t, J=7.2 Hz, 3H).

(71) ¹H NMR (MeOD) δ 8.31 (t, J=1.2 Hz, 1H), 7.79 (s, 2H), 7.41 (m, 2H), 7.30 (dd, J=8.8, 1.6 Hz, 1H), 7.05 (dt, J=8.8, 1.6 Hz, 1H), 3.20 (t, J=8.0 Hz, 2H), 1.71 (m, 2H), 1.22 (m, 2H), 1.16 (m, 8H), 0.81 (t, J=6.8 Hz, 3H).

(7m) ¹H NMR (MeOD) δ 8.28 (d, J=2.0 Hz, 1H), 7.74 (m, 2H), 7.57 (dd, J=8.8, 4.8 Hz, 2H), 7.12 (t, J=8.4 Hz, 2H), 3.17 (t, J=8.0 Hz, 2H), 1.70 (m, 2H), 1.31 (m, 2H), 1.18 (m, 8H), 0.81 (t, J=6.8 Hz, 3H).

(7n) ¹H NMR (MeOD) δ 8.33 (s, 1H), 7.75 (dd, J=8.4, 2.4 Hz, 1H), 7.69 (d, J=8.4 Hz, 1H), 7.28 (dd, J=8.0, 1.6 Hz, 1H), 7.15 (dt, J=7.6, 1.6 Hz, 1H), 6.90 (m, 2H), 3.17 (t, J=8.0 Hz, 2H), 1.75 (m, 2H), 1.23 (m, 10H), 0.86 (t, J=7.2 Hz, 3H).

(7o) ¹H NMR (MeOD) δ 8.31 (s, 1H), 7.77 (s, 2H), 7.24 (t, J=8.0 Hz, 1H), 7.04 (m, 2H), 6.79 (d, J=8.0 Hz, 1H), 3.19 (t, J=8.0 Hz, 2H), 1.72 (m, 2H), 1.33 (m, 2H), 1.21 (m, 8H), 0.82 (t, J=6.8 Hz, 3H).

(7p) ¹H NMR (MeOD) δ 8.32 (s, 1H), 7.64 (m, 2H), 7.46 (m, 2H), 6.86 (m, 2H), 3.13 (t, J=8.0 Hz, 2H), 1.73 (m, 2H), 1.34 (m, 2H), 1.20 (m, 8H), 0.83 (t, J=6.8 Hz, 3H).

(7q) ¹H NMR (MeOD) δ 8.36 (d, J=2.4 Hz, 1H), 7.80 (dd, J=8.4, 2.0 Hz, 1H), 7.73 (d, J=8.4 Hz, 1H), 7.39 (m, 2H), 7.10 (dd, J=5.2, 3.6 Hz, 1H), 3.19 (t, J=8.0 Hz, 2H), 1.74 (m, 2H), 1.38 (m, 2H), 1.22 (m, 8H), 0.85 (t, J=7.2 Hz, 3H).

(7r) 8.86 (s, 1H), 8.55 (d, J=4.4 Hz, 1H), 8.42 (s, 1H), 8.17 (d, J=8.0 Hz, 1H), 7.90 (m, 2H), 7.57 (m, 1H), 3.25 (t, J=8.0 Hz, 2H), 1.77 (m, 2H), 1.37 (m, 2H), 1.24 (m, 8H), 0.86 (t, J=7.2 Hz, 3H).

(7s) ¹H NMR (MeOD) δ 8.21 (s, 1H), 7.75 (m, 2H), 7.45 (d, J=1.6 Hz, 2H), 7.32 (t, J=1.6 Hz, 1H), 3.22 (t, J=8.0 Hz, 2H), 1.74 (m, 2H), 1.33 (m, 2H), 1.19 (m, 8H), 0.83 (t, J=7.2 Hz, 3H).

(7t) ¹H NMR (MeOD) δ 8.16 (d, J=2.4 Hz, 1H), 7.74 (d, J=8.8 Hz, 1H), 7.54 (dd, J=8.4, 2.4 Hz, 1H), 7.50 (s, 1H), 7.34 (s, 2H), 3.20 (t, J=8.0 Hz, 2H), 1.75 (m, 2H), 1.34 (m, 2H), 1.20 (m, 8H), 0.84 (t, J=6.8 Hz, 3H).

### Example 8

### In Vitro Testing

The compounds produced as described above were evaluated for their ability to inhibit the acylation of glycerol-3-phosphate in vitro. The acylation reaction between ¹⁴C-labelled glycerol-3-phosphate and palmitoyl-CoA, initiated by adding mtGPAT, was measured in the presence of varying concentrations of the inhibitor by scintillation counting as described in more detail below.

A mitochondrial preparation of glycerol 3-phosphate acyltransferase was added to the incubation mixture containing ¹⁴C-labeled glycerol 3-phosphate, palmitoyl-CoA, and varying inhibitor concentrations to initiate the reaction. After ten min, the reaction was terminated by adding chloroform, methanol, and 1% perchloric acid. Five minutes later, more chloroform and perchloric acid were added, and the upper aqueous layer was removed. After washing three times with 1% perchloric acid, the organic layer was evaporated under nitrogen, and the amount of ¹⁴C present was counted to determine the extent of reaction inhibition. Data points were recorded in triplicate, and IC₅₀ values were calculated based on the amount of test inhibitor necessary to produce 50% of mtGPAT activity observed in the absence of inhibitor but in the presence of DMSO vehicle control.

Results for compounds 5a-f, 13a-f, 15a-i, 17a-f, 21a-c, and 24a-f are summarized in Tables 1-3 below. The results for each of the compounds 4a-t and 7a-t are summarized individually below.

TABLE 1
In Vitro Anti-mtGPAT1 Activity of Sulfonamides 5a-f and 13a-f

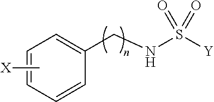

| Compound | X | Y | n | IC₅₀ (μM) ± SD |
|---|---|---|---|---|
| 5a | p-CO₂H | C₅H₁₁ | 1 | 72.0 ± 1.7 |
| 5b | p-CO₂H | C₉H₁₉ | 1 | 43.9 ± 6.3 |
| 5c | m-CO₂H | C₅H₁₁ | 1 | 88.5 ± 1.7 |
| 5d | m-CO₂H | C₉H₁₉ | 1 | 28.5 ± 1.6 |
| 5e | o-CO₂H | C₅H₁₁ | 1 | 61.9 ± 13.5 |
| 5f | o-CO₂H | C₉H₁₉ | 1 | 22.7 ± 1.1 |
| 13a | o-CH₂PO₃H₂ | C₅H₁₁ | 0 | 41.4 ± 8.4 |
| 13b | o-CH₂PO₃H₂ | C₉H₁₉ | 0 | 30.6 ± 6.2 |
| 13c | m-CH₂PO₃H₂ | C₅H₁₁ | 0 | 45.3 ± 9.0 |
| 13d | m-CH₂PO₃H₂ | C₉H₁₉ | 0 | 23.7 ± 0.7 |
| 13e | p-CH₂PO₃H₂ | C₅H₁₁ | 0 | 47.7 ± 9.6 |
| 13f | p-CH₂PO₃H₂ | C₉H₁₉ | 0 | 30.7 ± 5.4 |

Data obtained from benzoic acids 5a-f indicate that in all cases, regardless of the position of the carboxylate with respect to the sulfonamide, the longer C₉ alkyl chain resulted in greater inhibition than the C₅ saturated chain. The most effective orientation between the acid and sulfonamide appeared to be ortho-substitution, as 5f (IC₅₀=22.7 μM) is a better inhibitor than either 5b (IC₅₀=43.9 μM) or 5d (IC₅₀=28.5 μM). The assay data from phosphonic acids 13a-f also indicated that the longer C₉ alkyl chain is more effective. In this series of compounds, however, there is no significant difference in activity between the different orientations of the phosphonic acid and the alkyl sulfonamide moiety. The most active compound of this class was 13d (IC₅₀=23.7 μM), the meta-substituted phosphonic acid, though not by much over 13b (IC₅₀=30.6 μM) and 13f (IC₅₀=30.7 μM).

TABLE 2
In Vitro Anti-mtGPAT1 Activity of Sulfonamides 15a-i and 17a-f

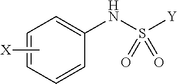

| Compound | X | Y | IC₅₀ (μM) ± SD |
|---|---|---|---|
| 15a | p-CO₂H | C₉H₁₉ | 29.1 ± 4.3 |
| 15b | p-CO₂H | Ph | 41.9 ± 5.3 |
| 15c | p-CO₂H | 4-ClPh | 33.7 ± 1.3 |
| 15d | m-CO₂H | C₉H₁₉ | 24.2 ± 2.9 |
| 15e | m-CO₂H | Ph | 38.3 ± 7.6 |
| 15f | m-CO₂H | 4-ClPh | 23.6 ± 1.2 |
| 15g (C67) | o-CO₂H | C₉H₁₉ | 8.1 ± 0.7 |
| 15h | o-CO₂H | Ph | 40.5 ± 2.6 |
| 15i | o-CO₂H | 4-ClPh | 33.5 ± 2.5 |
| 17a | p-CO₂H | CH₂Ph | 64.5 ± 11.6 |
| 17b | p-CO₂H | C₂H₄Ph | 63.0 ± 12.9 |
| 17c | m-CO₂H | CH₂Ph | 52.1 ± 9.0 |
| 17d | m-CO₂H | C₂H₄Ph | 50.3 ± 4.4 |
| 17e | o-CO₂H | CH₂Ph | 40.7 ± 1.2 |
| 17f | o-CO₂H | C₂H₄Ph | 46.4 ± 4.5 |

The distance between the benzene ring and the sulfonamide sulfur does not appear to have a significant effect on the inhibitory activity of these compounds, as there is effectively no difference between one methylene and two methylene linkers. It is apparent, however, that the ortho-substituted compounds containing these linker methylenes (17e-f) are more effective than the other substituted benzoic acids (17a-d). For the meta- and para-compounds, inhibitory activity is greater when the benzene ring is directly attached to the sulfur, although the ortho-compounds are all similar. The addition of a para-chloride on the benzene ring leads to slight increases in activity for the para- (15c), meta- (15f), and ortho-compounds (15i). Compounds 15a, 15d, and 15g were easily obtainable targets, which allowed for examination of the effect of the methylene linker between the benzene ring and the sulfonamide in 5a-f. For every substitution, these compounds were the most effective GPAT inhibitors, with the ortho-compound (15g, C67) demonstrating the greatest activity (IC₅₀=8.1 μM). Based on these results, a long alkyl chain is preferable to a simple benzene ring.

TABLE 3
In Vitro Anti-mtGPAT1 Activity of Sulfonamides 21a-c and 24a-f

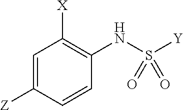

| Compound | X | Y | Z | IC₅₀ (μM) ± SD |
|---|---|---|---|---|
| 21a | p-PO₃H₂ | C₈H₁₇ | H | 33.3 ± 3.8 |
| 21b | m-PO₃H₂ | C₈H₁₇ | H | 25.3 ± 5.4 |
| 21c | o-PO₃H₂ | C₈H₁₇ | H | 25.7 ± 2.5 |
| 24a | CO₂H | CH₃ | H | 28.6 ± 4.6 |
| 24b | CO₂H | C₁₄H₂₉ | H | 6.9 ± 0.5 |
| 24c | CO₂H | C₁₆H₃₃ | H | 7.8 ± 0.8 |
| 24d | CO₂H | C₉H₁₉ | Cl | 11.5 ± 0.7 |
| 24e | CO₂H | C₈H₁₇ | OH | 38.2 ± 4.1 |
| 24f | CO₂H | C₈H₁₇ | F | 29.5 ± 2.6 |

In view of the increased inhibitory activity of 15g, two other compound series were prepared. The first, 21a-c, probes the effectiveness of an aryl phosphonic acid in place of the benzoic acid moiety. In vitro, the ortho-substituted acid (21c) is less active than 15g, and substitution of the phosphonic acid moiety does not appear to significantly affect activity (Table 3). The other compounds produced (24a-f) indicate the importance of chain length of the alkyl sulfonamide, as well as the effect of adding heteroatoms para- to the sulfonamide. It appears that the longer chain is very important to the activity of these compounds, as a C₁-chain (24a) results in significantly less in vitro activity than the C₉ chain. Compounds 24b and 24c were produced to determine if the naturally-favored C₁₆ chain is preferred in these compounds over other chain lengths, including the C₁₄ chain. In this case, there is no observed preference for the C₁₆ compound over other long chains, in contrast to that observed with the natural acyl-CoA substrates.

Results for compounds 4a-t and 7a-t, which were developed using the methods described above, include the following:

| | | | | |
|---|---|---|---|---|
| 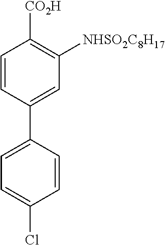 | FAS (IC₅₀) Not Tested CPT I Stim Not Tested FAO SC 150 Neg SA/MH (MIC) ug/ml | ¹⁴C (IC₅₀) Not Tested FAO Max 107% at 6.25 ug/ml SA/Tsoy(MIC) ug/ml | Weight Loss Not Tested EF/MH (MIC) ug/ml | GPAT IC₅₀ 7.8 ± 1.1 ug/ml EF/Tsoy(MIC) ug/ml |
| 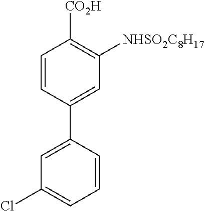 | FAS (IC₅₀) Not Tested CPT I Stim Not Tested FAO SC 150 Neg SA/MH (MIC) ug/ml | ¹⁴C (IC₅₀) Not Tested FAO Max 115% at 6.25 ug/ml SA/Tsoy(MIC) ug/ml | Weight Loss Not Tested EF/MH (MIC) ug/ml | GPAT IC₅₀ 6.8 ± 0.5 ug/ml EF/Tsoy(MIC) ug/ml |
| 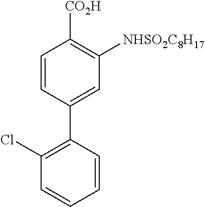 | FAS (IC₅₀) Not Tested CPT I Stim Not Tested FAO SC 150 Neg SA/MH (MIC) ug/ml | ¹⁴C (IC₅₀) Not Tested FAO Max 89% at 1.56 ug/ml SA/Tsoy(MIC) ug/ml | Weight Loss Not Tested EF/MH (MIC) ug/ml | GPAT IC₅₀ 9.8 ± 0.9 ug/ml EF/Tsoy(MIC) ug/ml |
| 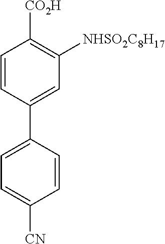 | FAS (IC₅₀) Not Tested CPT I Stim Not Tested FAO SC 150 Neg SA/MH (MIC) ug/ml | ¹⁴C (IC₅₀) Not Tested FAO Max 83% at 0.098 ug/ml SA/Tsoy(MIC) ug/ml | Weight Loss Not Tested EF/MH (MIC) ug/ml | GPAT IC₅₀ 8.3 ± 0.4 ug/ml EF/Tsoy(MIC) ug/ml |
| 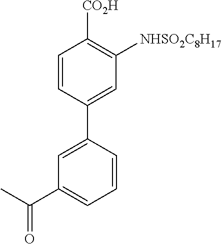 | FAS (IC₅₀) Not Tested CPT I Stim Not Tested FAO SC 150 Neg SA/MH (MIC) ug/ml | ¹⁴C (IC₅₀) Not Tested FAO Max 98% at 6.25 ug/ml SA/Tsoy(MIC) ug/ml | Weight Loss Not Tested EF/MH (MIC) ug/ml | GPAT IC₅₀ 12.6 ± 2.1 ug/ml EF/Tsoy(MIC) ug/ml |
| 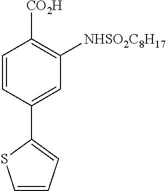 | FAS (IC₅₀) Not Tested CPT I Stim Not Tested FAO SC 150 Neg SA/MH (MIC) ug/ml | ¹⁴C (IC₅₀) Not Tested FAO Max 83% at 0.098 ug/ml SA/Tsoy(MIC) ug/ml | Weight Loss Not Tested EF/MH (MIC) ug/ml | GPAT IC₅₀ 8.9 ± 1.1 ug/ml EF/Tsoy(MIC) ug/ml |
| 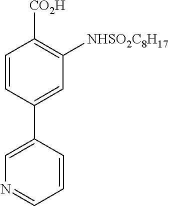 | FAS (IC₅₀) Not Tested CPT I Stim Not Tested FAO SC 150 Neg SA/MH (MIC) ug/ml | ¹⁴C (IC₅₀) Not Tested FAO Max 92% at 0.395 ug/ml SA/Tsoy(MIC) ug/ml | Weight Loss Not Tested EF/MH (MIC) ug/ml | GPAT IC₅₀ 25.7 ± 0.4 ug/ml EF/Tsoy(MIC) ug/ml |
| 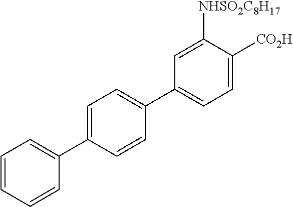 | FAS (IC₅₀) Not Tested CPT I Stim Not Tested FAO SC 150 Neg SA/MH (MIC) ug/ml | ¹⁴C (IC₅₀) Not Tested FAO Max 82% at 0.098 ug/ml SA/Tsoy(MIC) ug/ml | Weight Loss Not Tested EF/MH (MIC) ug/ml | GPAT IC₅₀ 8.1 ± 1.0 ug/ml EF/Tsoy(MIC) ug/ml |
| 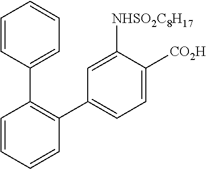 | FAS (IC₅₀) Not Tested CPT I Stim Not Tested FAO SC 150 Neg SA/MH (MIC) ug/ml | ¹⁴C (IC₅₀) Not Tested FAO Max 113% at 6.25 ug/ml SA/Tsoy(MIC) ug/ml | Weight Loss Not Tested EF/MH (MIC) ug/ml | GPAT IC₅₀ 8.4 ± 0.2 ug/ml EF/Tsoy(MIC) ug/ml |
| 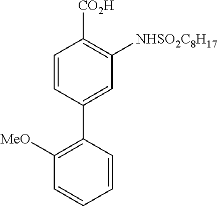 | FAS (IC₅₀) Not Tested CPT I Stim Not Tested FAO SC 150 Neg SA/MH (MIC) ug/ml | ¹⁴C (IC₅₀) Not Tested FAO Max 112% at 6.25 ug/ml SA/Tsoy(MIC) ug/ml | Weight Loss Not Tested EF/MH (MIC) ug/ml | GPAT IC₅₀ 7.4 ± 0.2 ug/ml EF/Tsoy(MIC) ug/ml |
| 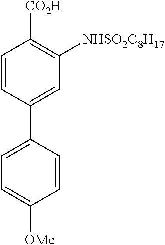 | FAS (IC₅₀) Not Tested CPT I Stim Not Tested FAO SC 150 Neg SA/MH (MIC) ug/ml | ¹⁴C (IC₅₀) Not Tested FAO Max 101% at 1.56 ug/ml SA/Tsoy(MIC) ug/ml | Weight Loss Not Tested EF/MH (MIC) ug/ml | GPAT IC₅₀ 6.7 ± 0.2 ug/ml EF/Tsoy(MIC) ug/ml |
| 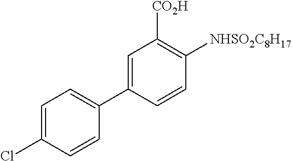 | FAS (IC₅₀) Not Tested CPT I Stim Not Tested FAO SC 150 Neg SA/MH (MIC) ug/ml | ¹⁴C (IC₅₀) Not Tested FAO Max 86% at 1.56 ug/ml SA/Tsoy(MIC) ug/ml | Weight Loss Not Tested EF/MH (MIC) ug/ml | GPAT IC₅₀ 5.7 ± 0.2 ug/ml EF/Tsoy(MIC) ug/ml |
| 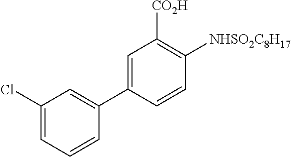 | FAS (IC₅₀) Not Tested CPT I Stim Not Tested FAO SC 150 Neg SA/MH (MIC) ug/ml | ¹⁴C (IC₅₀) Not Tested FAO Max 126% at 0.098 ug/ml SA/Tsoy(MIC) ug/ml | Weight Loss Not Tested EF/MH (MIC) ug/ml | GPAT IC₅₀ 5.5 ± 0.3 ug/ml EF/Tsoy(MIC) ug/ml |
| 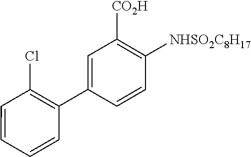 | FAS (IC₅₀) Not Tested CPT I Stim Not Tested FAO SC 150 Neg SA/MH (MIC) ug/ml | ¹⁴C (IC₅₀) Not Tested FAO Max 124% at 0.395 ug/ml SA/Tsoy(MIC) ug/ml | Weight Loss Not Tested EF/MH (MIC) ug/ml | GPAT IC₅₀ 6.1 ± 0.3 ug/ml EF/Tsoy(MIC) ug/ml |
| 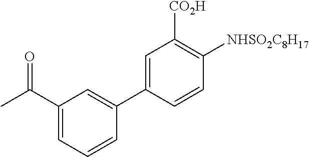 | FAS (IC₅₀) Not Tested CPT I Stim Not Tested FAO SC 150 Neg SA/MH (MIC) ug/ml | ¹⁴C (IC₅₀) Not Tested FAO Max 104% at 0.098 ug/ml SA/Tsoy(MIC) ug/ml | Weight Loss Not Tested EF/MH (MIC) ug/ml | GPAT IC₅₀ 12.1 ± 1.3 ug/ml EF/Tsoy(MIC) ug/ml |
| 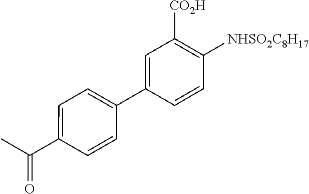 | FAS (IC₅₀) Not Tested CPT I Stim Not Tested FAO SC 150 Neg SA/MH (MIC) ug/ml | ¹⁴C (IC₅₀) Not Tested FAO Max 89% at 100 ug/ml SA/Tsoy(MIC) ug/ml | Weight Loss Not Tested EF/MH (MIC) ug/ml | GPAT IC₅₀ 303 ± 47 ug/ml EF/Tsoy(MIC) ug/ml |
| 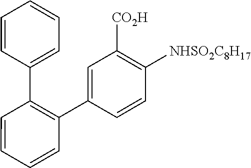 | FAS (IC₅₀) Not Tested CPT I Stim Not Tested FAO SC 150 Neg SA/MH (MIC) ug/ml | ¹⁴C (IC₅₀) Not Tested FAO Max 95% at 0.395 ug/ml SA/Tsoy(MIC) ug/ml | Weight Loss Not Tested EF/MH (MIC) ug/ml | GPAT IC₅₀ 6.3 ± 0.3 ug/ml EF/Tsoy(MIC) ug/ml |
| 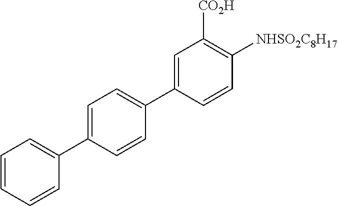 | FAS (IC₅₀) Not Tested CPT I Stim Not Tested FAO SC 150 Neg SA/MH (MIC) ug/ml | ¹⁴C (IC₅₀) Not Tested FAO Max 119% at 0.098 ug/ml SA/Tsoy(MIC) ug/ml | Weight Loss Not Tested EF/MH (MIC) ug/ml | GPAT IC₅₀ 30.6 ± 0.8 ug/ml EF/Tsoy(MIC) ug/ml |
| 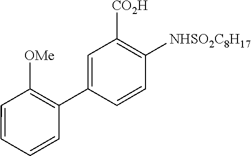 | FAS (IC₅₀) Not Tested CPT I Stim Not Tested FAO SC 150 Neg SA/MH (MIC) ug/ml | ¹⁴C (IC₅₀) Not Tested FAO Max 91% at 0.098 ug/ml SA/Tsoy(MIC) ug/ml | Weight Loss Not Tested EF/MH (MIC) ug/ml | GPAT IC₅₀ 9.8 ± 0.7 ug/ml EF/Tsoy(MIC) ug/ml |
| 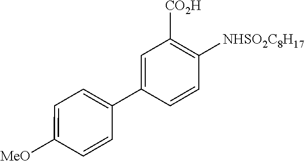 | FAS (IC₅₀) Not Tested CPT I Stim Not Tested FAO SC 150 Neg SA/MH (MIC) ug/ml | ¹⁴C (IC₅₀) Not Tested FAO Max 89% at 0.395 ug/ml SA/Tsoy(MIC) ug/ml | Weight Loss Not Tested EF/MH (MIC) ug/ml | GPAT IC₅₀ 8.0 ± 1.0 ug/ml EF/Tsoy(MIC) ug/ml |
| 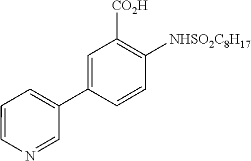 | FAS (IC₅₀) Not Tested CPT I Stim Not Tested FAO SC 150 Neg SA/MH (MIC) ug/ml | ¹⁴C (IC₅₀) Not Tested FAO Max 104% at 1.56 ug/ml SA/Tsoy(MIC) ug/ml | Weight Loss Not Tested EF/MH (MIC) ug/ml | GPAT IC₅₀ 29.8 ± 2.6 ug/ml EF/Tsoy(MIC) ug/ml |
| 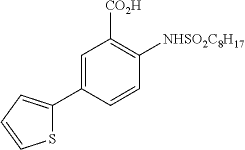 | FAS (IC₅₀) Not Tested CPT I Stim Not Tested FAO SC 150 Neg SA/MH (MIC) ug/ml | ¹⁴C (IC₅₀) Not Tested FAO Max 87% at 0.098 ug/ml SA/Tsoy(MIC) ug/ml | Weight Loss Not Tested EF/MH (MIC) ug/ml | GPAT IC₅₀ 8.8 ± 0.8 ug/ml EF/Tsoy(MIC) ug/ml |
| 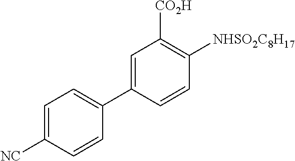 | FAS (IC₅₀) Not Tested CPT I Stim Not Tested FAO SC 150 Neg SA/MH (MIC) ug/ml | ¹⁴C (IC₅₀) Not Tested FAO Max 100% at 0.395 ug/ml SA/Tsoy(MIC) ug/ml | Weight Loss Not Tested EF/MH (MIC) ug/ml | GPAT IC₅₀ 10.2 ± 0.9 ug/ml EF/Tsoy(MIC) ug/ml |
| 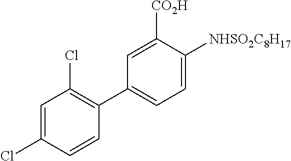 | FAS (IC₅₀) Not Tested CPT I Stim Not Tested FAO SC 150 Neg SA/MH (MIC) ug/ml | ¹⁴C (IC₅₀) Not Tested FAO Max 90% at 0.098 ug/ml SA/Tsoy(MIC) ug/ml | Weight Loss Not Tested EF/MH (MIC) ug/ml | GPAT IC₅₀ 7.9 ± 0.8 ug/ml EF/Tsoy(MIC) ug/ml |
| 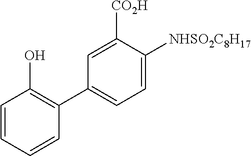 | FAS (IC₅₀) Not Tested CPT I Stim Not Tested FAO SC 150 Neg SA/MH (MIC) ug/ml | ¹⁴C (IC₅₀) Not Tested FAO Max 109% at 0.098 ug/ml SA/Tsoy(MIC) ug/ml | Weight Loss Not Tested EF/MH (MIC) ug/ml | GPAT IC₅₀ 25.7 ± 3.2 ug/ml EF/Tsoy(MIC) ug/ml |
| 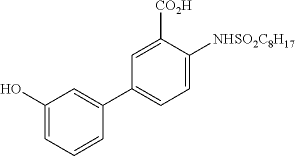 | FAS (IC₅₀) Not Tested CPT I Stim Not Tested FAO SC 150 SA/MH (MIC) ug/ml | ¹⁴C (IC₅₀) Not Tested FAO Max % at ug/ml SA/Tsoy(MIC) ug/ml | Weight Loss Not Tested EF/MH (MIC) ug/ml | GPAT IC₅₀ 23.4 ± 1.0 ug/ml EF/Tsoy(MIC) ug/ml |
| 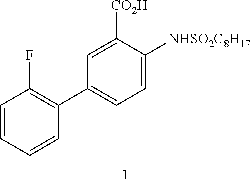 | FAS (IC₅₀) Not Tested CPT I Stim Not Tested FAO SC 150 Neg SA/MH (MIC) ug/ml | ¹⁴C (IC₅₀) Not Tested FAO Max 103% at 1.56 ug/ml SA/Tsoy(MIC) ug/ml | Weight Loss Not Tested EF/MH (MIC) ug/ml | GPAT IC₅₀ 12.7 ± 0.7 ug/ml EF/Tsoy(MIC) ug/ml |
| 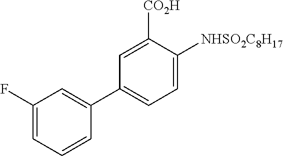 | FAS (IC₅₀) Not Tested CPT I Stim Not Tested FAO SC 150 Neg SA/MH (MIC) ug/ml | ¹⁴C (IC₅₀) Not Tested FAO Max 102% at 6.25 ug/ml SA/Tsoy(MIC) ug/ml | Weight Loss Not Tested EF/MH (MIC) ug/ml | GPAT IC₅₀ 21.2 ± 3.1 ug/ml EF/Tsoy(MIC) ug/ml |
| 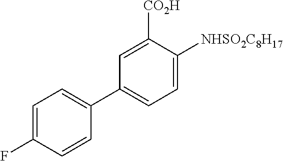 | FAS (IC₅₀) Not Tested CPT I Stim Not Tested FAO SC 150 SA/MH (MIC) ug/ml | ¹⁴C (IC₅₀) Not Tested FAO Max % at ug/ml SA/Tsoy(MIC) ug/ml | Weight Loss Not Tested EF/MH (MIC) ug/ml | GPAT IC₅₀ 8.4 ± 1.7 ug/ml EF/Tsoy(MIC) ug/ml |
| 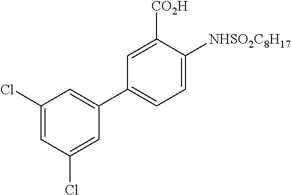 | FAS (IC₅₀) Not Tested CPT I Stim Not Tested FAO SC 150 SA/MH (MIC) ug/ml | ¹⁴C (IC₅₀) Not Tested FAO Max % at ug/ml SA/Tsoy(MIC) ug/ml | Weight Loss Not Tested EF/MH (MIC) ug/ml | GPAT IC₅₀ 8.7 ± 1.4 ug/ml EF/Tsoy(MIC) ug/ml |
| 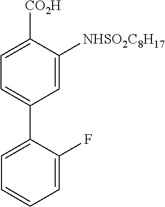 | FAS (IC₅₀) Not Tested CPT I Stim Not Tested FAO SC 150 SA/MH (MIC) ug/ml | ¹⁴C (IC₅₀) Not Tested FAO Max % at ug/ml SA/Tsoy(MIC) ug/ml | Weight Loss Not Tested EF/MH (MIC) ug/ml | GPAT IC₅₀ 22.7 ± 1.0 ug/ml EF/Tsoy(MIC) ug/ml |
| 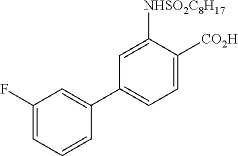 | FAS (IC₅₀) Not Tested CPT I Stim Not Tested FAO SC 150 SA/MH (MIC) ug/ml | ¹⁴C (IC₅₀) Not Tested FAO Max % at ug/ml SA/Tsoy(MIC) ug/ml | Weight Loss Not Tested EF/MH (MIC) ug/ml | GPAT IC₅₀ 11.7 ± 0.8 ug/ml EF/Tsoy(MIC) ug/ml |
| 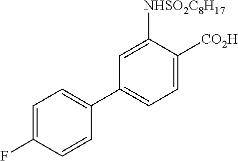 | FAS (IC₅₀) Not Tested CPT I Stim Not Tested FAO SC 150 SA/MH (MIC) ug/ml | ¹⁴C (IC₅₀) Not Tested FAO Max % at ug/ml SA/Tsoy(MIC) ug/ml | Weight Loss Not Tested EF/MH (MIC) ug/ml | GPAT IC₅₀ 10.3 ± 0.9 ug/ml EF/Tsoy(MIC) ug/ml |
| 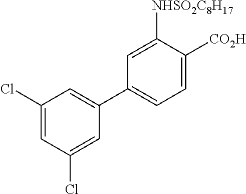 | FAS (IC₅₀) Not Tested CPT I Stim Not Tested FAO SC 150 SA/MH (MIC) ug/ml | ¹⁴C (IC₅₀) Not Tested FAO Max % at ug/ml SA/Tsoy(MIC) ug/ml | Weight Loss Not Tested EF/MH (MIC) ug/ml | GPAT IC₅₀ 8.8 ± 2.4 ug/ml EF/Tsoy(MIC) ug/ml |
| 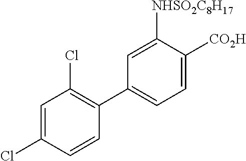 | FAS (IC₅₀) Not Tested CPT I Stim Not Tested FAO SC 150 SA/MH (MIC) ug/ml | ¹⁴C (IC₅₀) Not Tested FAO Max % at ug/ml SA/Tsoy(MIC) ug/ml | Weight Loss Not Tested EF/MH (MIC) ug/ml | GPAT IC₅₀ 8.4 ± 1.9 ug/ml EF/Tsoy(MIC) ug/ml |
| 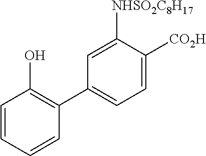 | FAS (IC₅₀) Not Tested CPT I Stim Not Tested FAO SC 150 SA/MH (MIC) ug/ml | ¹⁴C (IC₅₀) Not Tested FAO Max % at ug/ml SA/Tsoy(MIC) ug/ml | Weight Loss Not Tested EF/MH (MIC) ug/ml | GPAT IC₅₀ 25.4 ± 1.6 ug/ml EF/Tsoy(MIC) ug/ml |
| 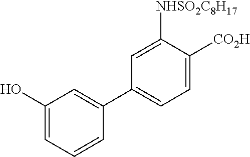 | FAS (IC₅₀) Not Tested CPT I Stim Not Tested FAO SC 150 SA/MH (MIC) ug/ml | ¹⁴C (IC₅₀) Not Tested FAO Max % at ug/ml SA/Tsoy(MIC) ug/ml | Weight Loss Not Tested EF/MH (MIC) ug/ml | GPAT IC₅₀ 22.5 ± 0.5 ug/ml EF/Tsoy(MIC) ug/ml |
| 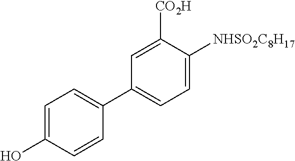 | FAS (IC₅₀) Not Tested CPT I Stim Not Tested FAO SC 150 SA/MH (MIC) ug/ml | ¹⁴C (IC₅₀) Not Tested FAO Max % at ug/ml SA/Tsoy(MIC) ug/ml | Weight Loss Not Tested EF/MH (MIC) ug/ml | GPAT IC₅₀ 24.7 ± 1.7 ug/ml EF/Tsoy(MIC) ug/ml |
| 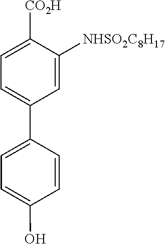 | FAS (IC₅₀) Not Tested CPT I Stim Not Tested FAO SC 150 SA/MH (MIC) ug/ml | ¹⁴C (IC₅₀) Not Tested FAO Max % at ug/ml SA/Tsoy(MIC) ug/ml | Weight Loss Not Tested EF/MH (MIC) ug/ml | GPAT IC₅₀ 26.8 ± 1.4 ug/ml EF/Tsoy(MIC) ug/ml |
| 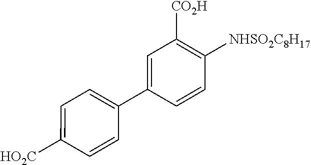 | FAS (IC₅₀) Not Tested CPT I Stim Not Tested FAO SC 150 SA/MH (MIC) ug/ml | ¹⁴C (IC₅₀) Not Tested FAO Max % at ug/ml SA/Tsoy(MIC) ug/ml | Weight Loss Not Tested EF/MH (MIC) ug/ml | GPAT IC₅₀ ug/ml EF/Tsoy(MIC) ug/ml |
| 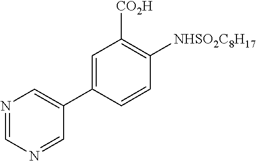 | FAS (IC₅₀) Not Tested CPT I Stim Not Tested FAO SC 150 SA/MH (MIC) ug/ml | ¹⁴C (IC₅₀) Not Tested FAO Max % at ug/ml SA/Tsoy(MIC) ug/ml | Weight Loss Not Tested EF/MH (MIC) ug/ml | GPAT IC₅₀ ug/ml EF/Tsoy(MIC) ug/ml |

### Example 9

### In Vivo Testing

Experimental Procedures

DIO and lean mouse models. All animal experimentation was done in accordance with guidelines on animal care and use as established by the Johns Hopkins University School of Medicine IACUC. DIO C57BL6J male mice were obtained from Jackson Laboratory (Bar Harbor, ME) and fed a synthetic diet comprised of 60% calories from fat, 20% from carbohydrate, and 20% from protein (5.2 kcal/g) post-weaning through the experimental procedures (D12492i, Research Diets, Inc., New Brunswick, N.J.). For lean animal studies, twelve-week old C57BL6J male mice (Jackson Laboratory, Bar Harbor, Me.) were fed rodent chow comprised of 13% calories from fat, 58% from carbohydrate, and 29% from protein (4.1 kcal/g) (Prolab RMH 2500, PMI Nutrition International Inc., Brentwood, Mo.). Mice were maintained in 12 hr light-dark cycle at 25° C. for 1 week for acclimatization prior to treatment. In all studies, FSG67 (FASgen, Inc., Baltimore, Md.) was dissolved in RPMI 1640 (Invitrogen, Carlsbad, Calif.).

For acute studies, 6 DIO or lean mice were treated with a single dose of FSG67 (20 mg/kg, i.p.) approximately 3 hrs past lights-on. Animal weights and food consumption were measured 18 h after treatment. Following euthanization, the hypothalmuses were harvested to measure orexigenic and anorexigenic neuropeptide gene expression. In the chronic studies, DIO mice, 4-10 animals per group, were treated daily with FSG67 (5 mg/kg, i.p.) or with RMPI vehicle for the days indicated. Body weight and food intake were measured daily. In one study, a cohort of mice was pair-fed with amounts consumed by the FSG67-treated animals and mice were monitored with indirect calorimetry (Oxymax Equal Flow System®, Columbus Instruments, Columbus, Ohio). Measurements of VO2 (ml/kg/hr) and VCO2 (ml/kg/hr) were performed and recorded every 15 min. The respiratory exchange ratio (RER) was calculated by Oxymax software, version 5.9, and is defined as ratio of VCO2 to VO2 33. After completion of the treatment course, animals were euthanized by CO₂ inhalation 4 hrs following the final dose of FSG67. Tissues were harvested immediately for RNA extraction; serum was collected and analyzed for glucose, cholesterol, and triglyceride measurements (Bioanalytics, Gaithersburg, Md.). Fresh liver tissue was snap frozen in liquid N2, sectioned, and stained with hematoxylin and Oil Red O to visualize triglyceride droplets.

Chronic lateral cerebroventricle cannulas. For experiments requiring intracerebroventricular (i.c.v.) administration of compounds, mice were outfitted unilaterally with chronic indwelling cannulas aimed at the lateral cerebroventricle. After mice recovered from surgeries for one week, cannula placements were assessed by measuring food intake in response to i.c.v. neuropeptide Y (NPY, American Peptide Co., CA). Mice were given NPY (0.25 ηmol/2 μl injection) or sterile 0.9% saline vehicle via the i.c.v. cannula, and allowed 1-h access to grain-based pellets during the light phase. Mice that ate at least 0.5 g of food after NPY were used in the experiments. Eleven mice were given a 2 μL injection of RPMI-1640 without glucose (Cambrex, Md.) for vehicle control. Three days later, six mice received a 100 nmole dose of FSG67 in the vehicle while 5 mice received 320 nmoles of compound.

Q-NMR assessment of adiposity. Following 10 days of FSG67 treatment or vehicle by ip administration, the DIO mice were euthanized and carcasses were stored at −80° C. Carcasses were thawed for Q-NMR analysis. Measurement of fat, lean, and water mass was performed using an EchoMRI-100™ (Echo Medical Systems, Houston, Tex.) in the Molecular and Comparative Pathobiology Phenotyping Core.

Conditioned taste aversion. Ten days before testing, eighteen male C57/BL6 mice were placed on a schedule of 2 h daytime access to water. On the test day, mice were divided into three groups and were given access to 0.15% sodium saccharin rather than water for 30 min. Immediately after saccharin access, mice were injected ip with RPMI vehicle or FSG67 (5 and 20 mg/kg body wt) and were allowed water access for the remaining 90 min. Twenty-four hours later, mice were given 2h access to a two-bottle choice test of 0.15% saccharin vs. water. Intakes of both solutions were recorded, and data were expressed as saccharin preference (100× saccharin intake/saccharin intake+water intake).

Real-time RT-PCR. Hypothalamus, liver, and WAT of DIO and lean mice were harvested and immediately frozen in liquid nitrogen. Total RNA was isolated and real-time quantitative RT-PCR was performed as previously described (13). Gene-specific primer pairs were designed using Primer3 software (http://www-genome.wi.mit.edu/cgi-bin/primer/primer3_www.cgi/). The sequences of the primer pairs are listed in Supplemental Data Table 1.

3T3-L1 Adipocytes 3T3-L1 cells were differentiated into adipocytes as described 34. Seven days post-differentiation, cells were treated with FSG67 at indicated concentrations for 18 h, then labeled with [14C]palmitate for 2 h. Following Folch extraction, lipids were subjected to polar and non-polar thin-layer chromatography 35. Triglyceride and phosphatidylcholine fractions were quantified with phosphorimaging (Storm 840, Molecular Dynamics, Piscataway, N.J.).

Statistical analysis. All data are presented as means±standard error of the mean. IC50 determinations were performed with linear regression. Two-tailed unpaired t-tests or two-way ANOVA tests were performed as indicated using Prism 4.0 (Graph Pad Software, San Diego, Calif.).

FSG67 Reduces Acylglyceride Synthesis in Mouse 3T3-L1 Adipocytes.

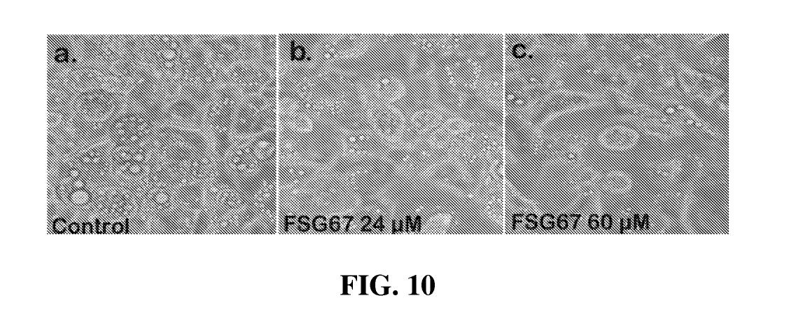

Mouse 3T3-L1 adipocytes were used to test the effect FSG67 on acylglyceride synthesis in vitro. 3T3-L1 adipocytes, at 7 days post-differentiation, were treated with FSG67 at concentrations of 7.6 μM to 61 μM (2.5-20 μg/ml) and the IC50 values for inhibition of triglyceride and phosphatidylcholine synthesis were determined using linear regression. The IC50 values were 33.9 μM for cellular triglyceride synthesis (p=0.023, r2=0.86, n=3) and 36.3 μM for phosphatidylcholine synthesis (p=0.015, r2=0.89, n=3). As phosphatidylcholine was the predominant phospholipid synthesized in the 3T3-L1 adipocytes, it is representative of overall cellular phospholipid synthesis. These IC50 values are similar to the reported IC50 value of 24.7 μM for mouse mitochondrial GPAT activity 12. Consistent with its inhibition of acylglyceride synthesis, FIG. 10 shows the dose-dependent reduction of triglyceride accumulation in 3T3-L1 adipocytes 48 h following FSG67 treatment. Note the decrease in lipid droplets in the FSG67 treated cells compared to vehicle treated controls. Thus, FSG67 inhibits cellular acylglyceride synthesis with an IC50 similar to its inhibition of GPAT activity in mitochondrial preparations. In keeping with these biochemical observations, FSG67 substantially reduced triglyceride accumulation in cultured adipocytes. Taken together, these results demonstrate that FSG67 inhibits cellular GPAT activity.

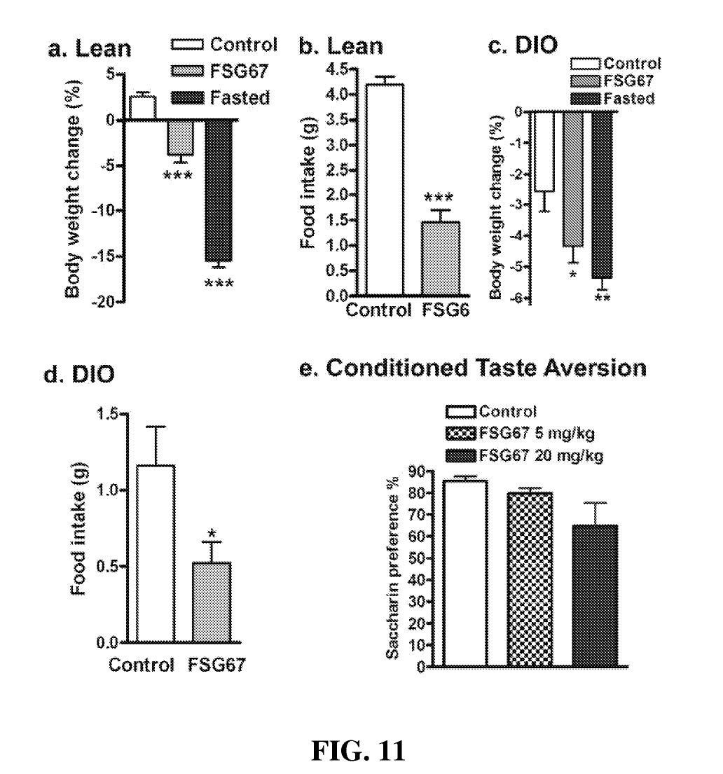

Acute FSG67 treatment of lean and DIO mice reduced body weight, and decreased food consumption without conditioned taste aversion. Since FSG67 reduced acylglyceride synthesis in vitro, we tested both lean and DIO mice with a single dose of FSG67 (20 mg/kg i.p.) to examine the acute effect on animal weight and feeding behavior. In addition, we performed conditioned taste aversion (CTA) testing to determine if FSG67 triggers a CTA response that might suggest malaise as the cause of reduced food intake. Eight DIO and lean mice were treated with FSG67 at the beginning of dark cycle. Within 24 h, the lean mice injected with FSG67 lost 3.7±0.9% (1.0±0.2 g) of body mass while fasted mice lost 15.5±0.7% (3.9±0.2 g) (FIG. 11a). The reduction in body mass of both groups was significant compared to vehicle controls which gained 2.5±0.5% (0.6±0.1g) (p<0.0001, 2-tailed t-test). FSG67 treatment also reduced food intake to 33% of vehicle control (p<0.0001 2-tailed t-test) (FIG. 11b).

GPAT inhibition with FSG67 decreased body weight in DIO mice consuming a high fat diet. FSG67 treated DIO mice lost 4.3±0.5% (1.7±0.2 g) of body mass versus a 5.3±0.4% (2.1±0.2 g) loss for fasted mice (FIG. 11c). Compared to the vehicle control mice which lost 2.5±0.6% (1.0±0.2 g) the weight loss was significant in both the FSG67 treated (p=0.026, 2-tailed t-test) and fasted mice (p=0.002, 2-tailed t-test). FSG67 significantly reduced food consumption in the DIO mice to 41.6% of vehicle control (FIG. 11d). While the average food intake between the DIO and lean vehicle control groups is substantially different (1.2 and 4.2 g, respectively) (p<0.0001, 2-tailed t-test), the relative reduction of food intake following FSG67 treatment is not different between the DIO (41.6% of vehicle control) and lean mice (33% of vehicle control) (p=0.19, Fisher's exact test). CTA testing in groups of 8 lean mice using a two bottle choice paradigm showed that FSG67 failed to produce a significant reduction in saccharin intake at 5 mg/kg (p=0.12) or 20 mg/kg (p=0.10, 2-tailed t-test). Thus, the reduction in food intake from FSG67 was not due to sickness behavior (FIG. 11e). No overt toxicity was noted from the FSG67 treatment of the lean or DIO mice. These data demonstrate a clear anorexigenic effect of pharmacological GPAT inhibition in both lean and DIO mice with accompanying reduction in animal weight.

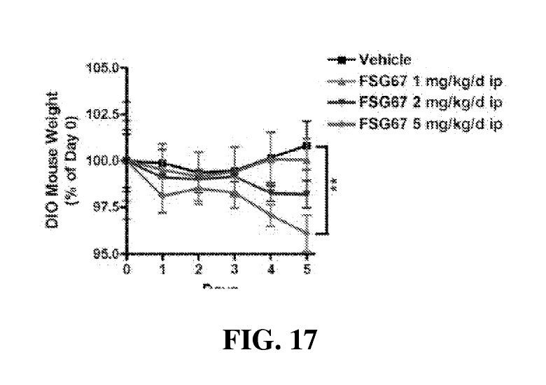

Chronic FSG67 treatment of DIO mice reversibly reduced body weight and food consumption, and increased fatty acid oxidation. To determine the dose of FSG67 suitable for chronic treatment, we performed a 5-day dose ranging study in DIO mice, four per group, with daily intraperitoneal doses of 1, 2, and 5 mg/kg (FIG. 17). The 5 mg/kg dose led to significant weight loss of 3.9% compared to vehicle controls (p=0.008, 2-way ANOVA). This dose was chosen for the subsequent chronic treatment experiments.

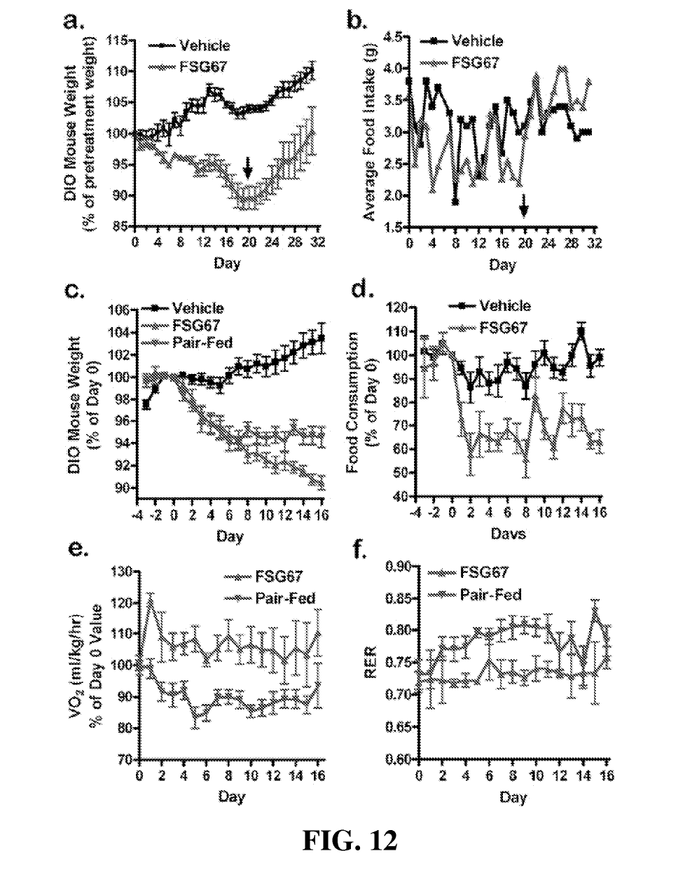

The first chronic treatment experiment was designed to test if weight loss induced by FSG67 was reversible. Four DIO mice per group were treated with FSG67 or vehicle for 20 days. For the entire 32 d trial, weight and food consumption were recorded daily until the FSG67 treated animals regained their original weight. During FSG67 treatment (days 0-20), the mice lost 10.3±0.6% of their body mass while controls gained 4.0±0.5% (p<0.0001, 2-way ANOVA) (FIG. 12a). Average food consumption was reduced during FSG67 treatment (2.6±0.1 g/d, days 1-20) compared to vehicle controls (3.1±0.1 g) (p=0.0008, 2-way ANOVA) (FIG. 12b). Following cessation of treatment, food consumption increased in the FSG67 treatment group to an average of 3.5±0.1 g/d (days 21-32) representing a significant increase in food intake compared to vehicle controls 3.2±0.1 g/d (p=0.006, 2-way ANOVA). The FSG67 treated animals achieved their average pre-treatment weight 11 days following termination of treatment (FIG. 12a).

In the second chronic treatment study, indirect calorimetry was utilized to study changes in metabolism during GPAT inhibition. DIO mice (8 per group) were treated with FSG67 (5 mg/kg, ip), or pair-fed to FSG67 treated animals. Indirect calorimetry was utilized to measure changes in oxygen consumption (VO2) and respiratory exchange ratio (RER) between pair-fed and treated animals. After 16 days of treatment, the FSG67 treated mice lost 9.5±0.6% of body mass, pair-fed lost 5.5±0.9%, while vehicle controls gained 3.5±1.3% (FIG. 12c). The weight loss in the FSG67 treated animals was significant compared to both vehicle controls and pair-fed animals (p<0.0001, 2-way ANOVA). FSG67 treatment again significantly reduced food consumption by 33%, 2.0±0.1 g/d in the FSG67 treated group compared to 3.1±0.1 g/d for vehicle controls (p<0.0001, 2-way ANOVA) (FIG. 12d). FSG67 treatment increased the average VO2 to 106.5±1.1% of pre-treatment value. This value was significantly increased compared to pair-fed mice, which displayed a reduction in VO2 to 89.9±1.1% of the pre-treatment value (p<0.0001, 2-way ANOVA) (FIG. 12e). RER was reduced in FSG67 treated mice (0.732±0.002) compared to pair-fed (0.782±0.006) (p<0.0001, 2-way ANOVA) (FIG. 12f) indicating increased use of fatty acids for fuel in the FSG67 treated animals. The combination of increased VO2 and reduced RER in the FSG67 treated animals are consistent with increased fatty acid oxidation and energy utilization which likely contribute to their reduced body mass compared to the pair-fed controls.

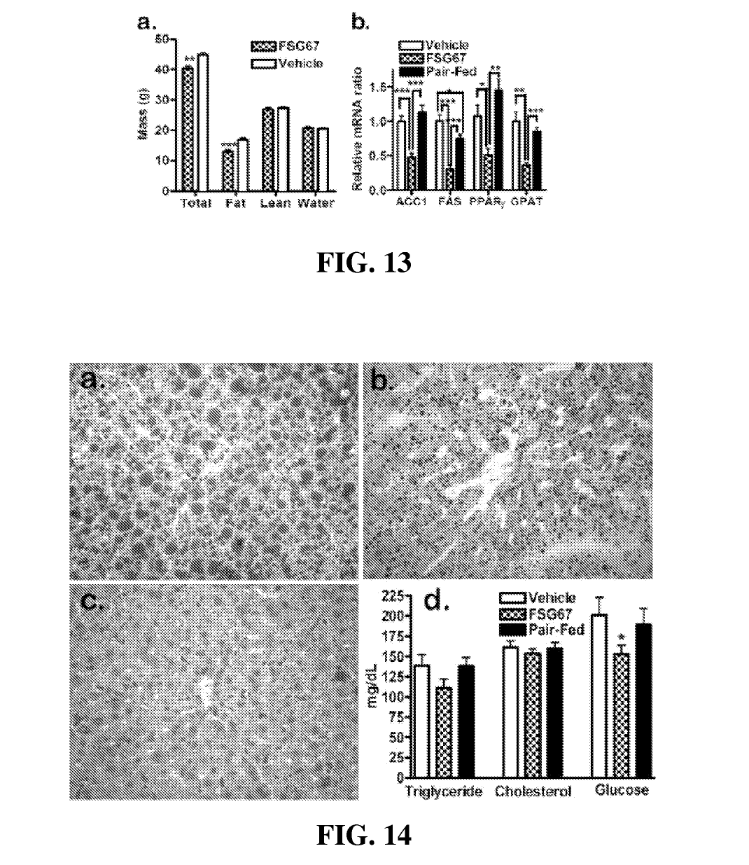

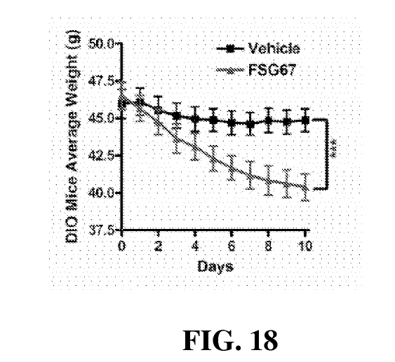

Pharmacological GPAT inhibition reduced adiposity and down-regulated lipogenic gene expression in DIO mice. Since FSG67 increased fatty acid oxidation and reduced food intake in DIO mice, we next used Q-NMR analysis to measure lean, fat and water mass in FSG67 treated and control mice to determine the composition of the tissue loss with FSG67 treatment. In an additional chronic treatment experiment, 10 DIO mice were treated with FSG67 (5 mg/kg/d, ip) and 10 received vehicle for 10 days. The FSG67 treated mice lost 6.1±0.9 g (13.1±1.9%) while vehicle controls lost 1.1±0.4 g (2.3±0.8%) (p<0.0001. 2-way ANOVA). (FIG. 18) Q-NMR analysis demonstrated a 4.0 g reduction in fat mass in the FSG67 treated animals compared to vehicle control (p<0.0001, 2-tailed t-test) but no significant change in lean or water mass (FIG. 13a). At the conclusion of the experiment, the FSG67 treated mice weighed 4.4 g less than the vehicle controls, which could be accounted for by the 4.0 g difference in fat mass. Thus, GPAT inhibition selectively reduces adiposity in DIO mice.

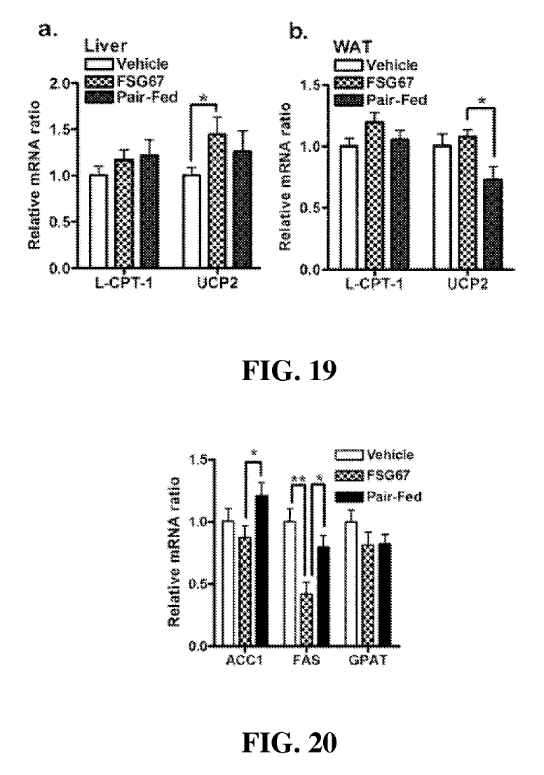

To further explore the mechanism responsible for the reduction in adipose tissue mass, we used real-time RT-PCR to measure the expression of the following key lipogenic genes in white adipose tissue from vehicle control, pair-fed, and FSG67 treated DIO mice from the second indirect calorimetry trial (see FIG. 12c): fatty acid synthase (FAS), responsible for the de novo reductive synthesis of fatty acid 13, acetyl-CoA carboxylase 1 (ACC1), the cytoplasmic isoform of ACC expressed in lipogenic organs that synthesizes malonyl-CoA used as a substrate of FAS for fatty acid synthesis 14, peroxisome proliferator-activated receptor gamma (PPARγ) a key transcription factor for adipogenesis 15, lipid partitioning 16, and postprandial lipid storage 17, and GPAT. After 16 days of treatment, real-time RT-PCR analysis of white adipose tissue from FSG67 treated animals showed substantial down-regulation of ACC1 (p=0.0005 vs. control, p=0.0004 vs. pair-fed), FAS (p=0.0001 vs. control, p=0.0007 vs. pair-fed), PPARy (p=0.032 vs. control, p=0.0019 vs. pair-fed), and GPAT (p=0.0034 vs. control, p=0.0002 vs. pair-fed) (FIG. 13b). Interestingly, uncoupling protein-2 (UCP2) expression was increased in both liver (p=0.043 vs. control) and white adipose tissue (p=0.013, vs. pair-fed) of the FSG67 treated animals which could also contribute to increased fatty acid oxidation 18; L-CPT-1 expression was unaffected. (FIG. 19). Thus, pharmacological GPAT inhibition not only increases fatty acid oxidation and reduces food intake, but up-regulates UCP2 in liver and white adipose tissue while down-regulating lipogenic gene expression in white adipose tissue, all of which should favor a selective decrease in adiposity.

FSG67 substantially reduced serum glucose and triglyceride levels while resolving hepatic steatosis in DIO mice. Consistent with the systemic reduction in adiposity, GPAT inhibition reversed hepatic steatosis in DIO mice. Oil red-O staining of frozen sections of liver showed marked steatosis characterized by large and small droplet triglyceride accumulation in the vehicle treated animals (FIG. 14a). Steatosis was reduced in the pair-fed animals (FIG. 14b) with nearly complete resolution with FSG67 treatment (FIG. 14c). No inflammation, necrosis, or hepatocellular injury was identified. Real-time RT-PCR expression analysis of the hepatic lipogenic genes, ACC1, FAS, and GPAT showed a significant reduction in FAS (p=0.0016 vs. control, p=0.018 vs. pair-fed) and ACC1 (p=0.037 vs. pair-fed) expression but not GPAT, indicating a down-regulation of de novo fatty acid synthesis with FSG67 treatment. (FIG. 20) In addition to the reduction of tissue triglycerides, serum glucose levels were reduced (153.3±10.5 mg/dL) compared with both vehicle control mice (200.6±22.2 mg/dL, p=0.03 2-way ANOVA) and pair-fed (189.0±20.3 mg/dL, p=0.04, 2-way ANOVA). The reduction in serum triglyceride levels seen in the FSG67 treated DIO mice (111.3±10.9 mg/dL) compared to pair-fed (138.5±9.8 mg/dL) or vehicle controls (138.8±13.5 mg/dL) were not statistically significant. Cholesterol levels remained unchanged (FIG. 14d). The resolution of the hepatic steatosis in FSG67 treated mice may have contributed to the normalization of blood glucose levels.

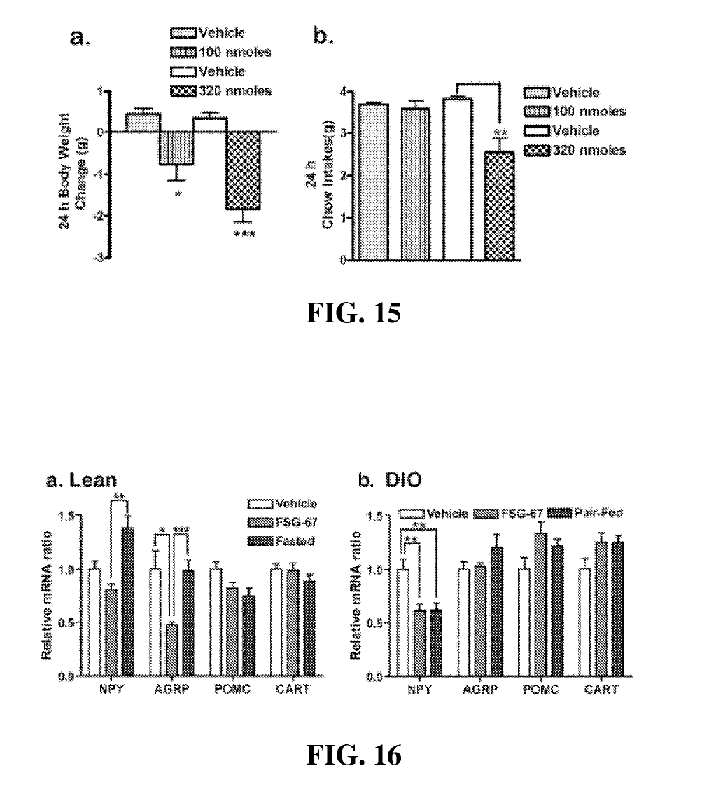

Intracerebroventricular (icy) FSG67 treatment reduced food consumption and body weight. We administered FSG67 icy to determine whether GPAT inhibition acts centrally to reduce food intake. Lean mice were treated with FSG67 icy at doses 100 and 320 nmoles (approximately 300- and 100-fold less than the 5 mg/kg single day systemic dose). Within 24 h, mice treated with 100 nmoles lost 0.75±0.4 g (p=0.016) while the 320 nmole group lost 1.8±0.3 g (p=0.0003); vehicle controls gained 0.43±0.1 g and 0.33±0.1 g respectively (FIG. 15a). The animal weight was regained within 48 h without a significant rebound (data not shown). Significant reduction in chow intake only occurred in the 320 nmole treatment group (3.8±0.1 g control, 2.5±0.3 g FSG67, p=0.0051) (FIG. 15b). Within 48 h, the animals began eating normally with slight hyperphagia in the 320 nmole group on days 3 and 4 (data not shown). These data indicate that the reduction in food consumption accompanying GPAT inhibition may have a significant contribution from the CNS. Moreover, the occurrence of weight loss without a reduction of food intake in the 100 nmole group suggests a central effect on metabolism independent of changes in food intake behaviors.

Acute and chronic FSG67 treatment altered hypothalamic neuropeptide expression. Hypothalamic peptide expression was measured in the lean and DIO mice treated with a single dose of FSG67 (see FIG. 11) and in the chronically treated DIO mice (see FIG. 12) to further asses the mechanism responsible for reduced food intake. In the lean mice treated with a single dose of FSG67, the expression of the orexigenic hypothalamic neuropeptide neuropeptide-Y (NPY) was significantly reduced compared to the fasted animals (p=0.012, 2-tailed t-test) while agouti-related protein (AgRP) expression was substantially diminished compared to both fasted (p=0.020, 2-tailed t-test) and vehicle controls (p=0.0009, 2-tailed t-test) consistent with the acute reduction in food intake (FIG. 16a). Conversely, the anorexigenic neuropeptides, pro-opiomelanocortin (POMC) and cocaine-amphetamine-related transcript (CART) mRNA levels were not affected by food deprivation or acute FSG67 treatment. In contrast to the findings in lean mice, single dose FSG67 treatment of DIO mice significantly increased AgRP expression over that in the vehicle controls and food-deprived animals (data not shown). Notably, food deprivation did not result in increased levels of hypothalamic NPY or AgRP message in DIO mice as was seen with the lean animals. This pattern of increased orexigenic neuropeptide expression with treatment is consistent with a hunger response and may indicate a rebound of orexigenic peptide expression in the DIO mice or could represent a further example of dysregulated neuropeptide signaling in DIO mice 19. In the chronically treated DIO mice, however, hypothalamic neuropeptide analysis showed a significant reduction in NPY expression in both FSG67 (p=0.0052, 2-tailed t-test) and pair-fed animals (p=0.0074, 2-tailed t-test) compared to vehicle controls (FIG. 16b). This profile was more similar to the acutely treated lean mice, and may reflect normalization of the appetite response in the chronically treated DIO mice.

1.-58. (canceled)

59. A compound comprising a formula I: 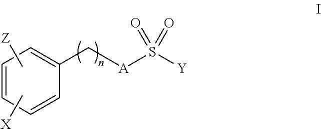
wherein
n is either 0 or 1;
A is selected from the group consisting of NR¹, O, and S, wherein le is selected from the group consisting of H, hydroxyl, C₁-C₁₀ alkyl, C₁-C₁₀ alkoxy, alkenyl, aryl, alkylaryl and arylalkyl;
X is selected from the group consisting of a carboxylate residue, a phosphonate residue, a phosphate residue, and a C₁-C₁₀ alkyl residue which is optionally substituted with one or more residues selected from the group consisting of a carboxylate residue, a phosphonate residue and a phosphate residue;
Y is selected from the group consisting of C₁-C₂₀ alkyl, alkenyl, halide, hydroxyl, C₁-C₂₀ alkoxy, aryl, alkylaryl, arylalkyl, cycloalkyl, cycloalkenyl and a heterocyclic ring, any of which are optionally substituted at one or more positions with a halogen; and
Z is selected from the group consisting of H, a hydroxyl group, a halide, an aryl group, an alkylaryl group, an arylalkyl group, a cycloalkyl group, a cycloalkenyl group and a heterocyclic ring, any of which are is optionally substituted at one or more positions with one or a combination of substitution groups selected from the group consisting of a C₁-₁₀ alkyl group, C₁-₁₀ alkoxy group, a hydroxyl group, a cyano group, a carboxylate group, a halide, an aryl group, an alkylaryl group, an arylalkyl group, a cycloalkyl group, a cycloalkenyl group and a heterocyclic ring.

60. The compound of claim 59, wherein A is NR¹ wherein R¹ is a hydrogen.

61. The compound of claim 59, wherein X is a carboxylic acid residue.

62. The compound of claim 59, wherein X is a phosphonate residue.

63. The compound of claim 59, wherein X is in the ortho or meta position with respect to the sulfonyl linker of the phenyl ring.

64. The compound of claim 59, wherein Y is a C₁-C₂₀ alkyl group selected from the group consisting of C₅H₁₁, C₈H₁₇, C₉H₁₉, and C₁₄H₂₉.

65. The compound of claim 59, wherein Y is 4-ClPh.

66. The compound of claim 59, wherein Z is hydrogen or an optionally substituted aryl group.

67. The compound of claim 59 selected from the group consisting of

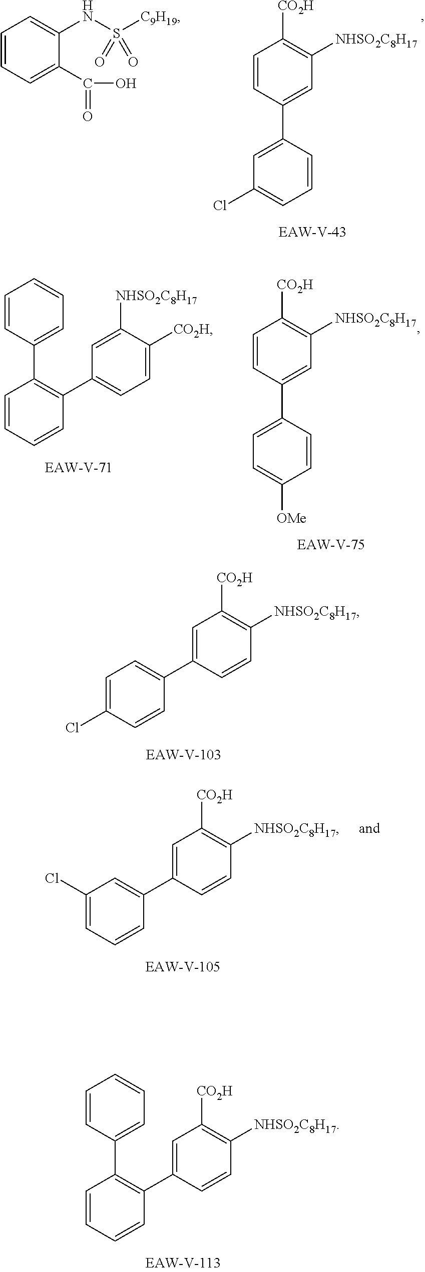

68. A compound comprising a formula IVa: 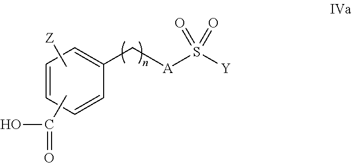
wherein
n is 0;
A is selected from the group consisting of NR¹, O, and S, wherein R¹ is selected from the group consisting of H, hydroxyl, C₁-C₁₀ alkyl, C₁-C₁₀ alkoxy, alkenyl, aryl, alkylaryl and arylalkyl;
Y is selected from the group consisting of C₁-C₂₀ alkyl, alkenyl, halide, hydroxyl, C₁-C₂₀ alkoxy, aryl, alkylaryl, arylalkyl, cycloalkyl, cycloalkenyl and a heterocyclic ring, any of which are optionally substituted at one or more positions with a halogen; and
Z is selected from the group consisting of H, a hydroxyl group, a halide, an aryl group, an alkylaryl group, an arylalkyl group, a cycloalkyl group, a cycloalkenyl group and a heterocyclic ring, any of which are optionally substituted at one or more positions with one or a combination of substitution groups selected from the group consisting of a C₁-₁₀ alkyl group, C₁-₁₀ alkoxy group, a hydroxyl group, a cyano group, a carboxylate group, a halide, an aryl group, an alkylaryl group, an arylalkyl group, a cycloalkyl group, a cycloalkenyl group and a heterocyclic ring.

69. The compound of claim 68, wherein A is comprised of NR¹ wherein R¹ is a hydrogen.

70. The compound of claim 69, wherein COOH is in an ortho or meta position with respect to the sulfonyl linker of the phenyl ring.

71. The compound of claim 68, wherein Y is a C₁-C₂₀ alkyl group selected from the group consisting of C₅H₁₁, C₈H₁₇, C₉H₁₉, and C₁₄H₂₉.

72. The compound of claim 68, wherein Y is 4-ClPh.

73. The compound of claim 68, wherein Z is hydrogen or an optionally substituted aryl group.

74. A pharmaceutical composition comprising a pharmaceutical diluent and a compound according to claim 59.

75. The pharmaceutical composition of claim 74, wherein the compound is selected from the group consisting of

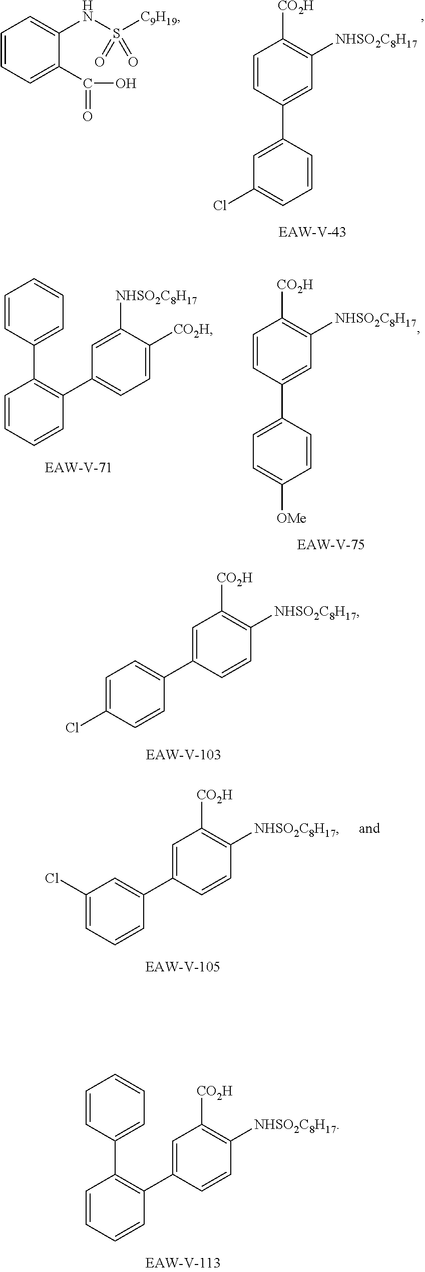

76. A method of inducing weight loss in a subject comprising administering an effective amount of a pharmaceutical composition according to claim 74 to the subject.

77. The method of inducing weight loss in a subject according to claim 76, wherein the compound is selected from the group consisting of

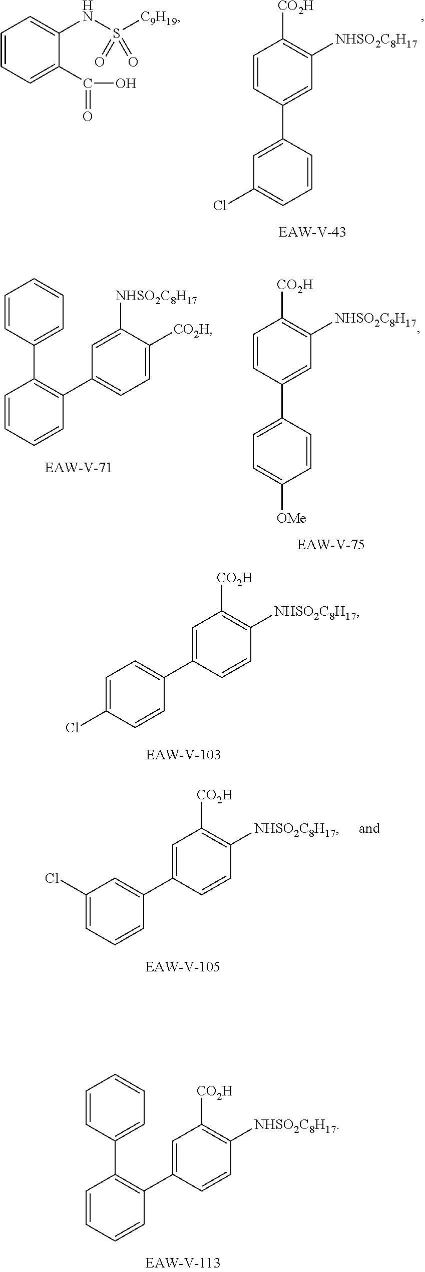

78. A method of inhibiting glycerol 3-phosphate acyltransferase activity within a subject comprising administering an effective amount of a pharmaceutical composition according to claim 74 to the subject.

79. The method of inhibiting glycerol 3-phosphate acyltransferase activity according to claim 78, wherein the compound is selected from the group consisting of

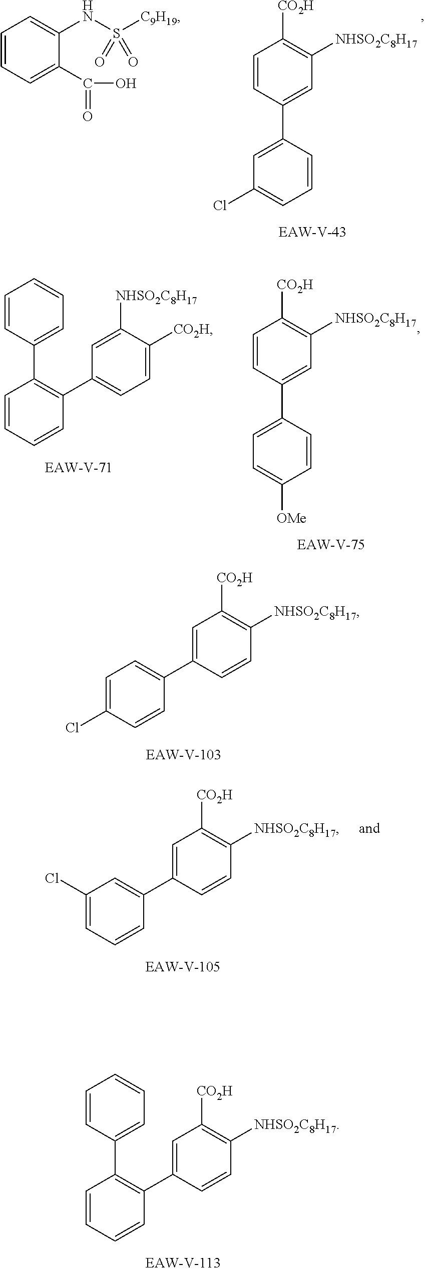

80. A method of increasing fatty acid oxidation in a subject comprising administering an effective amount of a pharmaceutical composition according to claim 74 to the subject.

81. The method of increasing fatty acid oxidation according to claim 80, wherein the compound is selected from the group consisting of [chemical structure]
